SEQUENCE LISTING (193 sequences)

SEQ ID NO: 1 - DNA, 357 bp, Artificial Sequence
  DNA sequence of Cpd.1
atgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaa

SEQ ID NO: 2 - RNA, 357 nt, Artificial Sequence
  RNA sequence of Cpd.1
  modified_base (1)..(357): m1f (uridine is 1-methylpseudouridine)
augggcaagauuagcagccugccuacacagcuguucaagugcugcuucugcgacuuccugaaagugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcuguuucagaagcugcgaccugcggcggcuggaaauguauugugccccucugaagccugccaagagcgccuaa

SEQ ID NO: 3 - DNA, 366 bp, Artificial Sequence
  DNA sequence of Cpd.2
atgggaatacctatgggcaagagcatgctggtgctgctgaccttcctggccttcgcctcttgttgtatcgccgtgaagatgcacaccatgagcagcagccacctgttctatctggccctgtgcctgctgacattcaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 4 - RNA, 366 nt, Artificial Sequence
  RNA sequence of Cpd.2
  modified_base (1)..(366): m1f (uridine is 1-methylpseudouridine)
augggaauaccuaugggcaagagcaugcuggugcugcugaccuuccuggccuucgccucuuguuguaucgccgugaagaugcacaccaugagcagcagccaccuguucuaucuggcccugugccugcugacauucaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 5 - DNA, 348 bp, Artificial Sequence
  DNA sequence of Cpd.3
atgaaatgggtcacctttatcagcctgctgttcctgttcagcagcgcctacagcgtgaagatgcacaccatgagcagcagccacctgttctatctggccctgtgcctgctgaccttcacaagctctgctaccgccggacctgagacactgtgtggtgctgaactggtggacgccctgcagttcgtgtgtggcgatagaggcttctacttcaacaagcccaccggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 6 - RNA, 348 nt, Artificial Sequence
  RNA sequence of Cpd.3
  modified_base (1)..(348): m1f (uridine is 1-methylpseudouridine)
augaaaugggucaccuuuaucagccugcuguuccuguucagcagcgccuacagcgugaagaugcacaccaugagcagcagccaccuguucuaucuggcccugugccugcugaccuucacaagcucugcuaccgccggaccugagacacuguguggugcugaacugguggacgcccugcaguucguguguggcgauagaggcuucuacuucaacaagcccaccggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 7 - DNA, 348 bp, Artificial Sequence
  DNA sequence of Cpd.4
atgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 8 - RNA, 348 nt, Artificial Sequence
  RNA sequence of Cpd.4
  modified_base (1)..(348): m1f (uridine is 1-methylpseudouridine)
augaccauccuguuucugacaauggucaucagcuacuucggcugcaugaaggccgugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 9 - DNA, 357 bp, Artificial Sequence
  DNA sequence of Cpd.5
atgaatgccaaagtggtggtggtgctggtcctggtgctgacagctctgtgtctgtccgacggcgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 10 - RNA, 357 nt, Artificial Sequence
  RNA sequence of Cpd.5
  modified_base (1)..(357): m1f (uridine is 1-methylpseudouridine)
augaaugccaaaguggugguggugcugguccuggugcugacagcucugugucuguccgacggcgugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 11 - DNA, 351 bp, Artificial Sequence
  DNA sequence of Cpd.6
atgggctggtcctgcatcatcctgtttctggtggccacagccacaggcgtgcacagcgtgaagatgcacacaatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 12 - RNA, 351 nt, Artificial Sequence
  RNA sequence of Cpd.6
  modified_base (1)..(351): m1f (uridine is 1-methylpseudouridine)
augggcugguccugcaucauccuguuucugguggccacagccacaggcgugcacagcgugaagaugcacacaaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 13 - DNA, 351 bp, Artificial Sequence
  DNA sequence of Cpd.7
atggactggacttggagaatcctgttcctggtggccgctgccacaggcgcccattctgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 14 - RNA, 351 nt, Artificial Sequence
  RNA sequence of Cpd.7
  modified_base (1)..(351): m1f (uridine is 1-methylpseudouridine)
auggacuggacuuggagaauccuguuccugguggccgcugccacaggcgcccauucugugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 15 - DNA, 3450 bp, Artificial Sequence
  DNA sequence of vector pVAX.A120 with Cpd.1
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaagaattctgcagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 16 - DNA, 2758 bp, Artificial Sequence
  DNA sequence of vector pMA-T with Cpd.2
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcggatccgccaccatgggaatacctatgggcaagagcatgctggtgctgctgaccttcctggccttcgcctcttgttgtatcgccgtgaagatgcacaccatgagcagcagccacctgttctatctggccctgtgcctgctgacattcaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaagaattcggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 17 - DNA, 2740 bp, Artificial Sequence
  DNA sequence of vector pMA-T with Cpd.3
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcggatccgccaccatgaaatgggtcacctttatcagcctgctgttcctgttcagcagcgcctacagcgtgaagatgcacaccatgagcagcagccacctgttctatctggccctgtgcctgctgaccttcacaagctctgctaccgccggacctgagacactgtgtggtgctgaactggtggacgccctgcagttcgtgtgtggcgatagaggcttctacttcaacaagcccaccggctacggcagcagctctagaagggctcctcagaccggaatcgtggacgagtgctgcttcagaagctgcgacctgcggcggctggaaatgtattgtgcccctctgaagcctgccaagagcgcctaagaattcggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 18 - DNA, 2740 bp, Artificial Sequence
  DNA sequence of vector pMA-T with Cpd.4
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcggatccgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaagaattcggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 19 - DNA, 2749 bp, Artificial Sequence
  DNA sequence of vector pMA-T with Cpd.5
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcggatccgccaccatgaatgccaaagtggtggtggtgctggtcctggtgctgacagctctgtgtctgtccgacggcgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaagaattcggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 20 - DNA, 2710 bp, Artificial Sequence
  DNA sequence of vector pMA-RQ with Cpd.6
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggatccgccaccatgggctggtcctgcatcatcctgtttctggtggccacagccacaggcgtgcacagcgtgaagatgcacacaatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaagaattcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 21 - DNA, 2710 bp, Artificial Sequence
  DNA sequence of vector pMA-RQ with Cpd.7
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggatccgccaccatggactggacttggagaatcctgttcctggtggccgctgccacaggcgcccattctgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaagaattcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 22 - DNA, 20 bp, Artificial Sequence
  Forward Primer
gctgcaaggcgattaagttg

SEQ ID NO: 23 - DNA, 140 bp, Artificial Sequence
  Reverse Primer
  modified_base (1)..(3): um
tttttttttttttttttttttttttttttttttttttttttttttttttttttttttttttttttttttttttttttttttttttttttttttttttttttttttttttttttttttcagctatgaccatgttaatgcag

SEQ ID NO: 24 - DNA, 63 bp, Artificial Sequence
  IGF1 signal peptide
atgggcaagattagcagcctgcctacacagctgttcaagtgctgcttctgcgacttcctgaaa

SEQ ID NO: 25 - protein, 21 aa, Homo sapiens
MGKISSLPTQLFKCCFCDFLK

SEQ ID NO: 26 - DNA, 72 bp, Artificial Sequence
  IGF2 signal peptide
atgggaatacctatgggcaagagcatgctggtgctgctgaccttcctggccttcgcctcttgttgtatcgcc

SEQ ID NO: 27 - protein, 24 aa, Homo sapiens
MGIPMGKSMLVLLTFLAFASCCIA

SEQ ID NO: 28 - DNA, 54 bp, Artificial Sequence
  ALB signal peptide
atgaaatgggtcacctttatcagcctgctgttcctgttcagcagcgcctacagc

SEQ ID NO: 29 - protein, 18 aa, Homo sapiens
MKWVTFISLLFLFSSAYS

SEQ ID NO: 30 - DNA, 54 bp, Artificial Sequence
  BDNF signal peptide
atgaccatcctgtttctgacaatggtcatcagctacttcggctgcatgaaggcc

SEQ ID NO: 31 - protein, 18 aa, Homo sapiens
MTILFLTMVISYFGCMKA

SEQ ID NO: 32 - DNA, 63 bp, Artificial Sequence
  CXCL12 signal peptide
atgaatgccaaagtggtggtggtgctggtcctggtgctgacagctctgtgtctgtccgacggc

SEQ ID NO: 33 - protein, 21 aa, Homo sapiens
MNAKVVVVLVLVLTALCLSDG

SEQ ID NO: 34 - DNA, 57 bp, Artificial Sequence
  Synthetic SEQ1 signal peptide
atgggctggtcctgcatcatcctgtttctggtggccacagccacaggcgtgcacagc

SEQ ID NO: 35 - protein, 19 aa, Artificial Sequence
  Synthetic SEQ1 signal peptide
MGWSCIILFLVATATGVHS

SEQ ID NO: 36 - DNA, 57 bp, Artificial Sequence
  Synthetic SEQ2 signal peptide
atggactggacttggagaatcctgttcctggtggccgctgccacaggcgcccattct

SEQ ID NO: 37 - protein, 19 aa, Artificial Sequence
  Synthetic SEQ2 signal peptide
MDWTWRILFLVAAATGAHS

SEQ ID NO: 38 - DNA, 81 bp, Artificial Sequence
  Propeptide of human IGF1
gtgaagatgcacaccatgagcagcagccacctgttctatctggccctgtgcctgctgacctttaccagctctgctaccgcc

SEQ ID NO: 39 - DNA, 210 bp, Artificial Sequence
  Mature human IGF1
ggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcc

SEQ ID NO: 40 - DNA, 2749 bp, Artificial Sequence
  DNA sequcence of vector_pMA-T with compound 1
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcggatccgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaagaattcggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 41 - protein, 35 aa, Homo sapiens
MRPRTKARSPGRALRNPWRGFLPLTLALFVGAGHA

SEQ ID NO: 42 - DNA, 105 bp, Artificial Sequence
  LTBP2 SP
atgcggcccaggaccaaagccagatctcctggcagagccctgcggaatccttggagaggattcctgcctctgacactggccctgtttgttggagccggacacgcc

SEQ ID NO: 43 - DNA, 399 bp, Artificial Sequence
  DNA sequence of Cpd.08
atgcggcccaggaccaaagccagatctcctggcagagccctgcggaatccttggagaggattcctgcctctgacactggccctgtttgttggagccggacacgccgtgaagatgcacacaatgagcagcagccacctgttctacctggctctgtgcctgctgacctttaccagctctgccacagccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 44 - RNA, 399 nt, Artificial Sequence
  RNA sequence of Cpd.08
  modified_base (1)..(399): m1f (uridine is 1-methylpseudouridine)
augcggcccaggaccaaagccagaucuccuggcagagcccugcggaauccuuggagaggauuccugccucugacacuggcccuguuuguuggagccggacacgccgugaagaugcacacaaugagcagcagccaccuguucuaccuggcucugugccugcugaccuuuaccagcucugccacagccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 45 - DNA, 2859 bp, Artificial Sequence
  DNA sequence of vector_ pMA-RQ with Cpd.08
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggatcctatcatccctttgaatggaagcctccgtaaaacgacggccagttaatacgactcactatagggccaccatgcggcccaggaccaaagccagatctcctggcagagccctgcggaatccttggagaggattcctgcctctgacactggccctgtttgttggagccggacacgccgtgaagatgcacacaatgagcagcagccacctgttctacctggctctgtgcctgctgacctttaccagctctgccacagccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaacaggaaacagctatgacgcaaagcgttgagagtggtaacgaattcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 46 - protein, 27 aa, Homo sapiens
MALRKGGLALALLLLSWVALGPRSLEG

SEQ ID NO: 47 - DNA, 81 bp, Artificial Sequence
  IGFALS SP
atggctctgcggaaaggtggacttgctctggctctgctgctgctgtcttgggttgcactgggccctagatctctggaaggc

SEQ ID NO: 48 - DNA, 375 bp, Artificial Sequence
  DNA sequence of Cpd.09
atggctctgcggaaaggtggacttgctctggctctgctgctgctgtcttgggttgcactgggccctagatctctggaaggcgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 49 - RNA, 375 nt, Artificial Sequence
  RNA sequence of Cpd.09
  modified_base (1)..(375): m1f (uridine is 1-methylpseudouridine)
auggcucugcggaaagguggacuugcucuggcucugcugcugcugucuuggguugcacugggcccuagaucucuggaaggcgugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 50 - DNA, 2835 bp, Artificial Sequence
  NA sequence of vector_ pMA-RQ with Cpd.9
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggatcctatcatccctttgaatggaagcctccgtaaaacgacggccagttaatacgactcactatagggccaccatggctctgcggaaaggtggacttgctctggctctgctgctgctgtcttgggttgcactgggccctagatctctggaaggcgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaacaggaaacagctatgacgcaaagcgttgagagtggtaacgaattcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 51 - protein, 24 aa, Homo sapiens
MALWMRLLPLLALLALWGPDPAAA

SEQ ID NO: 52 - DNA, 72 bp, Artificial Sequence
  INS SP
atggctctgtggatgagactgctgcctctgctggcactgcttgctctgtggggacctgatcctgccgccgct

SEQ ID NO: 53 - DNA, 366 bp, Artificial Sequence
  DNA sequence of Cpd.10
atggctctgtggatgagactgctgcctctgctggcactgcttgctctgtggggacctgatcctgccgccgctgtgaagatgcacacaatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 54 - RNA, 366 nt, Artificial Sequence
  RNA sequence of Cpd.10
  modified_base (1)..(366): m1f (uridine is 1-methylpseudouridine)
auggcucuguggaugagacugcugccucugcuggcacugcuugcucuguggggaccugauccugccgccgcugugaagaugcacacaaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 55 - DNA, 2713 bp, Artificial Sequence
  DNA sequence of vector_ pMA-RQ with Cpd.10
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatggctctgtggatgagactgctgcctctgctggcactgcttgctctgtggggacctgatcctgccgccgctgtgaagatgcacacaatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 56 - protein, 27 aa, Homo sapiens
MGVHECPAWLWLLLSLLSLPLGLPVLG

SEQ ID NO: 57 - DNA, 81 bp, Artificial Sequence
  Epo SP
atgggagtgcatgaatgtcctgcttggctgtggctgctgctgagcctgctgtctctgcctctgggactgcctgtgctggga

SEQ ID NO: 58 - DNA, 375 bp, Artificial Sequence
  DNA sequence of Cpd.11
atgggagtgcatgaatgtcctgcttggctgtggctgctgctgagcctgctgtctctgcctctgggactgcctgtgctgggagtgaagatgcacaccatgagcagctcccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 59 - RNA, 375 nt, Artificial Sequence
  RNA sequence of Cpd.11
  modified_base (1)..(375): m1f (uridine is 1-methylpseudouridine)
augggagugcaugaauguccugcuuggcuguggcugcugcugagccugcugucucugccucugggacugccugugcugggagugaagaugcacaccaugagcagcucccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 60 - DNA, 2722 bp, Artificial Sequence
  DNA sequence of vector_ pMA-RQ with Cpd.11
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggagtgcatgaatgtcctgcttggctgtggctgctgctgagcctgctgtctctgcctctgggactgcctgtgctgggagtgaagatgcacaccatgagcagctcccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 61 - protein, 29 aa, Homo sapiens
MAGPATQSPMKLMALQLLLWHSALWTVQE

SEQ ID NO: 62 - DNA, 87 bp, Artificial Sequence
  CSF3 SP
atggccggacctgccacacagtctccaatgaagctgatggccctgcagctgctgctgtggcattctgctctgtggaccgtgcaagaa

SEQ ID NO: 63 - DNA, 381 bp, Artificial Sequence
  DNA sequence of Cpd.12
atggccggacctgccacacagtctccaatgaagctgatggccctgcagctgctgctgtggcattctgctctgtggaccgtgcaagaagtgaagatgcacaccatgagcagcagccacctgttctatctggccctgtgcctgctgacctttaccagctctgctaccgccggaccagagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 64 - RNA, 381 nt, Artificial Sequence
  RNA sequence of Cpd.12
  modified_base (1)..(381): m1f (uridine is 1-methylpseudouridine)
auggccggaccugccacacagucuccaaugaagcugauggcccugcagcugcugcuguggcauucugcucuguggaccgugcaagaagugaagaugcacaccaugagcagcagccaccuguucuaucuggcccugugccugcugaccuuuaccagcucugcuaccgccggaccagagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 65 - DNA, 2841 bp, Artificial Sequence
  DNA sequence of vector_ pMA-RQ with Cpd.12
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggatcctatcatccctttgaatggaagcctccgtaaaacgacggccagttaatacgactcactatagggccaccatggccggacctgccacacagtctccaatgaagctgatggccctgcagctgctgctgtggcattctgctctgtggaccgtgcaagaagtgaagatgcacaccatgagcagcagccacctgttctatctggccctgtgcctgctgacctttaccagctctgctaccgccggaccagagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaacaggaaacagctatgacgcaaagcgttgagagtggtaacgaattcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 66 - protein, 18 aa, Homo sapiens
MSMLFYTLITAFLIGIQA

SEQ ID NO: 67 - DNA, 54 bp, Artificial Sequence
  NGF SP
atgagcatgctgttctataccctgatcaccgcctttctgatcggcatccaggcc

SEQ ID NO: 68 - DNA, 348 bp, Artificial Sequence
  DNA sequence of Cpd.13
atgagcatgctgttctataccctgatcaccgcctttctgatcggcatccaggccgtgaagatgcacaccatgtctagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 69 - RNA, 348 nt, Artificial Sequence
  RNA sequence of Cpd.13
  modified_base (1)..(348): m1f (uridine is 1-methylpseudouridine)
augagcaugcuguucuauacccugaucaccgccuuucugaucggcauccaggccgugaagaugcacaccaugucuagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 70 - DNA, 2808 bp, Artificial Sequence
  DNA sequence of vector_ pMA-RQ with Cpd.13
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggatcctatcatccctttgaatggaagcctccgtaaaacgacggccagttaatacgactcactatagggccaccatgagcatgctgttctataccctgatcaccgcctttctgatcggcatccaggccgtgaagatgcacaccatgtctagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaacaggaaacagctatgacgcaaagcgttgagagtggtaacgaattcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 71 - DNA, 333 bp, Artificial Sequence
  DNA sequence of Cpd.30
atggctctgtggatgagactgctgcctctgctggcactgcttgctctgtggggacctgatcctgccgccgcttttgtgaatcagcacctgtgtggcagccacctggtggaagccctgtatctcgtgtgtggcgagcggggcttcttctacacccctaagacaagaagagaggccgaggacctgcaagtcggccaggttgaacttggcggaggacctggtgctggatctctgcaacctctggctctggaaggcagcctgcagaagaggggcatcgtcgagcagtgctgcaccagcatctgtagcctgtaccagctggaaaactactgcaactga

SEQ ID NO: 72 - RNA, 333 nt, Artificial Sequence
  RNA sequence of Cpd.30
  modified_base (1)..(333): m1f (uridine is 1-methylpseudouridine)
auggcucuguggaugagacugcugccucugcuggcacugcuugcucuguggggaccugauccugccgccgcuuuugugaaucagcaccuguguggcagccaccugguggaagcccuguaucucguguguggcgagcggggcuucuucuacaccccuaagacaagaagagaggccgaggaccugcaagucggccagguugaacuuggcggaggaccuggugcuggaucucugcaaccucuggcucuggaaggcagccugcagaagaggggcaucgucgagcagugcugcaccagcaucuguagccuguaccagcuggaaaacuacugcaacuga

SEQ ID NO: 73 - DNA, 2680 bp, Artificial Sequence
  DNA sequence of vector_ pMA-RQ with Cpd.30
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatggctctgtggatgagactgctgcctctgctggcactgcttgctctgtggggacctgatcctgccgccgcttttgtgaatcagcacctgtgtggcagccacctggtggaagccctgtatctcgtgtgtggcgagcggggcttcttctacacccctaagacaagaagagaggccgaggacctgcaagtcggccaggttgaacttggcggaggacctggtgctggatctctgcaacctctggctctggaaggcagcctgcagaagaggggcatcgtcgagcagtgctgcaccagcatctgtagcctgtaccagctggaaaactactgcaactgactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 74 - DNA, 582 bp, Artificial Sequence
  DNA sequence of Cpd.27
atgggagtgcatgaatgtcctgcttggctgtggctgctgctgagcctgctgtctctgcctctgggactgcctgttcttggagcccctcctagactgatctgcgacagcagagtgctggaaagatacctgctggaagccaaagaggccgagaacatcaccacaggctgtgccgagcactgcagcctgaacgagaatatcaccgtgcctgacaccaaagtgaacttctacgcctggaagcggatggaagtgggccagcaggctgtggaagtttggcaaggactggccctgctgagcgaagctgttctgagaggacaggctctgctggtcaacagctctcagccttgggaacctctgcaactgcacgtggacaaggccgtgtctggcctgagaagcctgaccacactgctgagagcactgggagcccagaaagaggccatctctccacctgatgctgcctctgctgcccctctgagaaccatcaccgccgacaccttcagaaagctgttccgggtgtacagcaacttcctgcggggcaagctgaagctgtacacaggcgaggcttgcagaaccggcgacagataa

SEQ ID NO: 75 - RNA, 582 nt, Artificial Sequence
  RNA sequence of Cpd.27
  modified_base (1)..(582): m1f (uridine is 1-methylpseudouridine)
augggagugcaugaauguccugcuuggcuguggcugcugcugagccugcugucucugccucugggacugccuguucuuggagccccuccuagacugaucugcgacagcagagugcuggaaagauaccugcuggaagccaaagaggccgagaacaucaccacaggcugugccgagcacugcagccugaacgagaauaucaccgugccugacaccaaagugaacuucuacgccuggaagcggauggaagugggccagcaggcuguggaaguuuggcaaggacuggcccugcugagcgaagcuguucugagaggacaggcucugcuggucaacagcucucagccuugggaaccucugcaacugcacguggacaaggccgugucuggccugagaagccugaccacacugcugagagcacugggagcccagaaagaggccaucucuccaccugaugcugccucugcugccccucugagaaccaucaccgccgacaccuucagaaagcuguuccggguguacagcaacuuccugcggggcaagcugaagcuguacacaggcgaggcuugcagaaccggcgacagauaa

SEQ ID NO: 76 - DNA, 2929 bp, Artificial Sequence
  DNA sequence of vector_ pMA-RQ with Cpd.27
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggagtgcatgaatgtcctgcttggctgtggctgctgctgagcctgctgtctctgcctctgggactgcctgttcttggagcccctcctagactgatctgcgacagcagagtgctggaaagatacctgctggaagccaaagaggccgagaacatcaccacaggctgtgccgagcactgcagcctgaacgagaatatcaccgtgcctgacaccaaagtgaacttctacgcctggaagcggatggaagtgggccagcaggctgtggaagtttggcaaggactggccctgctgagcgaagctgttctgagaggacaggctctgctggtcaacagctctcagccttgggaacctctgcaactgcacgtggacaaggccgtgtctggcctgagaagcctgaccacactgctgagagcactgggagcccagaaagaggccatctctccacctgatgctgcctctgctgcccctctgagaaccatcaccgccgacaccttcagaaagctgttccgggtgtacagcaacttcctgcggggcaagctgaagctgtacacaggcgaggcttgcagaaccggcgacagataactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 77 - protein, 24 aa, Homo sapiens
MGLTSQLLPPLFFLLACAGNFVHG

SEQ ID NO: 78 - DNA, 72 bp, Artificial Sequence
  IL4 SP
atgggactgacatctcaactgctgcctccactgttctttctgctggcctgcgccggcaattttgtgcacggc

SEQ ID NO: 79 - DNA, 462 bp, Artificial Sequence
  DNA sequence of Cpd.33
atgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctga

SEQ ID NO: 80 - RNA, 462 nt, Artificial Sequence
  RNA sequence of Cpd.33
  modified_base (1)..(462): m1f (uridine is 1-methylpseudouridine)
augggacugacaucucaacugcugccuccacuguucuuucugcuggccugcgccggcaauuuugugcacggccacaagugcgacaucacccugcaagagaucaucaagacccugaacagccugaccgagcagaaaacccugugcaccgagcugaccgugaccgauaucuuugccgccagcaagaacacaaccgagaaagagacauucugcagagccgccaccgugcugagacaguucuacagccaccacgagaaggacaccagaugccugggagcuacagcccagcaguuccacagacacaagcagcugauccgguuccugaagcggcuggacagaaaucuguggggacucgccggccugaauagcugcccugugaaagaggccaaccagucuacccuggaaaacuuccuggaacggcugaaaaccaucaugcgcgagaaguacagcaagugcagcagcuga

SEQ ID NO: 81 - DNA, 2809 bp, Artificial Sequence
  DNA sequence of vector_ pMA-RQ with Cpd.33
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 82 - protein, 18 aa, Homo sapiens
MHSSALLCCLVLLTGVRA

SEQ ID NO: 83 - DNA, 54 bp, Artificial Sequence
  IL10 SP
atgcatagctctgccctgctgtgttgtctggtgctgctgacaggcgtcagagcc

SEQ ID NO: 84 - DNA, 537 bp, Artificial Sequence
  DNA sequence of Cpd.36
atgcatagctctgccctgctgtgttgtctggtgctgctgacaggcgtcagagcctctcctggacagggaacccagagcgagaatagctgcacccactttccaggcaacctgcctaacatgctgcgggacctgagagatgccttcagcagagtgaaaacattcttccagatgaaggaccagctggacaacctgctgctgaaagagagcctgctggaagatttcaagggctacctgggctgtcaggccctgagcgagatgatccagttctacctggaagaagtgatgccccaggccgagaatcaggaccccgatattaaggcccacgtgaacagcctgggcgagaacctgaaaaccctgcggctgagactgcggcggtgccacagatttctgccttgcgagaacaagagcaaggccgtggaacaagtgaagaacgccttcaacaagctgcaagagaagggcatctacaaggccatgtccgagttcgacatcttcatcaactacatcgaggcctacatgaccatgaagatccggaactga

SEQ ID NO: 85 - RNA, 537 nt, Artificial Sequence
  RNA sequence of Cpd.36
  modified_base (1)..(537): m1f (uridine is 1-methylpseudouridine)
augcauagcucugcccugcuguguugucuggugcugcugacaggcgucagagccucuccuggacagggaacccagagcgagaauagcugcacccacuuuccaggcaaccugccuaacaugcugcgggaccugagagaugccuucagcagagugaaaacauucuuccagaugaaggaccagcuggacaaccugcugcugaaagagagccugcuggaagauuucaagggcuaccugggcugucaggcccugagcgagaugauccaguucuaccuggaagaagugaugccccaggccgagaaucaggaccccgauauuaaggcccacgugaacagccugggcgagaaccugaaaacccugcggcugagacugcggcggugccacagauuucugccuugcgagaacaagagcaaggccguggaacaagugaagaacgccuucaacaagcugcaagagaagggcaucuacaaggccauguccgaguucgacaucuucaucaacuacaucgaggccuacaugaccaugaagauccggaacuga

SEQ ID NO: 86 - DNA, 2884 bp, Artificial Sequence
  DNA sequence of vector_ pMA-RQ with Cpd.36
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgcatagctctgccctgctgtgttgtctggtgctgctgacaggcgtcagagcctctcctggacagggaacccagagcgagaatagctgcacccactttccaggcaacctgcctaacatgctgcgggacctgagagatgccttcagcagagtgaaaacattcttccagatgaaggaccagctggacaacctgctgctgaaagagagcctgctggaagatttcaagggctacctgggctgtcaggccctgagcgagatgatccagttctacctggaagaagtgatgccccaggccgagaatcaggaccccgatattaaggcccacgtgaacagcctgggcgagaacctgaaaaccctgcggctgagactgcggcggtgccacagatttctgccttgcgagaacaagagcaaggccgtggaacaagtgaagaacgccttcaacaagctgcaagagaagggcatctacaaggccatgtccgagttcgacatcttcatcaactacatcgaggcctacatgaccatgaagatccggaactgactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 87 - protein, 20 aa, Homo sapiens
MSLSFLLLLFFSHLILSAWA

SEQ ID NO: 88 - DNA, 60 bp, Artificial Sequence
  FGF5 SP
atgagcctgagctttctgctgctgctgttcttcagccacctgatcctgtctgcctgggcc

SEQ ID NO: 89 - DNA, 354 bp, Artificial Sequence
  DNA sequence of Cpd.14
atgagcctgagctttctgctgctgctgttcttcagccacctgatcctgtctgcctgggccgtgaagatgcacaccatgtccagctctcacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 90 - RNA, 354 nt, Artificial Sequence
  RNA sequence of Cpd.14
  modified_base (1)..(354): m1f (uridine is 1-methylpseudouridine)
augagccugagcuuucugcugcugcuguucuucagccaccugauccugucugccugggccgugaagaugcacaccauguccagcucucaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 91 - DNA, 2701 bp, Artificial Sequence
  DNA sequence of vector_ pMA-RQ with Cpd.14
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgagcctgagctttctgctgctgctgttcttcagccacctgatcctgtctgcctgggccgtgaagatgcacaccatgtccagctctcacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 92 - protein, 18 aa, Homo sapiens
MWLLVSVILISRISSVGG

SEQ ID NO: 93 - DNA, 54 bp, Artificial Sequence
  FHR2 SP
atgtggctgctggtgtctgtgatcctgatcagccggatctcttctgtcggcggc

SEQ ID NO: 94 - DNA, 348 bp, Artificial Sequence
  DNA sequence of Cpd.15
atgtggctgctggtgtctgtgatcctgatcagccggatctcttctgtcggcggcgtgaagatgcacaccatgagcagctcccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 95 - RNA, 348 nt, Artificial Sequence
  RNA sequence of Cpd.15
  modified_base (1)..(348): m1f (uridine is 1-methylpseudouridine)
auguggcugcuggugucugugauccugaucagccggaucucuucugucggcggcgugaagaugcacaccaugagcagcucccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 96 - DNA, 2728 bp, Artificial Sequence
  DNA sequence of vector_ pMA-T with Cpd.15
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgtggctgctggtgtctgtgatcctgatcagccggatctcttctgtcggcggcgtgaagatgcacaccatgagcagctcccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 97 - protein, 20 aa, Homo sapiens
MVLLTAVLLLLAAYAGPAQS

SEQ ID NO: 98 - DNA, 60 bp, Artificial Sequence
  IBP5 SP
atggttctgctgacagctgttctgctgctgctggccgcttatgctggacctgctcagagc

SEQ ID NO: 99 - DNA, 354 bp, Artificial Sequence
  DNA sequence of Cpd.16
atggttctgctgacagctgttctgctgctgctggccgcttatgctggacctgctcagagcgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 100 - RNA, 354 nt, Artificial Sequence
  RNA sequence of Cpd.16
  modified_base (1)..(354): m1f (uridine is 1-methylpseudouridine)
augguucugcugacagcuguucugcugcugcuggccgcuuaugcuggaccugcucagagcgugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 101 - DNA, 2701 bp, Artificial Sequence
  DNA sequence of vector_ pMA-RQ with Cpd.16
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatggttctgctgacagctgttctgctgctgctggccgcttatgctggacctgctcagagcgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 102 - protein, 18 aa, Homo sapiens
MSILFYVIFLAYLRGIQG

SEQ ID NO: 103 - DNA, 54 bp, Artificial Sequence
  NTF3 SP
atgagcatcctgttctacgtgatcttcctggcctacctgagaggcatccagggc

SEQ ID NO: 104 - DNA, 348 bp, Artificial Sequence
  DNA sequence of Cpd.17
atgagcatcctgttctacgtgatcttcctggcctacctgagaggcatccagggcgtgaagatgcacaccatgtctagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 105 - RNA, 348 nt, Artificial Sequence
  RNA sequence of Cpd.17
  modified_base (1)..(348): m1f (uridine is 1-methylpseudouridine)
augagcauccuguucuacgugaucuuccuggccuaccugagaggcauccagggcgugaagaugcacaccaugucuagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 106 - DNA, 2808 bp, Artificial Sequence
  DNA sequence of vector_ pMA-RQ with Cpd.17
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggatcctatcatccctttgaatggaagcctccgtaaaacgacggccagttaatacgactcactatagggccaccatgagcatcctgttctacgtgatcttcctggcctacctgagaggcatccagggcgtgaagatgcacaccatgtctagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaacaggaaacagctatgacgcaaagcgttgagagtggtaacgaattcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 107 - protein, 26 aa, Homo sapiens
MLVLFLLGTVFLLCPYWGELHDPIKA

SEQ ID NO: 108 - DNA, 78 bp, Artificial Sequence
  PATE2 SP
atgctggtgctgtttctgctgggcaccgtctttctgctgtgcccttattggggcgagctgcacgatcctatcaaggcc

SEQ ID NO: 109 - DNA, 372 bp, Artificial Sequence
  DNA sequence of Cpd.18
atgctggtgctgtttctgctgggcaccgtctttctgctgtgcccttattggggcgagctgcacgatcctatcaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 110 - RNA, 372 nt, Artificial Sequence
  RNA sequence of Cpd.18
  modified_base (1)..(372): m1f (uridine is 1-methylpseudouridine)
augcuggugcuguuucugcugggcaccgucuuucugcugugcccuuauuggggcgagcugcacgauccuaucaaggccgugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 111 - DNA, 2752 bp, Artificial Sequence
  DNA sequence of vector_ pMA-T with Cpd.18
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgctggtgctgtttctgctgggcaccgtctttctgctgtgcccttattggggcgagctgcacgatcctatcaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 112 - protein, 18 aa, Homo sapiens
MLALLCSCLLLAAGASDA

SEQ ID NO: 113 - DNA, 54 bp, Artificial Sequence
  SOD3 SP
atgcttgccctgctgtgttcttgtctgctgctggctgctggcgcctctgatgcc

SEQ ID NO: 114 - DNA, 348 bp, Artificial Sequence
  DNA sequence of Cpd.19
atgcttgccctgctgtgttcttgtctgctgctggctgctggcgcctctgatgccgtgaagatgcacacaatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 115 - RNA, 348 nt, Artificial Sequence
  RNA sequence of Cpd.19
  modified_base (1)..(348): m1f (uridine is 1-methylpseudouridine)
augcuugcccugcuguguucuugucugcugcuggcugcuggcgccucugaugccgugaagaugcacacaaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 116 - DNA, 2695 bp, Artificial Sequence
  DNA sequence of vector_ pMA-RQ with Cpd.19
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgcttgccctgctgtgttcttgtctgctgctggctgctggcgcctctgatgccgtgaagatgcacacaatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 117 - protein, 22 aa, Homo sapiens
MLGALLLALAILGGLSKLHCTR

SEQ ID NO: 118 - DNA, 66 bp, Artificial Sequence
  GLR
atgcttggagcactgctgctggccctggccattcttggaggactgagcaagctgcactgcaccaga

SEQ ID NO: 119 - DNA, 360 bp, Artificial Sequence
  DNA sequence of Cpd.20
atgcttggagcactgctgctggccctggccattcttggaggactgagcaagctgcactgcaccagagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 120 - RNA, 360 nt, Artificial Sequence
  RNA sequence of Cpd.20
  modified_base (1)..(360): m1f (uridine is 1-methylpseudouridine)
augcuuggagcacugcugcuggcccuggccauucuuggaggacugagcaagcugcacugcaccagagugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 121 - DNA, 2740 bp, Artificial Sequence
  DNA sequence of vector_ pMA-T with Cpd.20
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgcttggagcactgctgctggccctggccattcttggaggactgagcaagctgcactgcaccagagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 122 - protein, 19 aa, Artificial Sequence
  IGF1-Modified SP
MLILLLPLLLFKCFCDFLK

SEQ ID NO: 123 - DNA, 57 bp, Artificial Sequence
  IGF1-Modified SP
atgctgattctgctgctgcccctgctgctgttcaagtgcttctgcgacttcctgaaa

SEQ ID NO: 124 - DNA, 351 bp, Artificial Sequence
  DNA sequence of Cpd.21
atgctgattctgctgctgcccctgctgctgttcaagtgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 125 - RNA, 351 nt, Artificial Sequence
  RNA sequence of Cpd.21
  modified_base (1)..(351): m1f (uridine is 1-methylpseudouridine)
augcugauucugcugcugccccugcugcuguucaagugcuucugcgacuuccugaaagugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 126 - DNA, 2731 bp, Artificial Sequence
  DNA sequence of vector_ pMA-T with Cpd.21
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgctgattctgctgctgcccctgctgctgttcaagtgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 127 - protein, 20 aa, Artificial Sequence
  IGF2-Modified SP
MLILLLMLVLLTFLAFASCC

SEQ ID NO: 128 - DNA, 60 bp, Artificial Sequence
  IGF2-Modified SP
atgctgatcctgctgctgatgctggtgctgctgaccttcctggccttcgccagctgctgt

SEQ ID NO: 129 - DNA, 354 bp, Artificial Sequence
  DNA sequence of Cpd.22
atgctgatcctgctgctgatgctggtgctgctgaccttcctggccttcgccagctgctgtgtgaagatgcacaccatgagcagcagccacctgttctatctggccctgtgcctgctgacattcaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 130 - RNA, 354 nt, Artificial Sequence
  RNA sequence of Cpd.22
  modified_base (1)..(354): m1f (uridine is 1-methylpseudouridine)
augcugauccugcugcugaugcuggugcugcugaccuuccuggccuucgccagcugcugugugaagaugcacaccaugagcagcagccaccuguucuaucuggcccugugccugcugacauucaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 131 - DNA, 2734 bp, Artificial Sequence
  DNA sequence of vector_ pMA-T with Cpd.22
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgctgatcctgctgctgatgctggtgctgctgaccttcctggccttcgccagctgctgtgtgaagatgcacaccatgagcagcagccacctgttctatctggccctgtgcctgctgacattcaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 132 - protein, 19 aa, Artificial Sequence
  CXCL12-Modified SP
MAVVVVLVLVLTALCLSDG

SEQ ID NO: 133 - DNA, 57 bp, Artificial Sequence
  CXCL12-Modified SP
atggccgtggtggtggtgctggtcctggtgctgacagctctgtgtctgtccgacggc

SEQ ID NO: 134 - DNA, 351 bp, Artificial Sequence
  DNA sequence of Cpd.23
atggccgtggtggtggtgctggtcctggtgctgacagctctgtgtctgtccgacggcgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 135 - RNA, 351 nt, Artificial Sequence
  RNA sequence of Cpd.23
  modified_base (1)..(351): m1f (uridine is 1-methylpseudouridine)
auggccguggugguggugcugguccuggugcugacagcucugugucuguccgacggcgugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 136 - DNA, 2731 bp, Artificial Sequence
  DNA sequence of vector_pMA-T with Cpd.23
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatggccgtggtggtggtgctggtcctggtgctgacagctctgtgtctgtccgacggcgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 137 - protein, 18 aa, Artificial Sequence
  BDNF-Modified SP
MLILFLLMVILYFGCMKA

SEQ ID NO: 138 - DNA, 54 bp, Artificial Sequence
  BDNF-Modified SP
atgctgatcctgtttctgctgatggtcatcctgtacttcggctgcatgaaggcc

SEQ ID NO: 139 - DNA, 348 bp, Artificial Sequence
  DNA sequence of Cpd.24
atgctgatcctgtttctgctgatggtcatcctgtacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 140 - RNA, 348 nt, Artificial Sequence
  RNA sequence of Cpd.24
  modified_base (1)..(348): m1f (uridine is 1-methylpseudouridine)
augcugauccuguuucugcugauggucauccuguacuucggcugcaugaaggccgugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 141 - DNA, 2728 bp, Artificial Sequence
  DNA sequence of vector_pMA-T with Cpd.24
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgctgatcctgtttctgctgatggtcatcctgtacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 142 - protein, 18 aa, Artificial Sequence
  IGF1Pro-Modified SP
MLFYLALCLLTFTSSATA

SEQ ID NO: 143 - DNA, 54 bp, Artificial Sequence
  IGF1Pro-Modified SP
atgctgttctatctggccctgtgcctgctgacctttaccagctctgctaccgcc

SEQ ID NO: 144 - DNA, 267 bp, Artificial Sequence
  DNA sequence of Cpd.25
atgctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 145 - RNA, 267 nt, Artificial Sequence
  RNA sequence of Cpd.25
  modified_base (1)..(267): m1f (uridine is 1-methylpseudouridine)
augcuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 146 - DNA, 2647 bp, Artificial Sequence
  DNA sequence of vector_pMA-T with Cpd.25
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 147 - protein, 22 aa, Artificial Sequence
  INS-Modified SP
MALWLLPLLALLALWGPDPAAA

SEQ ID NO: 148 - DNA, 66 bp, Artificial Sequence
  INS-Modified SP
atggctctgtggctgctgcctctgctggcactgcttgctctgtggggacctgatcctgccgccgct

SEQ ID NO: 149 - DNA, 360 bp, Artificial Sequence
  DNA sequence of Cpd.26
atggctctgtggctgctgcctctgctggcactgcttgctctgtggggacctgatcctgccgccgctgtgaagatgcacacaatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 150 - RNA, 360 nt, Artificial Sequence
  RNA sequence of Cpd.26
  modified_base (1)..(360): m1f (uridine is 1-methylpseudouridine)
auggcucuguggcugcugccucugcuggcacugcuugcucuguggggaccugauccugccgccgcugugaagaugcacacaaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaa

SEQ ID NO: 151 - DNA, 2740 bp, Artificial Sequence
  DNA sequence of vector_pMA-T with Cpd.26
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatggctctgtggctgctgcctctgctggcactgcttgctctgtggggacctgatcctgccgccgctgtgaagatgcacacaatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 152 - protein, 24 aa, Artificial Sequence
  Epo-Modified SP
MLVCLALLLLLSLLSLPLGLPVLG

SEQ ID NO: 153 - DNA, 72 bp, Artificial Sequence
  Epo-Modified SP
atgcttgtgtgtctggctctgctgctgctcctgagcctgctgtctctgcctctgggactgcctgttcttgga

SEQ ID NO: 154 - DNA, 573 bp, Artificial Sequence
  DNA sequence of Cpd.28
atgcttgtgtgtctggctctgctgctgctcctgagcctgctgtctctgcctctgggactgcctgttcttggagcccctcctagactgatctgcgacagcagagtgctggaaagatacctgctggaagccaaagaggccgagaacatcaccacaggctgtgccgagcactgcagcctgaacgagaatatcaccgtgcctgacaccaaagtgaacttctacgcctggaagcggatggaagtgggccagcaggctgtggaagtttggcaaggactggccctgctgagcgaagctgttctgagaggacaggctctgctcgtgaacagctctcagccttgggaacctctgcaactgcacgtggacaaggccgtgtctggcctgagaagcctgaccacactgctgagagcactgggagcccagaaagaggccatctctccacctgatgctgcctctgctgcccctctgagaaccatcaccgccgacaccttcagaaagctgttccgggtgtacagcaacttcctgcggggcaagctgaagctgtacacaggcgaggcttgcagaaccggcgacagataa

SEQ ID NO: 155 - RNA, 573 nt, Artificial Sequence
  RNA sequence of Cpd.28
  modified_base (1)..(573): m1f (uridine is 1-methylpseudouridine)
augcuugugugucuggcucugcugcugcuccugagccugcugucucugccucugggacugccuguucuuggagccccuccuagacugaucugcgacagcagagugcuggaaagauaccugcuggaagccaaagaggccgagaacaucaccacaggcugugccgagcacugcagccugaacgagaauaucaccgugccugacaccaaagugaacuucuacgccuggaagcggauggaagugggccagcaggcuguggaaguuuggcaaggacuggcccugcugagcgaagcuguucugagaggacaggcucugcucgugaacagcucucagccuugggaaccucugcaacugcacguggacaaggccgugucuggccugagaagccugaccacacugcugagagcacugggagcccagaaagaggccaucucuccaccugaugcugccucugcugccccucugagaaccaucaccgccgacaccuucagaaagcuguuccggguguacagcaacuuccugcggggcaagcugaagcuguacacaggcgaggcuugcagaaccggcgacagauaa

SEQ ID NO: 156 - DNA, 2953 bp, Artificial Sequence
  DNA sequence of vector_pMA-T with Cpd.28
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgcttgtgtgtctggctctgctgctgctcctgagcctgctgtctctgcctctgggactgcctgttcttggagcccctcctagactgatctgcgacagcagagtgctggaaagatacctgctggaagccaaagaggccgagaacatcaccacaggctgtgccgagcactgcagcctgaacgagaatatcaccgtgcctgacaccaaagtgaacttctacgcctggaagcggatggaagtgggccagcaggctgtggaagtttggcaaggactggccctgctgagcgaagctgttctgagaggacaggctctgctcgtgaacagctctcagccttgggaacctctgcaactgcacgtggacaaggccgtgtctggcctgagaagcctgaccacactgctgagagcactgggagcccagaaagaggccatctctccacctgatgctgcctctgctgcccctctgagaaccatcaccgccgacaccttcagaaagctgttccgggtgtacagcaacttcctgcggggcaagctgaagctgtacacaggcgaggcttgcagaaccggcgacagataaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 157 - DNA, 555 bp, Artificial Sequence
  DNA sequence of Cpd.29
atgaccatcctgtttctgacaatggtcatcagctacttcggctgcatgaaggccgctcctccaagactgatctgcgacagcagagtgctggaaagatacctgctggaagccaaagaggccgagaacatcaccacaggctgtgccgagcactgcagcctgaacgagaatatcaccgtgcctgacaccaaagtgaacttctacgcctggaagcggatggaagtgggccagcaggctgtggaagtttggcaaggactggccctgctgtctgaggctgttctgagaggacaggctctgctggtcaacagctctcagccttgggaacctctgcaactgcacgtggacaaggccgtgtctggcctgagaagcctgaccacactgctgagagcactgggagcccagaaagaggccatctctccacctgatgctgcctctgctgcccctctgagaaccatcaccgccgacaccttcagaaagctgttccgggtgtacagcaacttcctgcggggcaagctgaagctgtacacaggcgaggcttgcagaaccggcgacagataa

SEQ ID NO: 158 - RNA, 555 nt, Artificial Sequence
  RNA sequence of Cpd.29
  modified_base (1)..(555): m1f (uridine is 1-methylpseudouridine)
augaccauccuguuucugacaauggucaucagcuacuucggcugcaugaaggccgcuccuccaagacugaucugcgacagcagagugcuggaaagauaccugcuggaagccaaagaggccgagaacaucaccacaggcugugccgagcacugcagccugaacgagaauaucaccgugccugacaccaaagugaacuucuacgccuggaagcggauggaagugggccagcaggcuguggaaguuuggcaaggacuggcccugcugucugaggcuguucugagaggacaggcucugcuggucaacagcucucagccuugggaaccucugcaacugcacguggacaaggccgugucuggccugagaagccugaccacacugcugagagcacugggagcccagaaagaggccaucucuccaccugaugcugccucugcugccccucugagaaccaucaccgccgacaccuucagaaagcuguuccggguguacagcaacuuccugcggggcaagcugaagcuguacacaggcgaggcuugcagaaccggcgacagauaa

SEQ ID NO: 159 - DNA, 2902 bp, Artificial Sequence
  DNA sequence of vector_pMA-RQ with Cpd.29
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgaccatcctgtttctgacaatggtcatcagctacttcggctgcatgaaggccgctcctccaagactgatctgcgacagcagagtgctggaaagatacctgctggaagccaaagaggccgagaacatcaccacaggctgtgccgagcactgcagcctgaacgagaatatcaccgtgcctgacaccaaagtgaacttctacgcctggaagcggatggaagtgggccagcaggctgtggaagtttggcaaggactggccctgctgtctgaggctgttctgagaggacaggctctgctggtcaacagctctcagccttgggaacctctgcaactgcacgtggacaaggccgtgtctggcctgagaagcctgaccacactgctgagagcactgggagcccagaaagaggccatctctccacctgatgctgcctctgctgcccctctgagaaccatcaccgccgacaccttcagaaagctgttccgggtgtacagcaacttcctgcggggcaagctgaagctgtacacaggcgaggcttgcagaaccggcgacagataactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 160 - DNA, 327 bp, Artificial Sequence
  DNA sequence of Cpd.31
atggcactttggctgctgcctcttctggctctgctggctctttggggacctgatcctgccgccgctttcgtgaatcagcacctgtgtggcagccacctggtggaagccctgtatctcgtgtgtggcgagcggggcttcttctacacccctaagacaagaagagaggccgaggacctgcaagtcggccaggttgaacttggcggaggacctggtgctggatctctgcaacctctggcactggaaggcagcctgcagaagaggggcatcgtcgagcagtgctgcaccagcatctgtagcctgtaccagctggaaaactactgcaactga

SEQ ID NO: 161 - RNA, 327 nt, Artificial Sequence
  RNA sequence of Cpd.31
  modified_base (1)..(327): m1f (uridine is 1-methylpseudouridine)
auggcacuuuggcugcugccucuucuggcucugcuggcucuuuggggaccugauccugccgccgcuuucgugaaucagcaccuguguggcagccaccugguggaagcccuguaucucguguguggcgagcggggcuucuucuacaccccuaagacaagaagagaggccgaggaccugcaagucggccagguugaacuuggcggaggaccuggugcuggaucucugcaaccucuggcacuggaaggcagccugcagaagaggggcaucgucgagcagugcugcaccagcaucuguagccuguaccagcuggaaaacuacugcaacuga

SEQ ID NO: 162 - DNA, 2707 bp, Artificial Sequence
  DNA sequence of vector_pMA-T with Cpd.31
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatggcactttggctgctgcctcttctggctctgctggctctttggggacctgatcctgccgccgctttcgtgaatcagcacctgtgtggcagccacctggtggaagccctgtatctcgtgtgtggcgagcggggcttcttctacacccctaagacaagaagagaggccgaggacctgcaagtcggccaggttgaacttggcggaggacctggtgctggatctctgcaacctctggcactggaaggcagcctgcagaagaggggcatcgtcgagcagtgctgcaccagcatctgtagcctgtaccagctggaaaactactgcaactgaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 163 - DNA, 315 bp, Artificial Sequence
  DNA sequence of Cpd.32
atgaccatcctgtttctgacaatggtcatcagctacttcggctgcatgaaggcctttgtgaaccagcacctgtgcggcagccatctggtggaagccctgtatctcgtgtgtggcgagcggggcttcttctacacccctaagacaagaagagaggccgaggacctgcaagtcggccaggttgaacttggcggaggacctggtgctggatctctgcaacctctggctctggaaggcagcctgcagaagaggggcatcgtcgagcagtgctgcaccagcatctgtagcctgtaccagctggaaaactactgcaactga

SEQ ID NO: 164 - RNA, 315 nt, Artificial Sequence
  RNA sequence of Cpd.32
  modified_base (1)..(315): m1f (uridine is 1-methylpseudouridine)
augaccauccuguuucugacaauggucaucagcuacuucggcugcaugaaggccuuugugaaccagcaccugugcggcagccaucugguggaagcccuguaucucguguguggcgagcggggcuucuucuacaccccuaagacaagaagagaggccgaggaccugcaagucggccagguugaacuuggcggaggaccuggugcuggaucucugcaaccucuggcucuggaaggcagccugcagaagaggggcaucgucgagcagugcugcaccagcaucuguagccuguaccagcuggaaaacuacugcaacuga

SEQ ID NO: 165 - DNA, 2662 bp, Artificial Sequence
  DNA sequence of vector_ pMA-RQ with Cpd.32
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgaccatcctgtttctgacaatggtcatcagctacttcggctgcatgaaggcctttgtgaaccagcacctgtgcggcagccatctggtggaagccctgtatctcgtgtgtggcgagcggggcttcttctacacccctaagacaagaagagaggccgaggacctgcaagtcggccaggttgaacttggcggaggacctggtgctggatctctgcaacctctggctctggaaggcagcctgcagaagaggggcatcgtcgagcagtgctgcaccagcatctgtagcctgtaccagctggaaaactactgcaactgactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 166 - protein, 20 aa, Artificial Sequence
  IL4-Modified SP
MLLLPPLFFLLACAGNFVHG

SEQ ID NO: 167 - DNA, 60 bp, Artificial Sequence
  IL4-Modified SP
atgttgttgctgcctccactgttctttctgctggcctgcgccggcaattttgtgcacggc

SEQ ID NO: 168 - DNA, 450 bp, Artificial Sequence
  DNA sequence of Cpd.34
atgttgttgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctga

SEQ ID NO: 169 - RNA, 450 nt, Artificial Sequence
  RNA sequence of Cpd.34
  modified_base (1)..(450): m1f (uridine is 1-methylpseudouridine)
auguuguugcugccuccacuguucuuucugcuggccugcgccggcaauuuugugcacggccacaagugcgacaucacccugcaagagaucaucaagacccugaacagccugaccgagcagaaaacccugugcaccgagcugaccgugaccgauaucuuugccgccagcaagaacacaaccgagaaagagacauucugcagagccgccaccgugcugagacaguucuacagccaccacgagaaggacaccagaugccugggagcuacagcccagcaguuccacagacacaagcagcugauccgguuccugaagcggcuggacagaaaucuguggggacucgccggccugaauagcugcccugugaaagaggccaaccagucuacccuggaaaacuuccuggaacggcugaaaaccaucaugcgcgagaaguacagcaagugcagcagcuga

SEQ ID NO: 170 - DNA, 2830 bp, Artificial Sequence
  DNA sequence of vector_ pMA-T with Cpd.34
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgttgttgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 171 - DNA, 450 bp, Artificial Sequence
  DNA sequence of Cpd.35
atgagcctgagctttctgctgctgctgttcttcagccacctgatcctgtctgcctgggctcacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctactcccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctga

SEQ ID NO: 172 - RNA, 450 nt, Artificial Sequence
  RNA sequence of Cpd.35
  modified_base (1)..(450): m1f (uridine is 1-methylpseudouridine)
augagccugagcuuucugcugcugcuguucuucagccaccugauccugucugccugggcucacaagugcgacaucacccugcaagagaucaucaagacccugaacagccugaccgagcagaaaacccugugcaccgagcugaccgugaccgauaucuuugccgccagcaagaacacaaccgagaaagagacauucugcagagccgccaccgugcugagacaguucuacucccaccacgagaaggacaccagaugccugggagcuacagcccagcaguuccacagacacaagcagcugauccgguuccugaagcggcuggacagaaaucuguggggacucgccggccugaauagcugcccugugaaagaggccaaccagucuacccuggaaaacuuccuggaacggcugaaaaccaucaugcgcgagaaguacagcaagugcagcagcuga

SEQ ID NO: 173 - DNA, 2797 bp, Artificial Sequence
  DNA sequence of vector_ pMA-RQ with Cpd.35
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgagcctgagctttctgctgctgctgttcttcagccacctgatcctgtctgcctgggctcacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctactcccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 174 - protein, 18 aa, Artificial Sequence
  IL10-Modified SP
MVLLALLCCLVLLTGVRA

SEQ ID NO: 175 - DNA, 54 bp, Artificial Sequence
  IL10-Modified SP
atggttctgctggctctgctgtgttgtctggtgctgctgacaggcgtcagagcc

SEQ ID NO: 176 - DNA, 537 bp, Artificial Sequence
  DNA sequence of Cpd.37
atggttctgctggctctgctgtgttgtctggtgctgctgacaggcgtcagagcctctcctggacagggaacccagagcgagaatagctgcacccactttccaggcaacctgcctaacatgctgcgggacctgagagatgccttcagcagagtgaaaacattcttccagatgaaggaccagctggacaacctgctgctgaaagagagcctgctggaagatttcaagggctacctgggctgtcaggccctgagcgagatgatccagttctacctggaagaagtgatgccccaggccgagaatcaggaccccgatattaaggcccacgtgaacagcctgggcgagaacctgaaaaccctgcggctgagactgcggcggtgccacagatttctgccctgcgagaacaagagcaaggccgtggaacaagtgaagaacgccttcaacaagctgcaagagaagggcatctacaaggccatgtccgagttcgacatcttcatcaactacatcgaggcctacatgaccatgaagatccggaactga

SEQ ID NO: 177 - RNA, 537 nt, Artificial Sequence
  RNA sequence of Cpd.37
  modified_base (1)..(537): m1f (uridine is 1-methylpseudouridine)
augguucugcuggcucugcuguguugucuggugcugcugacaggcgucagagccucuccuggacagggaacccagagcgagaauagcugcacccacuuuccaggcaaccugccuaacaugcugcgggaccugagagaugccuucagcagagugaaaacauucuuccagaugaaggaccagcuggacaaccugcugcugaaagagagccugcuggaagauuucaagggcuaccugggcugucaggcccugagcgagaugauccaguucuaccuggaagaagugaugccccaggccgagaaucaggaccccgauauuaaggcccacgugaacagccugggcgagaaccugaaaacccugcggcugagacugcggcggugccacagauuucugcccugcgagaacaagagcaaggccguggaacaagugaagaacgccuucaacaagcugcaagagaagggcaucuacaaggccauguccgaguucgacaucuucaucaacuacaucgaggccuacaugaccaugaagauccggaacuga

SEQ ID NO: 178 - DNA, 2917 bp, Artificial Sequence
  DNA sequence of vector_ pMA-T with Cpd.37
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatggttctgctggctctgctgtgttgtctggtgctgctgacaggcgtcagagcctctcctggacagggaacccagagcgagaatagctgcacccactttccaggcaacctgcctaacatgctgcgggacctgagagatgccttcagcagagtgaaaacattcttccagatgaaggaccagctggacaacctgctgctgaaagagagcctgctggaagatttcaagggctacctgggctgtcaggccctgagcgagatgatccagttctacctggaagaagtgatgccccaggccgagaatcaggaccccgatattaaggcccacgtgaacagcctgggcgagaacctgaaaaccctgcggctgagactgcggcggtgccacagatttctgccctgcgagaacaagagcaaggccgtggaacaagtgaagaacgccttcaacaagctgcaagagaagggcatctacaaggccatgtccgagttcgacatcttcatcaactacatcgaggcctacatgaccatgaagatccggaactgaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 179 - DNA, 537 bp, Artificial Sequence
  DNA sequence of Cpd.38
atgaccatcctgtttctgacaatggtcatcagctacttcggctgcatgaaggcctctccaggccagggaacacagagcgagaatagctgcacccactttccaggcaacctgcctaacatgctgcgggacctgagagatgccttctccagagtgaaaacattcttccagatgaaggaccagctggacaacctgctgctgaaagagagcctgctggaagatttcaagggctacctgggctgtcaggccctgagcgagatgatccagttctacctggaagaagtgatgccccaggccgagaatcaggaccccgatattaaggcccacgtgaacagcctgggcgagaacctgaaaaccctgcggctgagactgcggcggtgccacagatttctgccctgcgagaacaagagcaaggccgtggaacaagtgaagaacgccttcaacaagctgcaagagaagggcatctacaaggccatgtccgagttcgacatcttcatcaactacatcgaggcctacatgaccatgaagatccggaactga

SEQ ID NO: 180 - RNA, 537 nt, Artificial Sequence
  RNA sequence of Cpd.38
  modified_base (1)..(537): m1f (uridine is 1-methylpseudouridine)
augaccauccuguuucugacaauggucaucagcuacuucggcugcaugaaggccucuccaggccagggaacacagagcgagaauagcugcacccacuuuccaggcaaccugccuaacaugcugcgggaccugagagaugccuucuccagagugaaaacauucuuccagaugaaggaccagcuggacaaccugcugcugaaagagagccugcuggaagauuucaagggcuaccugggcugucaggcccugagcgagaugauccaguucuaccuggaagaagugaugccccaggccgagaaucaggaccccgauauuaaggcccacgugaacagccugggcgagaaccugaaaacccugcggcugagacugcggcggugccacagauuucugcccugcgagaacaagagcaaggccguggaacaagugaagaacgccuucaacaagcugcaagagaagggcaucuacaaggccauguccgaguucgacaucuucaucaacuacaucgaggccuacaugaccaugaagauccggaacuga

SEQ ID NO: 181 - DNA, 2884 bp, Artificial Sequence
  DNA sequence of vector_ pMA-RQ with Cpd.38
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgaccatcctgtttctgacaatggtcatcagctacttcggctgcatgaaggcctctccaggccagggaacacagagcgagaatagctgcacccactttccaggcaacctgcctaacatgctgcgggacctgagagatgccttctccagagtgaaaacattcttccagatgaaggaccagctggacaacctgctgctgaaagagagcctgctggaagatttcaagggctacctgggctgtcaggccctgagcgagatgatccagttctacctggaagaagtgatgccccaggccgagaatcaggaccccgatattaaggcccacgtgaacagcctgggcgagaacctgaaaaccctgcggctgagactgcggcggtgccacagatttctgccctgcgagaacaagagcaaggccgtggaacaagtgaagaacgccttcaacaagctgcaagagaagggcatctacaaggccatgtccgagttcgacatcttcatcaactacatcgaggcctacatgaccatgaagatccggaactgactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 182 - DNA, 66 bp, Artificial Sequence
  INS-Modified SP of Cpd.31
atggcactttggctgctgcctcttctggctctgctggctctttggggacctgatcctgccgccgct

SEQ ID NO: 183 - DNA, 60 bp, Artificial Sequence
  FGF5 SP of Cpd.35
atgagcctgagctttctgctgctgctgttcttcagccacctgatcctgtctgcctgggct

SEQ ID NO: 184 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 185 - protein, 82 aa, Homo sapiens
FVNQHLCGSHLVEALYLVCGERGFFYTPKTEAEDLQVGQVELGGGPGAGSLQPLALEGSLQGIVEQCCTSICSLYQLENYCN

SEQ ID NO: 186 - protein, 129 aa, Homo sapiens [sequence omitted]

SEQ ID NO: 187 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 188 - protein, 97 aa, Homo sapiens
VKMHTMSSSHLFYLALCLLTFTSSATAGPETLCGAELVDALQFVCGDRGFYFNKPTGYGSSSRRAPQTGIVDECCFRSCDLRRLEMYCAPLKPAKSA

SEQ ID NO: 189 - protein, 21 aa, Artificial Sequence
  ALPI-Modified SP
MLLVLLLLALLLLLLGASAAP

SEQ ID NO: 190 - DNA, 63 bp, Artificial Sequence
  ALPI-Modified SP
atgctgcttgttctgctgctgctggctctgctgctcctgcttcttggagcttctgccgctcct

SEQ ID NO: 191 - DNA, 357 bp, Artificial Sequence
  DNA sequence of Cpd.39
atgctgcttgttctgctgctgctggctctgctgctcctgcttcttggagcttctgccgctcctgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagctcctctagaagggctcctcagaccggaatcgtggacgagtgctgcttcagaagctgcgacctgcggcggctggaaatgtattgtgcccctctgaagcctgccaagagcgcctaa

SEQ ID NO: 192 - RNA, 357 nt, Artificial Sequence
  RNA sequence of Cpd.39
  modified_base (1)..(357): m1f (uridine is 1-methylpseudouridine)
augcugcuuguucugcugcugcuggcucugcugcuccugcuucuuggagcuucugccgcuccugugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcuccucuagaagggcuccucagaccggaaucguggacgagugcugcuucagaagcugcgaccugcggcggcuggaaauguauugugccccucugaagccugccaagagcgccuaa

SEQ ID NO: 193 - DNA, 2737 bp, Artificial Sequence
  DNA sequence of vector_ pMA-T with Cpd.39
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgctgcttgttctgctgctgctggctctgctgctcctgcttcttggagcttctgccgctcctgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagctcctctagaagggctcctcagaccggaatcgtggacgagtgctgcttcagaagctgcgacctgcggcggctggaaatgtattgtgcccctctgaagcctgccaagagcgcctaaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

(19) United States
(12) Patent Application Publication
(10) Pub. No.: US 2022/0002364 A1
(43) Pub. Date: Jan. 6, 2022

(54) RNA ENCODING A PROTEIN

(71) Applicant: Versameb AG, Basel (CH)

(72) Inventors: Justin Antony SELVARAJ, Weil am Rhein (DE); Hervé SCHAFFHAUSER, Habsheim (FR); Friedrich METZGER, Freiburg (DE)

(21) Appl. No.: 17/348,876

(22) Filed: Jun. 16, 2021

Related U.S. Application Data
(63) Continuation of application No. PCT/EP2019/086019, filed on Dec. 18, 2019.

(30) Foreign Application Priority Data
Dec. 19, 2018 (EP) 18214221.6
Nov. 8, 2019 (EP) 19208066.1

(51) Int. Cl. C07K 14/475; C12N 15/62; A61P 21/00

(52) U.S. Cl. CPC C07K 14/475; C12N 15/625; A61K 48/00; C07K 2319/02; A61P 21/00

(57) ABSTRACT

The present invention relates to a mRNA comprising a nucleic acid sequence encoding a protein and a signal peptide and a transcription unit, an expression vector or a gene therapy vector comprising a nucleic acid encoding a protein and a signal peptide. Also disclosed herein is a therapeutic composition comprising said mRNA, transcription unit, expression vector or gene therapy vector and use of the therapeutic composition in treating a disease or a condition.

### CROSS-REFERENCE TO RELATED APPLICATIONS

This application is a continuation of International Application No. PCT/EP2019/086019, filed Dec. 18, 2019, which claims the benefit of European Patent Application No. 18214221.6, filed Dec. 19, 2018 and European Patent Application No. 19208066.1, filed Nov. 8, 2019, each of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Jun. 23, 2021, is named 57623_702_301_SL.txt and is 230,555 bytes in size.

### THE FIELD OF THE INVENTION

The present invention relates to a mRNA comprising a nucleic acid sequence encoding a protein and a signal peptide, wherein the amino acids 1-9 of the N-terminal end of the amino acid sequence of the signal peptide have an average hydrophobic score of above 2, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

The present invention further relates to a mRNA comprising a nucleic acid sequence encoding i) a protein; and ii) a signal peptide heterologous to said protein, wherein the signal peptide heterologous to said protein is the signal peptide of the brain-derived neurotrophic factor (BDNF) and wherein the protein is not an oxidoreductase. The present invention also relates to a transcription unit or expression vector comprising a nucleic acid sequence encoding a protein and a signal peptide, wherein the amino acids 1-9 of the N-terminal end of the amino acid sequence of the signal peptide have an average hydrophobic score of above 2, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid. The present invention also relates to a transcription unit or expression vector comprising a nucleic acid sequence encoding a protein and a signal peptide heterologous to said protein, wherein the signal peptide heterologous to said protein is the signal peptide of the brain-derived neurotrophic factor (BDNF). The present invention also relates to a therapeutic composition and a kit comprising the mRNA, and/or the transcription unit or expression vector. The present invention also relates to a mRNA, a transcription unit or expression vector, a therapeutic composition and/or a kit for use as a medicament in particular to a mRNA comprising a nucleic acid sequence encoding i) IGF1; and ii) the signal peptide of the brain-derived neurotrophic factor (BDNF) for use as a medicament. The present invention also relates to a mRNA and a therapeutic composition thereof for use in a method of treating skeletal muscle injury.

### BACKGROUND OF THE INVENTION

Different attempts have been made in the past to increase the yield of the expression and secretion of an encoded protein, in particular by use of improved expression systems, both in vitro and/or in vivo. Methods for increasing expression and secretion described generally in the prior art are conventionally based on the use of expression vectors or cassettes containing specific promoters and corresponding regulation elements. As these expression vectors or cassettes are typically limited to particular cell systems, these expression systems have to be adapted for use in different cell systems. Such adapted expression vectors or cassettes are then usually transfected into the cells and typically treated in dependence of the specific cell line. Therefore, preference is given primarily to those nucleic acid molecules like mRNA which are able to express the encoded proteins in a target cell by systems inherent in the cell, independent of promoters and regulation elements which are specific for particular cell types. In this context, there can be distinguished between mRNA stabilizing elements and elements which increase translation efficiency of the mRNA. For example, WO 02/098443 describes mRNAs that are stabilised in general form and optimised for translation in their coding regions. WO 02/098443 further discloses a method for determining sequence modifications. WO 02/098443 additionally describes possibilities for substituting adenine and uracil nucleotides in mRNA sequences in order to increase the guanine/cytosine (G/C) content of the sequences. In this context, WO 02/098443 generally mentions sequences as a base sequence for such modifications, in which the modified mRNA codes for at least one biologically active peptide or polypeptide, which is translated in the patient to be treated, for example, either not at all or inadequately or with faults. In a further approach to increase the expression of an encoded protein the application WO 2007/036366 describes the positive effect of long poly(A) sequences (particularly longer than 120 bp) and the combination of at least two 3′ untranslated regions of the beta globin gene on mRNA stability and translational activity. Despite of all progress in the art, efficient expression and in particular efficient secretion of an encoded protein in cell-free systems, cells or organisms (recombinant expression) is still a challenging problem.

### SUMMARY OF THE INVENTION

The present invention provides a mRNA comprising a nucleic acid sequence encoding a protein and a signal peptide, wherein the amino acids 1-9 of the N-terminal end of the amino acid sequence of the signal peptide have an average hydrophobic score of above 2, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

The present invention further provides a mRNA comprising a nucleic acid sequence encoding

i) a protein; and
ii) a signal peptide heterologous to said protein,
wherein the signal peptide heterologous to said protein is the signal peptide of the brain-derived neurotrophic factor (BDNF) and wherein the protein is not an oxidoreductase, in particular the present invention provides a mRNA comprising a nucleic acid sequence encoding
i) a protein; and
ii) a signal peptide heterologous to said protein,
wherein the signal peptide heterologous to said protein is the signal peptide of the brain-derived neurotrophic factor (BDNF) and wherein the protein is selected from the group consisting of carboxypeptidases; cytokines; extracellular ligands and transporters; extracellular matrix proteins; glucosidases; glycosyltransferases; growth factors; growth factor binding proteins; heparin binding proteins; hormones; hydrolases; immunoglobulins; isomerases; kinases; lyases; metalloenzyme inhibitors; metalloproteases; milk proteins; neuroactive proteins; proteases; protease inhibitors; protein phosphatases; esterases; transferases; and vasoactive proteins.

The present invention further provides a transcription unit or expression vector comprising a nucleic acid sequence encoding a protein and a signal peptide wherein the amino acids 1-9 of the N-terminal end of the amino acid sequence of the signal peptide have an average hydrophobic score of above 2, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

The present invention further provides a transcription unit or expression vector comprising a nucleic acid sequence encoding a protein and a signal peptide heterologous to said protein, wherein the signal peptide heterologous to said protein is the signal peptide of the brain-derived neurotrophic factor (BDNF) and wherein the protein is not an oxidoreductase. The present invention further provides a transcription unit or expression vector comprising a nucleic acid sequence encoding a protein and a signal peptide heterologous to said protein, wherein the signal peptide heterologous to said protein is the signal peptide of the brain-derived neurotrophic factor (BDNF), wherein the protein is selected from the group consisting of carboxypeptidases; cytokines; extracellular ligands and transporters; extracellular matrix proteins; glucosidases; glycosyltransferases; growth factors; growth factor binding proteins; heparin binding proteins; hormones; hydrolases; immunoglobulins; isomerases; kinases; lyases; metalloenzyme inhibitors; metalloproteases; milk proteins; neuroactive proteins; proteases; protease inhibitors; protein phosphatases; esterases; transferases; and vasoactive proteins. The present invention further provides a therapeutic composition comprising the mRNA and/or the transcription unit or expression vector mentioned above. The present invention further provides a kit comprising the mRNA, the transcription unit or expression vector and/or the therapeutic composition mentioned above, and instructions, optionally a vector map, optionally a host cell, optionally a cultivation medium for the cultivation of a host cell, and/or optionally a selection medium for selecting and cultivating a transfected host cell. The present invention further provides the mRNA, the transcription unit or expression vector, the therapeutic composition or the kit mentioned above for use as a medicament. The present invention further provides a mRNA comprising a nucleic acid sequence encoding i) IGF1; and ii) the signal peptide of the brain-derived neurotrophic factor (BDNF) for use as a medicament. The present invention further provides a mRNA or a therapeutic composition containing mRNA for use in a method of treating skeletal muscle injury.

The present inventors have surprisingly found that a mRNA comprising a nucleic acid sequence encoding a protein and a signal peptide, wherein the amino acids 1-9 of the N-terminal end of the amino acid sequence of the signal peptide have an average hydrophobic score of above 2, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid,
provides for more efficient secretion of the protein by cells transfected with this mRNA compared to the secretion of the protein by cells transfected with a mRNA encoding the protein and its natural, homologous signal peptide. In particular the present inventors have surprisingly found that a mRNA comprising a nucleic acid sequence encoding i) a protein; and ii) a BDNF signal peptide heterologous to said protein, provides for more efficient secretion of the protein by cells transfected with this mRNA than the natural, homologous signal peptide of the protein. The amount of secreted protein is up to six times higher when compared with mRNA comprising the same protein and the natural, homologous signal peptide of the protein. This unexpected finding is very useful to effectively deliver and express mRNA encoding a desired protein in a cell, and to obtain higher amounts of secreted protein than with the natural, homologous signal peptide of the protein. The higher amounts of secreted protein obtained with the same amount of mRNA provided by the present invention are extremely useful for lowering the therapeutic dose that needs to be applied locally into a tissue, thereby increasing its safety window against potential mRNA-related side effects. Furthermore, it makes the application more amenable to formulations for controlled release and device coatings. Furthermore, it reduces the mRNA-related immunogenicity risk and it makes the application more amenable to tissues where limited volumes can be injected or for previously unaccessible tissues. The present inventors have also found that it is possible to effectively deliver and express mRNA, in particular mRNA encoding human IGF-1 to skeletal muscles thereby allowing the expression of the desired polypeptide in the skeletal muscles to provide a relevant functional benefit to the muscle. The mRNA is preferably present in a liquid composition, preferably in naked form. This liquid composition can be delivered directly to skeletal muscles, e.g. by injection, and there is no need for any gene transfer vectors or carriers for the mRNA or methods for enhancing the transfer into the tissue like electrotransfer or ultrasound. Moreover, the injection of mRNA into injured skeletal muscles was shown to accelerate the recovery process and result in an increase of the function of the skeletal muscles. Surprisingly animals treated with mRNA encoding IGF-1 reached functional levels in the healthy range by 16 days. In contrast, control animals treated with vehicle did not even achieve full functional recovery by day 28.

### BRIEF DESCRIPTION OF THE FIGURES

FIGS. 1A-1B show DNA and RNA sequence of Cpd.1. FIG. 1A shows the DNA sequence (SEQ ID NO:1) of human codon-optimized IGF1 containing its pre-, pro- and coding domains. The sequence for the pre-domain (signalling peptide) is indicated in italic, the sequence for the pro-domain is underlined, the IGF-I coding domain is indicated in bold and the stop codon in bold and underlined. FIG. 1B illustrates the RNA sequence of pre-, pro- and coding domain of human IGF1 (SEQ ID NO:2), wherein uridine is N1-Methylpseudouridine. Pre- and pro-domains are cleaved of upon secretion.

FIGS. 2A-2B show DNA and RNA sequence of Cpd.2. FIG. 2A shows the DNA sequence (SEQ ID NO:3) of human codon-optimized IGF1 containing the IGF2 pre-domain and IGF1 pro- and coding domains. The sequence for the pre-domain (signalling peptide) is indicated in italic, the sequence for the pro-domain is underlined, the IGF1 coding domain is indicated in bold and the stop codon in bold and underlined. FIG. 2B illustrates the RNA sequence of IGF2 pre- and IGF1 pro- and coding domains (SEQ ID NO:4), wherein uridine is N1-Methylpseudouridine. Pre- and pro-domains are cleaved of upon secretion.

FIGS. 3A-3B show DNA and RNA sequence of Cpd.3. FIG. 3A shows the DNA sequence (SEQ ID NO:5) of human codon-optimized IGF1 containing the ALB pre-domain and IGF1 pro- and coding domains. The sequence for the pre-domain (signalling peptide) is indicated in italic, the sequence for the pro-domain is underlined, the IGF1 coding domain is indicated in bold and the stop codon in bold and underlined. FIG. 3B illustrates the RNA sequence of ALB pre- and IGF1 pro- and coding domains (SEQ ID NO:6), wherein uridine is N1-Methylpseudouridine. Pre- and pro-domains are cleaved of upon secretion.

FIGS. 4A-4B show DNA and RNA sequence of Cpd.4. FIG. 4A shows the DNA sequence (SEQ ID NO:7) of human codon-optimized IGF1 containing the BDNF pre-domain and IGF1 pro- and coding domains. The sequence for the pre-domain (signalling peptide) is indicated in italic, the sequence for the pro-domain is underlined, the IGF1 coding domain is indicated in bold and the stop codon in bold and underlined. FIG. 4B illustrates the RNA sequence of BDNF pre- and IGF1 pro- and coding domains (SEQ ID NO:8), wherein uridine is N1-Methylpseudouridine. Pre- and pro-domains are cleaved of upon secretion.

FIGS. 5A-5B show DNA and RNA sequence of Cpd.5. FIG. 5A shows the DNA sequence (SEQ ID NO:9) of human codon-optimized IGF1 containing the CXCL12 pre-domain and IGF1 pro- and coding domains. The sequence for the pre-domain (signalling peptide) is indicated in italic, the sequence for the pro-domain is underlined, the IGF1 coding domain is indicated in bold and the stop codon in bold and underlined. FIG. 5B illustrates the RNA sequence of CXCL12 pre- and IGF1 pro- and coding domains (SEQ ID NO:10), wherein uridine is N1-Methylpseudouridine. Pre- and pro-domains are cleaved of upon secretion.

FIGS. 6A-6B show DNA and RNA sequence of Cpd.6. FIG. 6A shows the DNA sequence (SEQ ID NO:11) of human codon-optimized IGF1 containing the synthetic signaling peptide 1 pre-domain and IGF1 pro- and coding domains. The sequence for the pre-domain (signalling peptide) is indicated in italic, the sequence for the pro-domain is underlined, the IGF1 coding domain is indicated in bold and the stop codon in bold and underlined. FIG. 6B illustrates the RNA sequence of synthetic signaling peptide 1 pre- and IGF1 pro- and coding domains (SEQ ID NO:12), wherein uridine is N1-Methylpseudouridine. Pre- and pro-domains are cleaved of upon secretion.

FIGS. 7A-7B show DNA and RNA sequence of Cpd.7. FIG. 7A shows the DNA sequence (SEQ ID NO:13) of human codon-optimized IGF1 containing the synthetic signaling peptide 2 pre-domain and IGF1 pro- and coding domains. The sequence for the pre-domain (signalling peptide) is indicated in italic, the sequence for the pro-domain is underlined, the IGF1 coding domain is indicated in bold and the stop codon in bold and underlined. FIG. 7B illustrates the RNA sequence of synthetic signaling peptide 2 pre- and IGF1 pro- and coding domains (SEQ ID NO:14), wherein uridine is N1-Methylpseudouridine. Pre- and pro-domains are cleaved of upon secretion.

FIG. 8 shows the DNA sequence of vector pVAX.A120 with Cpd.1 insert marked in bold and underlined (SEQ ID NO:15). The ORFs of Cpd.1 was digested from its original plasmid and subcloned into the vector.

FIG. 9 shows the DNA sequence of vector pMA-T with Cpd.2 insert marked in bold and underlined (SEQ ID NO:16). The ORFs of Cpd.2 was digested from its original plasmid and subcloned into the vector.

FIG. 10 shows the DNA sequence of vector pMA-T with Cpd.3 insert marked in bold and underlined (SEQ ID NO:17). The ORFs of Cpd.3 was digested from its original plasmid and subcloned into the vector.

FIG. 11 shows the DNA sequence of vector pMA-T with Cpd.4 insert marked in bold and underlined (SEQ ID NO:18). The ORFs of Cpd.4 was digested from its original plasmid and subcloned into the vector.

FIG. 12 shows the DNA sequence of vector pMA-T with Cpd.5 insert marked in bold and underlined (SEQ ID NO:19). The ORFs of Cpd.5 was digested from its original plasmid and subcloned into the vector.

FIG. 13 shows the DNA sequence of vector pMA-RQ with Cpd.6 marked in bold and underlined (SEQ ID NO:20). The ORFs of Cpd.6 was digested from its original plasmid and subcloned into the vector.

FIG. 14 shows the DNA sequence of vector pMA-RQ with Cpd.7 marked in bold and underlined (SEQ ID NO:21). The ORFs of Cpd.7 was digested from its original plasmid and subcloned into the vector.

FIG. 15 shows the forward (SEQ ID NO:22) and reverse primer (SEQ ID NO:23) sequences used to amplify pMA-T and pMA-RQ plasmids for the IVT of mRNAs.

FIG. 16 shows the gene names, UniProt numbers, codon-optimized DNA and amino acid sequences and vectors of the signal peptides of Cpd.1-Cpd.7 (SEQ ID Nos: 24-37). Note that the signal peptides of Cpd.6 and Cpd.7 are synthetic peptides and not matching known protein sequences in the public databases.

FIG. 17 shows the induction of IGF1 secretion from human embryonic kidney cells (HEK293T) by mRNA transfection with Cpd.1-Cpd.7. HEK293T cells were transfected with each 2 μg Cp.1-Cpd.7, and secreted IGF1 was measured after 24 hours in the cell culture supernatant using a specific ELISA. Cpd.4 induced IGF1 secretion significantly higher than Cpd.1 (3.3-fold). Data represent means±standard error of the mean of 4 replicates. Significance (***, <0.001) was assessed by one-way ANOVA followed by Dunnett's multiple comparison test.

FIG. 18 shows the concentration-dependence of the induction of IGF1 secretion in HEK293T cells after mRNA transfection with Cpd.1 or Cpd.4. Cells were transfected with Cpd.1 or Cpd.4 at different concentrations (0, 0.02, 0.06, 0.2, 0.6 or 2 μg), and secreted IGF1 was measured after 24 hours in the cell culture supernatant using a specific ELISA. Cpd.4 induced IGF1 secretion significantly more potent (EC₅₀ 0.134 μg) than Cpd.1 (EC₅₀ 0.889 μg). Data represent means±standard error of the mean of 2 replicates. Significance (***, <0.001) was assessed by two-way ANOVA of the two curves.

FIG. 19 shows the induction of IGF1 secretion from mouse skeletal muscle cells (C2C12) by mRNA transfection with Cpd.1-Cpd.7. C2C12 cells were transfected with each 2 μg Cp.1-Cpd.7, and secreted IGF1 was measured after 24 hours in the cell culture supernatant using a specific ELISA. Cpd.4 induced IGF1 secretion significantly higher than Cpd.1 (6.1-fold). Data represent means±standard error of the mean of 4 replicates. Significance (***, <0.001) was assessed by one-way ANOVA followed by Dunnett's multiple comparison test.

FIG. 20 shows the induction of IGF1 secretion from human primary skeletal muscle cells (HSkMC) by mRNA transfection with Cpd.1 and Cpd.4. HSkMC cells were transfected with each 2 μg Cp.1 or Cpd.4, and secreted IGF1 was measured after 24 hours in the cell culture supernatant using a specific ELISA. Cpd.4 induced IGF1 secretion significantly higher than Cpd.1 (3.1-fold). Data represent means±standard error of the mean of 3 replicates. Significance (**, p<0.01) was assessed by one-way ANOVA followed by Dunnett's multiple comparison test.

FIG. 21 shows functional recovery of tibialis anterior (TA) muscle after notexin injury. After notexin injury via intramuscular injection (day 0), two IGF-I mRNA treatments (Cpd. 4 (1 μg)) were applied by intramuscular injection on day 1 and 4 (see arrow heads). The control group received vehicle solution. Muscle function was assessed on day 1, 4, 7, 10, 14, 21 and 28 post-injury. Data represent means±standard error of the mean (SEM) of 5 mice per group and time point. Asterisks indicate significant difference of the Cpd. 4 treated group from the control group (p<0.05) as assessed by student's t-test.

FIG. 22 shows the induction of IGF1 secretion from human embryonic kidney cells (HEK293T) by mRNA transfection with Cpd.1 as control and Cpd.8-Cpd.26. HEK293T cells were transfected with each 0.3 μg Cpd.1 and Cpd.8-Cpd.26, and secreted IGF1 was measured after 24 hours in the cell culture supernatant using a specific ELISA. IGF1 secretion was normalized against Cpd.1. IGF1 secretion. Cpd. 8, 9, 10, 11, 12 and 13 showed a reduced IGF1 secretion whereas Cpd.14, 15, 16, 17, 18, 19, 20, 21, 23, 24, 25 and 26 induced IGF1 secretion higher than Cpd.1 (up to 2.6-fold). Data represent means±standard error of the mean of 2-11 replicates per Cpd. Significance (*, p<0.05; **, p<0.001; ***, <0.001) was assessed by Student's t-test of an individual Cpd. compared to Cpd.1.

FIG. 23 shows the induction of IGF1 secretion from human hepatic cells (HepG2) by mRNA transfection with Cpd.1 as control and Cpd.4-Cpd.26. HepG2 cells were transfected with each 0.3 μg Cp.1 and Cpd.4-Cpd.26, and secreted IGF1 was measured after 24 hours in the cell culture supernatant using a specific ELISA. IGF1 secretion was normalized against Cpd.1. Cpd. 8, 9 and 12 showed a reduced IGF1 secretion whereas Cpd. 4, 15, 16, 17, 18, 19, 20, 21, 22, 23, 24, 25 and 26 induced IGF1 secretion higher than Cpd.1 (up to 8.3-fold). Data represent means±standard error of the mean of 2-4 replicates per Cpd. Significance (**, p<0.01; ***, <0.001) was assessed by Student's t-test of an individual Cpd. compared to Cpd.1.

FIG. 24 shows the induction of IGF1 secretion from human neuronal cells (IMR32) by mRNA transfection with Cpd.1 as control and Cpd.4-Cpd.24. IMR32 cells were transfected with each 0.3 μg Cp.1 and Cpd.4-Cpd.24, and secreted IGF1 was measured after 24 hours in the cell culture supernatant using a specific ELISA. IGF1 secretion was normalized against Cpd.1. Cpd. 4, 14, 15, 16, 17, 20, 22, 23 and 24 induced IGF1 secretion higher than Cpd.1 (up to 2.6-fold). Data represent means±standard error of the mean of 2-6 replicates per Cpd. Significance (*, p<0.05; ***, <0.001) was assessed by Student's t-test of an individual Cpd. compared to Cpd.1.

FIG. 25 shows the induction of IGF1 secretion from human primary chondrocytes by mRNA transfection with Cpd.1 as control and Cpd.4-Cpd.25. Chondrocytes were transfected with each 0.6 μg Cp.1 and Cpd.4-Cpd.25, and secreted IGF1 was measured after 24 hours in the cell culture supernatant using a specific ELISA. IGF1 secretion was normalized against Cpd.1. Cpd. 4, 14, 15, 16, 20, 21, 22, 24 and 25 induced IGF1 secretion higher than Cpd.1 (up to 1.9-fold). Data represent means±standard error of the mean of 1-2 replicates per Cpd. Significance (*, p<0.05; ***, <0.001) was assessed by Student's t-test of an individual Cpd. compared to Cpd.1.

FIGS. 26A-26B show the induction of IGF1 secretion from rat wild type (FIG. 26A) or SOD1G^{S93A} (FIG. 26B) primary motoneurons by mRNA transfection with Cpd.1 as control and Cpd.4-Cpd.17. Rat wild type primary motoneurons were transfected with each 0.3 μg Cp.1, Cpd.4, Cpd.14 and Cpd.17 and rat SOD1G^{S93A} primary motoneurons were transfected with each 0.3 μg Cp.1, Cpd.14 and Cpd.17, and secreted IGF1 was measured after 48 hours in the cell culture supernatant using a specific ELISA. IGF1 secretion was normalized against Cpd.1. Cpd. 4, 14 and 17 induced IGF1 secretion higher than Cpd.1 (up to 4.3-fold in wild type or 9.3-fold in SOD1^{S93A}). Data represent means±standard error of the mean of 2 replicates per Cpd. Significance was assessed by Student's t-test of an individual Cpd. compared to Cpd.1 and revealed no statistical difference.

FIGS. 27A-27C show the induction of EPO secretion from human embryonic kidney cells (HEK293T, FIG. 27A), human hepatic cells (HepG2, FIG. 27B) and human lung carcinoma cells (A549, FIG. 27C) by mRNA transfection with Cpd.27, Cpd.28 or Cpd.29. Cells were transfected with each 0.3-0.9 μg Cp.27, Cpd.28 or Cpd.29, and secreted EPO was measured after 24 hours in the cell culture supernatant using a specific ELISA. EPO secretion was normalized against Cpd.27. Cpd. 28 and 29 induced EPO secretion higher than Cpd.27 in all three cell types analyzed (up to 1.8-fold). Data represent means±standard error of the mean of 3-8 replicates per Cpd. Significance (*, p<0.05; ***, <0.001) was assessed by Student's t-test of an individual Cpd. compared to Cpd.27.

FIG. 28 shows the induction of INS secretion from human embryonic kidney cells (HEK293T) by mRNA transfection with Cpd.30, Cpd.31 or Cpd.32. Cells were transfected with each 0.6 μg Cp.30, Cpd.31 or Cpd.32, and secreted INS was measured after 24 hours in the cell culture supernatant using a specific ELISA. INS secretion was normalized against Cpd.30. Cpd. 31 and 32 induced INS secretion higher than Cpd.30 (up to 3.9-fold). Data represent means±standard error of the mean of 3-5 replicates per Cpd. Significance (*, p<0.05; ***, <0.001) was assessed by Student's t-test of an individual Cpd. compared to Cpd.30.

FIGS. 29A-29D show the induction of IL4 secretion from human embryonic kidney cells (HEK293T, FIG. 29A), human hepatic cells (HepG2, FIG. 29B), human monocytes (THP-1, FIG. 29C) and human lung carcinoma cells (A549, FIG. 29D) by mRNA transfection with Cpd.33, Cpd.34 or Cpd.35. Cells were transfected with each 0.5-0.6 μg Cp.33, Cpd.34 or Cpd.35, and secreted IL4 was measured after 24 hours in the cell culture supernatant using a specific ELISA. IL4 secretion was normalized against Cpd.33. Cpd. 34 and 35 induced IL4 secretion higher than Cpd.33 in all three cell types analyzed (up to 2.2-fold). Data represent means±standard error of the mean of 3-8 replicates per Cpd. Significance (*, p<0.05; ***, <0.001) was assessed by Student's t-test of an individual Cpd. compared to Cpd.33.

FIGS. 30A-30C show the induction of IL10 secretion from human embryonic kidney cells (HEK293T, FIG. 30A), human hepatic cells (HepG2, FIG. 30B) or human monocytes (THP-1, FIG. 30C) by mRNA transfection with Cpd.36, Cpd.37 or Cpd.38. Cells were transfected with each 0.3-0.6 μg Cp.36, Cpd.37 or Cpd.38, and secreted IL10 was measured after 24 hours in the cell culture supernatant using a specific ELISA. IL10 secretion was normalized against Cpd.36. Cpd. 37 and 38 induced IL10 secretion higher than Cpd.36 in all three cell types analyzed (up to 2.2-fold). Data represent means±standard error of the mean of 4-8 replicates per Cpd. Significance (**, p<0.01; ***, <0.001) was assessed by Student's t-test of an individual Cpd. compared to Cpd.36.

FIGS. 31A-31B show the induction of IGF-1 secretion from human hepatic cells (HepG2, FIG. 31A) and human primary chondrocytes cells (FIG. 31B) by mRNA transfection with Cpd.39. Cells were transfected with each 0.3-0.6 μg with Cp.39 and secreted IGF1 was measured after 24 hours in the cell culture supernatant using a specific ELISA. IGF1 secretion was normalized against Cpd.1. Cpd. 39 induced IGF1 secretion higher than Cpd.1 in all two cell types analysed (up to 1.4-fold). Data represent means±standard error of the mean of 4-7 replicates. Significance (**, p<0.01; ***, <0.001) was assessed by Student's t-test of an individual Cpd.1 compared to Cpd.39.

### DETAILED DESCRIPTION OF THE INVENTION

The term “RNA” as used herein includes RNA which codes for an amino acid sequence as well as RNA which does not code for an amino acid sequence. Usually the RNA as used herein is a coding RNA, i.e. an RNA which codes for an amino acid sequence. Such RNA molecules are also referred to as mRNA (messenger RNA) and are single-stranded RNA molecules. Thus the term “RNA” as used herein preferably refers to mRNA. The RNA may be made by synthetic chemical and enzymatic methodology known to one of ordinary skill in the art, or by the use of recombinant technology, or may be isolated from natural sources, or by a combination thereof. The RNA may optionally comprise unnatural and naturally occurring nucleoside modifications such as e.g. N¹-Methylpseudouridine also referred herein as methylpseudouridine.

The term “mRNA” (i.e. messenger RNA) as used herein refers to polymers which are built up of nucleoside phosphate building blocks mainly with adenosine, cytidine, uridine and guanosine as nucleosides, and which contain a coding region encoding a protein. In the context of the present invention, mRNA should be understood to mean any polyribonucleotide molecule which, if it comes into the cell, is suitable for the expression of a protein or fragment thereof or is translatable to a protein or fragment thereof. The mRNA of the present invention comprising a nucleic acid sequence encoding a protein and a signal peptide should be understood to mean a polyribonucleotide molecule which, if it comes into the cell, is suitable to induce the expression and secretion of said protein or fragment thereof. The mRNA of the present invention is an artificial nucleic acid molecule, i.e. an artificial mRNA. An artificial nucleic acid molecule e.g. an artificial mRNA may typically be understood to be a nucleic acid molecule, that does not occur naturally, like a recombinant mRNA. A recombinant mRNA is the preferred mRNA of the present invention. The mRNA contains a ribonucleotide sequence which encodes a protein or fragment thereof whose function in the cell or in the vicinity of the cell is usually needed or beneficial, in particular in the context of the healing of skeletal muscle injuries. The mRNA may contain the sequence for the complete protein or a functional variant thereof. Thus the nucleic acid sequence of the mRNA for the complete protein usually comprises a nucleic acid sequence encoding the signal peptide and a nucleic acid sequence encoding the protein. The mRNA of the present invention comprises a nucleic acid sequence encoding a protein and a signal peptide. The nucleic acid sequence encoding a protein may optionally comprise the pro-domain of a protein, which is usually located at the N-terminus of the protein. The protein and the signal peptide are usually encoded by the nucleic acid sequence of the mRNA of the present invention in the following order from 5′ to 3′: i) the signal peptide and ii) the protein i.e the last nucleoside of the coding region of the signal peptide is followed by the first nucleoside of the coding region of the protein or in case of a protein comprising a pro-domain by the first nucleoside of the coding region of the pro-protein form of the protein. The ribonucleotide sequence can encode a protein which acts as a factor, inducer, regulator, stimulator or enzyme, or a functional fragment thereof, where this protein is usually one whose function is necessary in order to remedy a disorder, in particular a skeletal muscle injury. Here, functional variant is understood to mean a fragment which in the cell can undertake the function of the protein whose function in the cell is needed. In addition, the mRNA may also have further functional regions and/or 3′ or 5′ noncoding regions. The 3′ and/or 5′ noncoding regions can be the regions naturally flanking the protein-encoding sequence or artificial sequences which contribute to the stabilization of the RNA like e.g. a cap at the 5′ end and/or a polyA tail at the 3′ end. Those skilled in the art can determine the sequences suitable for this in each case by routine experiments. The mRNA or the DNA used to transcribe the mRNA may be codon optimized. Preferably, the DNA used in the present invention to transcribe the mRNA of the present invention and the mRNA of the present invention are codon optimized. In general, codon optimization refers to a process of modifying a nucleic acid sequence for expression in a host cell of interest by replacing at least one codon (e.g. more than 1, 2, 3, 4, 5, 10, 15, 20, 25, 50, or more codons) of a native sequence with codons that are more frequently or most frequently used in the genes of that host cell while maintaining the native amino acid sequence. Codon usage tables are readily available, for example, at the “Codon Usage Database”, and these tables can be adapted in a number of ways. Computer algorithms for codon optimizing a particular sequence for expression in a particular host cell are also available, such as Gene Forge® (Aptagen, PA) and GeneOptimizer® (ThermoFischer, MA) which is preferred.

The term “naked RNA” as used herein refers to a RNA which is not complexed to any kind of other compound, in particular proteins, peptides, polymers, like cationic polymers, lipids, liposomes, viral vectors or the like. Thus, “naked RNA” means that the RNA is present e.g. in a liquid composition in a free and uncomplexed form being molecularly dispersed in solution. For example, it is not envisaged that the “naked RNA” is complexed with a lipid and/or polymer carrier system (e.g., lipid nano particles and micelle)/transfection reagent like, for example, DreamFect™ Gold or (branched) PEI. Hence, a composition comprising the mRNA, like the therapeutic composition of the invention, does, for example, not contain a lipid and/or polymer carrier system transfection reagent like, for example, DreamFect™ Gold or (branched) PEI.

The terms “nucleic acid sequence”, “nucleotide sequence” and “nucleotide acid sequence” are used herein interchangeably and have the identical meaning herein, and refer to preferably DNA or RNA. The terms “nucleic acid sequence”, “nucleotide sequence” and “nucleotide acid sequence” are preferably used synonymous with the term “polynucleotide sequence”.

Preferably, a nucleic acid sequence is a polymer comprising or consisting of nucleotide monomers, which are covalently linked to each other by phosphodiester-bonds of a sugar/phosphate-backbone. The term “nucleic acid sequence” also encompasses modified nucleic acid sequences, such as base-modified, sugar-modified or backbone-modified etc. DNA or RNA.

The term “open reading frame” as used herein refers to a sequence of several nucleotide triplets, which may be translated into a peptide or protein. An open reading frame (ORF) preferably contains a start codon, i.e. a combination of three subsequent nucleotides coding usually for the amino acid methionine (ATG), at its 5′-end and a subsequent region, which usually exhibits a length which is a multiple of 3 nucleotides. An ORF is preferably terminated by a stop-codon (e.g., TAA, TAG, TGA). Typically, this is the only stop-codon of the open reading frame. Thus, an open reading frame in the context of the present invention is preferably a nucleic acid sequence, consisting of a number of nucleotides that may be divided by three, which starts with a start codon (e.g. ATG) and which preferably terminates with a stop codon (e.g., TAA, TGA, or TAG). The open reading frame may be isolated or it may be incorporated in a longer nucleic acid sequence, for example in a vector or an mRNA. An open reading frame may also be termed “(protein) coding region” or, preferably, “coding sequence”.

The term “signal peptide” also referred herein to as signalling peptide, pre-domain, signal sequence, targeting signal, localization signal, localization sequence, transit peptide, leader sequence or leader peptide is a short peptide (usually 16-40 amino acids long) present at the N-terminus of newly synthesized proteins that are destined towards the secretory pathway. The signal peptide of the present invention is preferably 10-50, more preferably 11-45, even more preferably 12-45, most preferably 13-45, in particular 14-45, more particular 15-45, even more particular 16-40 amino acids long. A signal peptide according to the invention is situated at the N-terminal end of the protein of interest or at the N-terminal end of the pro-protein form of the protein of interest. Using a signal peptide according to the invention, the protein of interest can be secreted in a quantity at least equal to, preferably in a quantity higher than the quantity of said protein secreted using its natural (homologous) signal peptide. A signal peptide according to the invention is usually of eukaryotic origin e.g. the signal peptide of a eukaryotic protein, preferably of mammalian origin e.g. the signal peptide of a mammalian protein, more preferably of human origin e.g. the signal peptide of a mammalian protein. In some embodiments the heterologous signal peptide and/or the homologous signal peptide to be modified is the naturally occurring signal peptide of a eukaryotic protein, preferably the naturally occurring signal peptide of a mammalian protein, more preferably the naturally occurring signal peptide of a human protein.

The term “protein” as used herein refers to molecules typically comprising one or more peptides or polypeptides. A peptide or polypeptide is typically a chain of amino acid residues, linked by peptide bonds. A peptide usually comprises between 2 and 50 amino acid residues. A polypeptide usually comprises more than 50 amino acid residues. A protein is typically folded into 3-dimensional form, which may be required for the protein to exert its biological function. The term “protein” as used herein includes a fragment of a protein and fusion proteins. Preferably the protein is of mammalian, more preferably human origin i.e. is a human protein. Preferably the protein is a protein which is normally secreted from a cell, i.e. a protein which is secreted from a cell in nature. Proteins as referred herein are usually selected from the group consisting of carboxypeptidases; cytokines; extracellular ligands and transporters; extracellular matrix proteins; glucosidases; glycosyltransferases; growth factors; growth factor binding proteins; heparin binding proteins; hormones; hydrolases; immunoglobulins; isomerases; kinases; lyases; metalloenzyme inhibitors; metalloproteases; milk proteins; neuroactive proteins; proteases; protease inhibitors; protein phosphatases; esterases; transferases; and vasoactive proteins.

Carboxypeptidases are proteins which are protease enzymes that hydrolyze (cleave) a peptide bond at the carboxy-terminal (C-terminal) end of a protein; cytokines are proteins which are secreted and act either locally or systemically as modulators of target cell signalling via receptors on their surfaces, often involved in immunologic reactions; extracellular ligands and transporters are proteins that are secreted and act via binding to other proteins or carrying other proteins or other molecules to exert a certain biological function; extracellular matrix proteins are a collection of proteins secreted by support cells that provide structural and biochemical support to the surrounding cells; glucosidases are enzymes involved in breaking down complex carbohydrates such as starch and glycogen into their monomers; glycosyltransferases are enzymes that establish natural glycosidic linkages, growth factors are secreted proteins capable of stimulating cellular growth, proliferation, healing, and cellular differentiation either acting locally or systemically as modulators of target cell signalling via receptors on their surfaces, often involved in trophic reactions and survival or cell homeostasis signalling; growth factor binding proteins are secreted proteins binding to growth factors and thereby modulating their biological activity; heparin binding proteins are secreted proteins that interact with heparin to modulate their biological function, often in conjunction with another binding to a growth factor or hormone; hormones are members of a class of signaling molecules produced by glands in multicellular organisms that are secreted and transported by the circulatory system to target distant organs to regulate physiology and behaviour via binding to specific receptors on their target cells; hydrolases are a class of enzymes that biochemically catalyze molecule cleavage by utilizing water to break chemical bonds, resulting in a division of a larger molecule to smaller molecules; immunoglobulins are large, Y-shaped secreted proteins produced mainly by plasma cells that are used by the immune system to neutralize pathogens such as pathogenic bacteria and viruses; isomerases are a general class of enzymes that convert a molecule from one isomer to another, thereby facilitating intramolecular rearrangements in which bonds are broken and formed; kinases are enzymes catalyzing the transfer of phosphate groups from high-energy, phosphate-donating molecules to specific substrates; lyases are enzymes catalyzing the breaking of various chemical bonds by means other than hydrolysis and oxidation, often forming a new double bond or a new ring structure; metalloenzyme inhibitors cellular inhibitors of the Matrix metalloproteases (MMPs); metalloproteases are protease enzymes whose catalytic mechanism involves a metal ion; milk proteins are proteins secreted into milk; neuroactive proteins are secreted proteins that act either locally or via distances to support neuronal function, survival and physiology; proteases (also called peptidases or proteinases) are enzymes that perform proteolysis by hydrolysis of peptide bonds; protease inhibitors are proteins that inhibit the function of proteases; protein phosphatases are enzymes that remove phosphate groups from phosphorylated amino acid residues of their substrate protein; esterases are enzymes that split esters into an acid and an alcohol in a chemical reaction with water at a aminoacid residue; transferases are a class of enzymes that catalyse the transfer of specific functional groups (e.g. a methyl or glycosyl group) from one molecule (called the donor) to another (called the acceptor); vasoactive proteins are secreted proteins that biologically affect function of blood vessels. Carboxypeptidases; cytokines; extracellular ligands and transporters; extracellular matrix proteins; glucosidases; glycosyltransferases; growth factors; growth factor binding proteins; heparin binding proteins; hormones; hydrolases; immunoglobulins; isomerases; kinases; lyases; metalloenzyme inhibitors; metalloproteases; milk proteins; neuroactive proteins; proteases; protease inhibitors; protein phosphatases; esterases; transferases; and vasoactive proteins as referred herein can be found in the UniProt database.

The term “fragment” or “fragment of a sequence” which have the identical meaning herein is a shorter portion of a full-length sequence of e.g. a nucleic acid molecule like DNA or RNA or a protein. Accordingly, a fragment, typically, comprises or consists of a sequence that is identical to the corresponding stretch within the full-length sequence. A preferred fragment of a sequence in the context of the present invention, comprises or consists of a continuous stretch of entities, such as nucleotides or amino acids corresponding to a continuous stretch of entities in the molecule the fragment is derived from, which represents at least 5%, usually at least 20%, preferably at least 30%, more preferably at least 40%, more preferably at least 50%, even more preferably at least 60%, even more preferably at least 70%, and most preferably at least 80% of the total (i.e. full-length) molecule, from which the fragment is derived.

The term “signal peptide heterologous to said protein” as used herein refers to a naturally occurring signal peptide which is different to the naturally occurring signal peptide of the protein, i.e. the signal peptide is not derived from the same gene of the protein. Usually a signal peptide heterologous to a given protein is a signal peptide from another protein, which is not related to the given protein i.e. which has an amino acid sequence which differs from the signal peptide of the given protein, e.g. which has an amino acid sequence which differs from the signal peptide of the given protein by more than 50%, preferably by more than 60%, more preferably by more than 70%, even more preferably by more than 80%, most preferably by more than 90%, in particular by more than 95%. Preferably a signal peptide heterologous to a given protein has a sequence identity with the amino acid sequence of the naturally occurring (homologous) signal peptide of the given protein of less than 95%, preferably less than 90%, more preferably less than 80%, even more preferably less than 70%, most preferably less than 60%, in particular less than 50%. Although heterologous sequences may be derivable from the same organism, they naturally (in nature) do not occur in the same nucleic acid molecule, such as in the same mRNA. The signal peptide heterologous to a protein and the protein to which the signal peptide is heterologous can be of the same or different origin and are usually of the same origin, preferably of eukaryotic origin, more preferably of eukaryotic origin of the same eukaryotic organism, even more preferably of mammalian origin, in particular of mammalian origin of the same mammalian organism, more particular of human origin. In Example 1 a mRNA comprising a nucleic acid sequence encoding the human BDNF signal peptide and the human IGF1, i.e. a signal peptide heterologous to a protein wherein the signal peptide and the protein are of the same origin, namely of human origin is disclosed.

The term “signal peptide homologous to said protein” as used herein refers to the naturally occurring signal peptide of a protein. A signal peptide homologous to a protein is the signal peptide encoded by the gene of the protein as it occurs in nature. A signal peptide homologous to a protein is usually of eukaryotic origin e.g. the naturally occurring signal peptide of a eukaryotic protein, preferably of mammalian origin e.g. the naturally occurring signal peptide of a mammalian protein, more preferably of human origin e.g. the naturally occurring signal peptide of a human protein.

The term “naturally occurring amino acid sequence which does not have the function of a signal peptide in nature” as used herein refers to an amino acid sequence which occurs in nature and which is not identical to the amino acid sequence of any signal peptide occurring in nature. The naturally occurring amino acid sequence which does not have the function of a signal peptide in nature as referred to in the present invention is preferably between 10-50, more preferably 11-45, even more preferably 12-45, most preferably 13-45, in particular 14-45, more particular 15-45, even more particular 16-40 amino acids long. Preferably the naturally occurring amino acid sequence which does not have the function of a signal peptide in nature of the present invention is of eukaryotic origin and not identical to any signal peptide of eukaryotic origin, more preferably is of mammalian origin and not identical to any signal peptide of mammalian origin, more preferably is of human origin and not identical to any signal peptide of human origin occurring in nature. A naturally occurring amino acid sequence which does not have the function of a signal peptide in nature is usually an amino acid sequence of the coding sequence of a protein. A naturally occurring amino acid sequence which does not have the function of a signal peptide in nature according to the present invention is usually of eukaryotic origin, preferably of mammalian origin, more preferably of human origin.

The term “naturally occurring”, “natural” and “in nature” as used herein have the equivalent meaning.

The term “amino acids 1-9 of the N-terminal end of the amino acid sequence of the signal peptide” as used herein refers to the first nine amino acids of the N-terminal end of the amino acid sequence of a signal peptide. Analogously the term “amino acids 1-7 of the N-terminal end of the amino acid sequence of the signal peptide” as used herein refers to the first seven amino acids of the N-terminal end of the amino acid sequence of a signal peptide and the term” “amino acids 1-5 of the N-terminal end of the amino acid sequence of the signal peptide” as used herein refers to the first five amino acids of the N-terminal end of the amino acid sequence of a signal peptide.

The term “amino acid sequence modified by insertion, deletion and/or substitution of at least one amino acid” as used herein refers to an amino acid sequence which includes an amino acid substitution, insertion, and/or deletion of at least one amino acid within the amino acid sequence. The term “signal peptide heterologous to said protein is modified by insertion, deletion and/or substitution of at least one amino acid” as used herein refers to an amino acid sequence of a naturally occurring signal peptide heterologous to a protein which includes an amino acid substitution, insertion, and/or deletion of at least one amino acid within its naturally occurring amino acid sequence. The term “signal peptide homologous to said protein is modified by insertion, deletion and/or substitution of at least one amino acid” as used herein refers to a natural occurring signal peptide homologous to a protein which includes an amino acid substitution, insertion, and/or deletion of at least one amino acid within its naturally occurring amino acid sequence. The term “the naturally occurring amino acid sequence is modified by insertion, deletion and/or substitution of at least one amino acid” refers to a naturally occurring amino acid sequence which includes an amino acid substitution, insertion, and/or deletion of at least one amino acid within its naturally occurring amino acid sequence. By “amino acid substitution” or “substitution” herein is meant the replacement of an amino acid at a particular position in a parent protein sequence with another amino acid. For example, the substitution R34K refers to a polypeptide, in which the arginine at position 34 is replaced with a lysine. For the preceding example, 34K indicates the substitution of position 34 with a lysine. For the purposes herein, multiple substitutions are typically separated by a slash. For example, R34K/L78V refers to a double variant comprising the substitutions R34K and L38V. By “amino acid insertion” or “insertion” as used herein is meant the addition of an amino acid at a particular position in a parent protein sequence. For example, insert −34 designates an insertion at position 34. By “amino acid deletion” or “deletion” as used herein is meant the removal of an amino acid at a particular position in a parent protein sequence. For example, R34- designates the deletion of arginine at position 34.

Preferably the deleted amino acid is an amino acid with a hydrophobic score of below −0.8, preferably below 1.9. Preferably the substitute amino acid is an amino acid with a hydrophobic score which is higher than the hydrophobic score of the substituted amino acid, more preferably the substitute amino acid is an amino acid with a hydrophobic score of 2.8 and higher, more preferably with a hydrophobic score of 3.8 and higher. Preferably the inserted amino acid is an amino acid with a hydrophobic score of 2.8 and higher, more preferably with a hydrophobic score of 3.8 and higher.

Usually between 1 and 15, preferably between 1 and 11 amino acids, more preferably between 1 and 10 amino acids, even more preferably between 1 and 9 amino acids, in particular between 1 and 8 amino acids, more particular between 1 and 7 amino acids, even more particular between 1 and 6 amino acids, particular preferably between 1 and 5 amino acids, more particular preferably between 1 and 4 amino acids, even more particular preferably between 1 and 2 amino acids of a given amino acid sequence are inserted, deleted and/or substituted. Usually between 1 and 15, preferably between 1 and 11 amino acids, more preferably between 1 and 10 amino acids, even more preferably between 1 and 9 amino acids, in particular between 1 and 8 amino acids, more particular between 1 and 7 amino acids, even more particular between 1 and 6 amino acids, particular preferably between 1 and 5 amino acids, more particular preferably between 1 and 4 amino acids, even more particular preferably between 1 and 2 amino acids of a given amino acid sequence are inserted, deleted and/or substituted usually within the amino acids 1-11, preferably within the amino acids 1-10, more preferably within the amino acids 1-9, even more preferably within the amino acids 1-8, in particular within the amino acids 1-7, more particular within the amino acids 1-6, even more particular within the amino acids 1-5, particular preferably within the amino acids 1-4, more particular preferably within the amino acids 1-3, even more particular preferably within the amino acids 1-2 of the N-terminal end of the amino acid sequence of the signal peptide. Preferably the amino acid sequence is optionally modified by deletion and/or substitution of at least one amino acid.

Preferably, the average hydrophobic score of the first nine amino acids of the N-terminal end of the amino acid sequence of the modified signal peptide is increased 1.0 unit or above compared to the signal peptide without modification.

The term “insulin-like growth factor 1”, “insulin-like growth factor 1 (IGF1)” or “IGF1” as used herein usually refers to the natural sequence of the IGF1 protein without the signalling peptide and may comprise the propeptide and/or the E-peptide and preferably refers to the natural sequence of the IGF1 protein without the signalling peptide and without the E-peptide.

The term “human insulin-like growth factor 1 (IGF1)” as used herein refers to the natural sequence of human IGF1 (pro-IGF1 which is referred to in the Uniprot database as UniProtKB-P05019 and in the Genbank database as NM_000618.4, NM_001111285.2 and NM_001111283.2, or a fragment thereof. The natural DNA sequence encoding human insulin-like growth factor 1 may be codon-optimized. The natural sequence of human IGF1 comprises or consists of the human signalling peptide having 21 amino acids (nucleotides 1-63), the human propeptide (also called pro-domain) having 27 amino acids (nucleotides 64-144), the mature human IGF1 having 70 amino acids (nucleotides 145-354) and the C-terminal domain of human IGF1 which is the so-called E-peptide (or E-domain). The C-terminal domain of human IGF1 (so called E-peptide or E-domain) comprises the Ea-, Eb- or Ec-domain which are generated by alternative splicing events. The Ea-domain comprises or consists of 35 amino acids (105 nucleotides), the Eb-domain comprises or consists of 77 amino acids (231 nucleotides), and the Ec-domain comprises or consists of 40 amino acids (120 nucleotides) (see e.g. Wallis M (2009) New insulin-like growth factor (IGF)-precursor sequences from mammalian genomes: the molecular evolution of IGFs and associated peptides in primates. Growth Horm IGF Res 19(1):12-23. doi: 10.1016/j.ghir.2008.05.001).

The term “human insulin-like growth factor 1 (IGF)” as used herein usually refers to the natural sequence of the human IGF1 protein without the signalling peptide and may comprise the propeptide and/or the E-peptide and preferably refers to the natural sequence of the human IGF1 protein without the signalling peptide and without the E-peptide. The term “human insulin-like growth factor 1 (IGF)” as used herein usually comprises the mature human IGF1.

The term “mature protein” refers to the protein synthesised in the endoplasmatic reticulum and secreted via the Golgi apparatus in a cell expressing and secreting the protein. The term “mature IGF1” refers to the protein synthesised in the endoplasmatic reticulum and secreted via the Golgi apparatus in a cell expressing and secreting IGF1. The term “mature human IGFI” refers to the protein synthesised in the endoplasmatic reticulum and secreted via the Golgi apparatus in a human cell expressing and secreting human IGF1 and normally contains the amino acids encoded by nucleotide sequence as shown in SEQ ID NO:39.

The term “insulin” or “INS” as used herein usually refers to the natural sequence of insulin without the signalling peptide. The term “human insulin” or “human INS” as used herein refers to the natural sequence of human insulin which is referred to in the Uniprot database as UniProtKB-P01308 and in the Genbank database as NM_000207.2, NM_001185097.1, NM_001185098.1 and NM_001291897.1, or a fragment thereof. The natural DNA sequence encoding human insulin may be codon-optimized. The natural sequence of human insulin comprises or consists of the human signalling peptide having 24 amino acids (nucleotides 1-72), the human insulin B-chain having 30 amino acids (nucleotides 73-163), the human insulin propeptide (also called connecting peptide; C-peptide) having 31 amino acids (nucleotides 64-144), and the C-terminal domain of human insulin A-chain comprises or consists of 21 amino acids (nucleotides 64-144). The term “human insulin” as used herein usually comprises the human insulin without the signalling peptide.

The term “Erythropoietin”, “EPO” or “Epo” as used herein usually refers to the natural sequence of EPO without the signalling peptide. The term “human Erythropoietin”, “human EPO” or “human Epo” as used herein refer to the natural sequence of human erythropoietin which is referred to in the Uniprot database as UniProtKB-P01588 and in the Genbank database as NM_000799.2, or a fragment thereof. The natural DNA sequence encoding human erythropoietin may be codon-optimized. The natural sequence of human erythropoietin comprises or consists of the human signalling peptide having 27 amino acids (nucleotides 1-81), the human Epo coding chain having 166 amino acids (nucleotides 82-579). The term “human erythropoietin” as used herein usually comprises the human EPO without the signalling peptide.

The term “Interleukin-4” or “IL4” as used herein usually refers to the natural sequence of IL4 without the signalling peptide. The term “human Interleukin-4” or “human IL4” as used herein refer to the natural sequence of human IL4 which is referred to in the Uniprot database as UniProtKB-P05112 and in the Genbank database as NM_000589.3 and NM_172348.2 or a fragment thereof. The natural DNA sequence encoding human IL4 may be codon-optimized. The natural sequence of human IL4 comprises or consists of the human signalling peptide having 24 amino acids (nucleotides 1-72), the human IL4 coding chain having 129 amino acids (nucleotides 73-387). The term “human IL4” as used herein usually comprises the human IL4 without the signalling peptide.

The term “Interleukin-10” or “IL10” as used usually herein refer to the natural sequence of IL10 without the signalling peptide. The term “human Interleukin-10” or “human IL10” as used herein refer to the natural sequence of human IL10 which is referred to in the Uniprot database as UniProtKB-P22301 and in the Genbank database as NM_000572.2 or a fragment thereof. The natural DNA sequence encoding human IL10 may be codon-optimized. The natural sequence of human IL10 comprises or consists of the human signalling peptide having 18 amino acids (nucleotides 1-54), the human IL10 coding chain having 160 amino acids (nucleotides 55-534). The term “human IL10” as used herein usually comprises the human IL10 without the signalling peptide.

The term “signal peptide of the Insulin growth factor 1 (IGF1)” or “signal peptide of IGF1” as used herein refers to the natural signal peptide of IGF1 which is referred to in the Uniprot database as P05019 and in the Genbank database as NM_000618.4, NM_001111284.1 and NM_001111285.2 and has preferably the amino acid sequence as shown in SEQ ID NO: 24 and/or is preferably encoded by the DNA sequence as shown in SEQ ID NO: 25.

The term “signal peptide of the Insulin growth factor 2 (IGF2)” or “signal peptide of IGF2” as used herein refers to the natural signal peptide of IGF2 which is referred to in the Uniprot database as P01344 and in the Genbank database as NM_000612.5, NM_001007139.5, NM_001127598.2, NM_001291861.2 and NM_001291862.2 and has preferably the amino acid sequence as shown in SEQ ID NO: 26 and/or is preferably encoded by the DNA sequence as shown in SEQ ID NO: 27.

The term “signal peptide of the serum albumin (ALB)” or “signal peptide of ALB” as used herein refers to the natural signal peptide of ALB which is referred to in the Uniprot database as P02768 and in the Genbank database as NM_000477.6 and has preferably the amino acid sequence as shown in SEQ ID NO: 28 and/or is preferably encoded by the DNA sequence as shown in SEQ ID NO: 29.

The term “brain-derived neurotrophic factor (BDNF)” or “signal peptide of BDNF” as used herein refers to the natural signal peptide of BDNF which is referred to in the Uniprot database as P23560 and in the Genbank database as NM_001143805.1, NM_170731.4, NM_170734.3, NM_001143810.1 and NM_001143809.1 and has preferably the amino acid sequence as shown in SEQ ID NO: 30 and/or is preferably encoded by the DNA sequence as shown in SEQ ID NO: 31.

The term “signal peptide of stromal cell derived factor-1 (CXCL12)” or “signal peptide of CXCL12” as used herein refers to the natural signal peptide of CXCL12 which is referred to in the Uniprot database as P48061 and in the Genbank database as NM_000609.6, NM_001033886.2, NM_001178134.1, NM_001277990.1 and NM_199168.3 and has preferably the amino acid sequence as shown in SEQ ID NO:32 and/or is preferably encoded by the DNA sequence as shown in SEQ ID NO: 33.

The term “signal peptide of synthetic signalling peptide 1 (synthetic seq1)” or “signal peptide of synthetic seq1” as used herein refers to the synthetic signal peptide 1 and has the amino acid sequence as shown in SEQ ID NO: 34 and/or is encoded by the DNA sequence as shown in SEQ ID NO: 35.

The term “signal peptide of synthetic signalling peptide 2 (synthetic seq2)” or “signal peptide of synthetic seq1” as used herein refers to the synthetic signal peptide 1 and has the amino acid sequence as shown in SEQ ID NO: 36 and/or is encoded by the DNA sequence as shown in SEQ ID NO: 37.

The term “signal peptide of the Latent-transforming growth factor beta-binding protein 2 (LTBP2)” or “signal peptide of LTBP2” as used herein refers to the natural signal peptide of LTBP2 which is referred to in the Uniprot database as Q14767 and in the Genbank database as NM_000428.2 and has preferably the amino acid sequence as shown in SEQ ID NO: 41 and/or is preferably encoded by the DNA sequence as shown in SEQ ID NO: 42.

The term “signal peptide of the Insulin-like growth factor-binding protein complex acid labile subunit (IGFALS)” or “signal peptide of IGFALS” as used herein refers to the natural signal peptide of IGFALS which is referred to in the Uniprot database as P35858 and in the Genbank database as NM_001146006.1 and NM_004970.2 and has preferably the amino acid sequence as shown in SEQ ID NO: 46 and/or is preferably encoded by the DNA sequence as shown in SEQ ID NO: 47.

The term “signal peptide of the Insulin (INS)” or “signal peptide of INS” as used herein refers to the natural signal peptide of INS which is referred to in the Uniprot database as P1308 and in the Genbank database as NM_001185097.1, NM_000207.2, NM_001185098.1 and NM_001291897.1 and has preferably the amino acid sequence as shown in SEQ ID NO: 51 and/or is preferably encoded by the DNA sequence as shown in SEQ ID NO: 52.

The term “signal peptide of the Erythropoietin (Epo)” or “signal peptide of Epo” as used herein refers to the natural signal peptide of Epo which is referred to in the Uniprot database as P01588 and in the Genbank database as NM_000799.2 and has preferably the amino acid sequence as shown in SEQ ID NO: 56 and/or is preferably encoded by the DNA sequence as shown in SEQ ID NO: 57.

The term “signal peptide of the Granulocyte colony-stimulating factor (CSF3)” or “signal peptide of CSF3” as used herein refers to the natural signal peptide of CSF3 which is referred to in the Uniprot database as P09919 and in the Genbank database as NM_000759.3, NM_001178147.1, NM_172219.2 and NM_172220.2 and has preferably the amino acid sequence as shown in SEQ ID NO: 61 and/or is preferably encoded by the DNA sequence as shown in SEQ ID NO: 62.

The term “signal peptide of the Beta-nerve growth factor (NGF)” or “signal peptide of NGF” as used herein refers to the natural signal peptide of NGF which is referred to in the Uniprot database as P01138 and in the Genbank database as NM_002506.2 and XM_006710663.3 and has preferably the amino acid sequence as shown in SEQ ID NO: 66 and/or is preferably encoded by the DNA sequence as shown in SEQ ID NO: 67.

The term “signal peptide of the Interleukin-4 (IL4)” or “signal peptide of IL4” as used herein refers to the natural signal peptide of IL4 which is referred to in the Uniprot database as P05112 and in the Genbank database as NM_000589.3 and NM_172348.2 and has preferably the amino acid sequence as shown in SEQ ID NO: 77 and/or is preferably encoded by the DNA sequence as shown in SEQ ID NO: 78.

The term “signal peptide of the Interleukin-10 (IL10)” or “signal peptide of IL10” as used herein refers to the natural signal peptide of IL10 which is referred to in the Uniprot database as P22301 and in the Genbank database as NM_000572.2 and has preferably the amino acid sequence as shown in SEQ ID NO: 82 and/or is preferably encoded by the DNA sequence as shown in SEQ ID NO: 83.

The term “signal peptide of the Fibroblast growth factor 5 (FGF5)” or “signal peptide of FGF5” as used herein refers to the natural signal peptide of FGF5 which is referred to in the Uniprot database as P12034 and in the Genbank database as NM_004464.3 and NM_033143.2 and has preferably the amino acid sequence as shown in SEQ ID NO: 87 and/or is preferably encoded by the DNA sequence as shown in SEQ ID NO: 88 or SEQ ID NO: 183.

The term “signal peptide of the Complement factor H-related protein 2 (FHR2)” or “signal peptide of FHR2” as used herein refers to the natural signal peptide of FHR2 which is referred to in the Uniprot database as P36980 and in the Genbank database as NM_001312672.1 and NM_005666.3 and has preferably the amino acid sequence as shown in SEQ ID NO: 92 and/or is preferably encoded by the DNA sequence as shown in SEQ ID NO: 93.

The term “signal peptide of the Insulin-like growth factor-binding protein 5 (IBP5)” or “signal peptide of IBP5” as used herein refers to the natural signal peptide of IBP5 which is referred to in the Uniprot database as P24593 and in the Genbank database as NM_001312672.1 and NM_000599.3 and has preferably the amino acid sequence as shown in SEQ ID NO: 97 and/or is preferably encoded by the DNA sequence as shown in SEQ ID NO: 98.

The term “signal peptide of the Neurotrophin-3 (NTF3)” or “signal peptide of NTF3” as used herein refers to the natural signal peptide of NTF3 which is referred to in the Uniprot database as P20783 and in the Genbank database as NM_002527.4, XM_011520963.2 and NM_001102654.1 and has preferably the amino acid sequence as shown in SEQ ID NO: 102 and/or is preferably encoded by the DNA sequence as shown in SEQ ID NO: 103.

The term “signal peptide of the Prostate and testis expressed protein 2 (PATE2)” or “signal peptide of PATE2” as used herein refers to the natural signal peptide of PATE2 which is referred to in the Uniprot database as Q6UY27 and in the Genbank database as NM_212555.2 and has preferably the amino acid sequence as shown in SEQ ID NO: 107 and/or is preferably encoded by the DNA sequence as shown in SEQ ID NO: 108.

The term “signal peptide of the Extracellular superoxide dismutase (SOD3)” or “signal peptide of SOD3” as used herein refers to the natural signal peptide of SOD3 which is referred to in the Uniprot database as P08294 and in the Genbank database as NM_003102.2 and has preferably the amino acid sequence as shown in SEQ ID NO: 112 and/or is preferably encoded by the DNA sequence as shown in SEQ ID NO: 113.

The term “coding sequence of the Glucagon receptor (GL-R)” or “coding sequence of GL-R” as used herein refers to the coding chain of GL-R which is a naturally occurring amino acid sequence which does not have the function of a signal peptide in nature and is referred to in the Uniprot database as P47871 and in the Genbank database as NM_000160.4 and XM_006722277.1 and has preferably the amino acid sequence as shown in SEQ ID NO: 117 and/or is preferably encoded by the DNA sequence as shown in SEQ ID NO: 118.

The term “signal peptide of the Insulin growth factor 1 (IGF1) Modified”, “modified signal peptide of IGF1” or “signal peptide of IGF1-Modified” as used herein refers to the modified signal peptide of IGF1 wherein natural signal peptide of IGF1 which is referred to in the Uniprot database as P05019 and in the Genbank database as NM_000618.4, NM_001111284.1 and NM_001111285.2 is modified by the substitutions G2L/S5L/T9L/Q10L and deletions K3- and C15- and has preferably the amino acid sequence as shown in SEQ ID NO: 122 and/or is preferably encoded by the DNA sequence as shown in SEQ ID NO: 123.

The term “signal peptide of the Insulin growth factor 2 (IGF2) Modified”, “modified signal peptide of IGF2” or “signal peptide of IGF2-Modified” as used herein refers to the modified signal peptide of IGF2 wherein natural signal peptide of IGF2 is referred to in the Uniprot database as P01344 and in the Genbank database as NM_000612.5, NM_001007139.5, NM_001127598.2, NM_001291861.2 and NM_001291862.2 is modified by the substitutions G2L/G6L/K7L/S8L and deletions P4-, M5-, I23- and A24- and has preferably the amino acid sequence as shown in SEQ ID NO: 127 and/or is preferably encoded by the DNA sequence as shown in SEQ ID NO: 128.

The term “signal peptide of stromal cell derived factor-1 (CXCL12) Modified”, “modified signal peptide of CXCL12” or “signal peptide of CXCL12-Modified” as used herein refers to the modified signal peptide of CXCL12 wherein natural signal peptide of CXCL12 is referred to in the Uniprot database as P48061 and in the Genbank database as NM_000609.6, NM_001033886.2, NM_001178134.1, NM_001277990.1 and NM_199168.3 is modified by the deletions N3- and K5- and has preferably the amino acid sequence as shown in SEQ ID NO: 132 and/or is preferably encoded by the DNA sequence as shown in in SEQ ID NO: 133.

The term “signal peptide of the Interleukin-4 (IL4) Modified”, “modified signal peptide of IL4” or “signal peptide of IL4-Modified” as used herein refers to the modified signal peptide of IL4 wherein natural signal peptide of IL4 is referred to in the Uniprot database as P05112 and in the Genbank database as NM_000589.3 and NM_172348.2 is modified by the deletions G2-, T4-, S5- and Q6- and has preferably the amino acid sequence as shown in SEQ ID NO: 166 and/or is preferably encoded by the DNA sequence as shown in SEQ ID NO: 167.

The term “signal peptide of the Interleukin-10 (IL10) Modified”, “modified signal peptide of IL10” or “signal peptide of IL10-Modified” as used herein refers to the modified signal peptide of IL10 wherein natural signal peptide of IL10 is referred to in the Uniprot database as P22301 and in the Genbank database as NM_000572.2 is modified by the substitutions H2V/S3L/S4L and S8L and has preferably the amino acid sequence as shown in SEQ ID NO: 174 and/or is preferably encoded by the DNA sequence as shown in in SEQ ID NO: 175.

The term “signal peptide of the Insulin (INS) Modified”, “modified signal peptide of INS” or “signal peptide of INS-Modified” as used herein refers to the modified signal peptide of INS wherein natural signal peptide of INS is referred to in the Uniprot database as P1308 and in the Genbank database as NM_001185097.1, NM_000207.2, NM_001185098.1 and NM_001291897.1 is modified by the deletions M5- and R6- and has preferably the amino acid sequence as shown in SEQ ID NO: 147 and/or is preferably encoded by the DNA sequence as shown in SEQ ID NO: 148 or SEQ ID NO: 182.

The term “signal peptide of the brain-derived neurotrophic factor (BDNF) Modified”, “modified signal peptide of BDNF” or “signal peptide of BDNF-Modified” as used herein refers to the modified signal peptide of BDNF wherein natural signal peptide of BDNF is referred to in the Uniprot database as P23560 and in the Genbank database as NM_001143805.1, NM_170731.4, NM_170734.3, NM_001143810.1 and NM_001143809.1 is modified by the substitutions T2L/T7L and S11L and has preferably the amino acid sequence as shown in SEQ ID NO: 137 and/or is preferably encoded by the DNA sequence as shown in in SEQ ID NO: 138.

The term “signal peptide of the Erythropoietin (Epo) Modified”, “modified signal peptide of Epo” or “signal peptide of Epo-Modified” as used herein refers to the modified signal peptide of Epo wherein natural signal peptide of Epo is referred to in the Uniprot database as P01588 and in the Genbank database as NM_000799.2 is modified by the substitutions G2L/P7L/W9L and the deletions H4-, E5-, and W11- and has preferably the amino acid sequence as shown in SEQ ID NO: 152 and/or is preferably encoded by the DNA sequence as shown in in SEQ ID NO: 153.

The term “Insulin growth factor 1 (IGF1) pro domain modified”, “modified IGF1 pro domain” or “IGF1-Pro-Modified” as used herein refers to the pro-peptide of IGF1 which is a naturally occurring amino acid sequence which does not have the function of a signal peptide in nature which is referred to in the Uniprot database as P05019 and in the Genbank database as NM_000618.4, NM_001111284.1 and NM_001111285.2 and which is modified by deletion of ten amino acid residues (VKMHTMSSSH) flanking 22-31 in the N-terminal end of pro peptide and has preferably the amino acid sequence as shown in SEQ ID NO: 142 and/or is preferably encoded by the DNA sequence as shown in in SEQ ID NO: 143.

The term “Intestinal-type alkaline phosphatase (ALPI) pro domain modified”, “modified ALPI pro domain “ALPI-Modified” or “ALPI-Pro-Modified” as used herein refers to the pro-peptide of ALPI which is a naturally occurring amino acid sequence which does not have the function of a signal peptide in nature which is referred to in the Uniprot database as P09923 and in the Genbank database as NM_001631.4 and which is modified by the substitutions A504L/A505L/S511L/G517L/T518L and deletions H506-, P507-, A509-, A510- and P513- and has preferably the amino acid sequence as shown in SEQ ID NO: 189 and/or is preferably encoded by the DNA sequence as shown in SEQ ID NO: 190.

The term “the mRNA comprises a nucleic acid sequence encoding the propeptide of IGF1, and a nucleic acid sequence encoding the mature IGF1 and does not comprise a nucleic acid sequence encoding an E-peptide of IGF1” as used herein refers usually to a mRNA which comprises a nucleotide sequence encoding the propeptide (also called pro-domain) of human IGF1 having 27 amino acids, and a nucleotide sequence encoding the mature human IGF1 having 70 amino acids and which does not comprise a nucleotide sequence encoding an E-peptide (also called E-domain) of human IGF1 i.e. does not comprise a nucleotide sequence encoding a Ea-, Eb- or Ec-domain. The nucleotide sequence encoding the propeptide (also called pro-domain) of human IGF1 having 27 amino acids, and the nucleotide sequence encoding the mature human IGF1 having 70 amino acids may be codon optimized.

The term “vector” or “expression vector” as used herein refers to naturally occurring or synthetically generated constructs for uptake, proliferation, expression or transmission of nucleic acids in a cell, e.g. plasmids, minicircles, phagemids, cosmids, artificial chromosomes/mini-chromosomes, bacteriophages, viruses such as baculovirus, retrovirus, adenovirus, adeno-associated virus, herpes simplex virus, bacteriophages. Vectors can either integrate into the genome of the host cell or remain as autonomously replicating construct within the host cell. Methods used to construct vectors are well known to a person skilled in the art and described in various publications. In particular techniques for constructing suitable vectors, including a description of the functional and regulatory components such as promoters, enhancers, termination and polyadenylation signals, selection markers, origins of replication, and splicing signals, are known to the person skilled in the art. The eukaryotic expression vectors will typically contain also prokaryotic sequences that facilitate the propagation of the vector in bacteria such as an origin of replication and antibiotic resistance genes for selection in bacteria which might be removed before transfection of eukaryotic cells. A variety of eukaryotic expression vectors, containing a cloning site into which a polynucleotide can be operably linked, are well known in the art and some are commercially available from companies such as Agilent Technologies, Santa Clara, Calif.; Invitrogen, Carlsbad, Calif.; Promega, Madison, Wis. or Invivogen, San Diego, Calif.

The term “gene therapy vector” as used herein refers to any vector that is being used to deliver a nucleic acid sequence e.g. a nucleic acid sequence coding for a gene into cells. Gene therapy vectors and methods of gene delivery are well known in the art. Non-limiting examples of these methods include viral vector delivery systems including DNA and RNA viruses, which have either episomal or integrated genomes after delivery to the cell, non-viral vector delivery systems including DNA plasmids, naked nucleic acid, and nucleic acid complexed with a delivery vehicle, transposon system (for delivery and integration into the host genomes; Moriarity, et al. (2013) Nucleic Acids Res 41(8), e92, Aronovich, et al., (2011) Hum. Mol. Genet. 20(R1), R14-R20), retrovirus-mediated DNA transfer (e.g., Moloney Mouse Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus; see e.g., Kay et al. (1993) Science 262, 117-119, Anderson (1992) Science 256, 808-813), and DNA virus-mediated DNA transfer including adenovirus, herpes virus, parvovirus and adeno-associated virus (e.g., Ali et al. (1994) Gene Therapy 1, 367-384). Viral vectors also include but are not limited to adeno-associated virus, adenoviral virus, lentivirus, retroviral, and herpes simplex virus vectors. Vectors capable of integration in the host genome include but are not limited to retrovirus or lentivirus.

The term “transcription unit”, “expression unit” or “expression cassette” as used herein refers a region within a vector, construct or polynucleotide sequence that contains one or more genes to be transcribed, wherein the genes contained within the segment are operably linked to each other. They are transcribed from a single promoter and transcription is terminated by at least one polyadenylation signal. As a result, the different genes are at least transcriptionally linked. More than one protein or product can be transcribed and expressed from each transcription unit (multicistronic transcription unit). Each transcription unit will comprise the regulatory elements necessary for the transcription and translation of any of the selected sequence that are contained within the unit and each transcription unit may contain the same or different regulatory elements. For example, each transcription unit may contain the same terminator. IRES element or introns may be used for the functional linking of the genes within a transcription unit. A vector or polynucleotide sequence may contain more than one transcription unit.

The term “skeletal muscle injury” as used herein refers to any injuries and ruptures of skeletal muscle, preferably ruptures of skeletal muscle, induced by eccentric muscle contractions, elongations and muscle overload. In principle any skeletal muscle can be affected by such injury or rupture. Preferably skeletal muscle injury are injuries and ruptures of skeletal muscle wherein the skeletal muscles are selected from the muscle groups of the head, the neck, the thorax, the back, the abdomen, the pelvis, the arms, the legs and the hip.

More preferably skeletal muscle injury are injuries and ruptures wherein the skeletal muscles are selected from the group consisting of plantaris, temporal, papillary, pectoralis major, tibialis posterior, tibialis anterior, gastrocnemius, coracobrachialis, diaphragma, palmaris longus, rectus abdominis, external anal sphincter, internal anal sphincter, subscapularis, biceps, triceps, quadriceps, calf, groin, hamstring, deltoid, teres major, rotator cuff supraspinatus, rotator cuff infraspinatus, rotator cuff teres minor, rotator cuff subscapularis, rectus femoralis, rectus abdominis, abdominal external oblique, masseter, trapezius, latissimus, pectoralis, erector spinae, iliocostalis, longissimus, spinalis, latissimus dorsi, transversospinales, semispinalis dorsi, semispinalis cervices, semispinalis capitis, multifidus, rotatores, interspinales, intertransversarii, splenius capitis, splenius cervices, intercostals, subcostales, transversus thoracis, levatores costarum, serratus posterior inferior, serratus posterior superior, transversus abdominis, rectus abdominis, pyramidalis, cremaster, quadratus lumborum, external oblique, internal oblique.

Even more preferably skeletal muscle injury are injuries and ruptures wherein the skeletal muscles are selected from the group consisting of plantaris, temporal, papillary, pectoralis major, tibialis posterior, tibialis anterior, gastrocnemius, coracobrachialis, diaphragma, palmaris longus, rectus abdominis, external anal sphincter, internal anal sphincter, subscapularis, biceps, triceps, quadriceps, calf, groin, hamstring, deltoid, teres major, rotator cuff supraspinatus, rotator cuff infraspinatus, rotator cuff teres minor, rotator cuff subscapularis, rectus femoralis, rectus abdominis, abdominal external oblique, masseter, trapezius, latissimus, pectoralis.

Preferably any injuries and ruptures of skeletal muscle, preferably ruptures of skeletal muscle, induced by eccentric muscle contraction, elongation or muscle overload are treated by the method of the present invention.

In a first aspect the present invention provides a mRNA comprising a nucleic acid sequence encoding a protein and a signal peptide, wherein the amino acids 1-9 of the N-terminal end of the amino acid sequence of the signal peptide have an average hydrophobic score of above 2, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

In one embodiment the mRNA comprises a nucleic acid sequence encoding a protein and a signal peptide, wherein the amino acids 1-9 of the N-terminal end of the amino acid sequence of the signal peptide have an average hydrophobic score of above 2, wherein the signal peptide is selected from the group consisting of

i) a signal peptide heterologous to said protein with the proviso that said protein is not an oxidoreductase or a signal peptide heterologous to said protein which is modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature or a naturally occurring amino acid sequence which does not have the function of a signal peptide in nature which is modified by insertion, deletion and/or substitution of at least one amino acid.

In a further embodiment the mRNA comprises a nucleic acid sequence encoding a protein and a signal peptide, wherein the amino acids 1-9 of the N-terminal end of the amino acid sequence of the signal peptide have an average hydrophobic score of above 2, wherein the signal peptide is selected from the group consisting of

i) a signal peptide heterologous to said protein, wherein the signal peptide heterologous to said protein is modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

In a further embodiment the mRNA comprises a nucleic acid sequence encoding a protein and a signal peptide, wherein the amino acids 1-9 of the N-terminal end of the amino acid sequence of the signal peptide have an average hydrophobic score of above 2, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is modified by insertion, deletion and/or substitution of at least one amino acid.

In a further embodiment the mRNA comprises a nucleic acid sequence encoding a protein and a signal peptide, wherein the amino acids 1-9 of the N-terminal end of the amino acid sequence of the signal peptide have an average hydrophobic score of above 2, wherein the signal peptide is selected from the group consisting of

i) a signal peptide heterologous to said protein, wherein the signal peptide heterologous to said protein is modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is modified by insertion, deletion and/or substitution of at least one amino acid.

In a further embodiment the mRNA comprises a nucleic acid sequence encoding a protein and a signal peptide, wherein the amino acids 1-9 of the N-terminal end of the amino acid sequence of the signal peptide have an average hydrophobic score of above 2, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase; and
ii) a signal peptide homologous to said protein, wherein the signal peptide homologous to said protein is modified by insertion, deletion and/or substitution of at least one amino acid.

In a further embodiment the mRNA comprises a nucleic acid sequence encoding a protein and a signal peptide, wherein the amino acids 1-9 of the N-terminal end of the amino acid sequence of the signal peptide have an average hydrophobic score of above 2, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase; and
iii) a naturally occurring amino acid sequence which does not have the function of a signal peptide in nature, wherein the naturally occurring amino acid sequence is optionally modified by insertion, deletion and/or substitution of at least one amino acid.

In a further embodiment the mRNA comprises a nucleic acid sequence encoding a protein and a signal peptide, wherein the amino acids 1-9 of the N-terminal end of the amino acid sequence of the signal peptide have an average hydrophobic score of above 2, wherein the signal peptide is selected from the group consisting of

ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

In a further embodiment the mRNA comprises a nucleic acid sequence encoding a protein and a signal peptide, wherein the amino acids 1-9 of the N-terminal end of the amino acid sequence of the signal peptide have an average hydrophobic score of above 2, wherein the signal peptide is a signal peptide heterologous to said protein, wherein the signal peptide heterologous to said protein is optionally modified by insertion, deletion and/or substitution of at least one amino acid, with the proviso that said protein is not an oxidoreductase.

In a further embodiment the mRNA comprises a nucleic acid sequence encoding a protein and a signal peptide, wherein the amino acids 1-9 of the N-terminal end of the amino acid sequence of the signal peptide have an average hydrophobic score of above 2, wherein the signal peptide is a signal peptide homologous to said protein, wherein the signal peptide homologous to said protein is modified by insertion, deletion and/or substitution of at least one amino acid.

In a further embodiment the mRNA comprises a nucleic acid sequence encoding a protein and a signal peptide, wherein the amino acids 1-9 of the N-terminal end of the amino acid sequence of the signal peptide have an average hydrophobic score of above 2, wherein the signal peptide is a naturally occurring amino acid sequence which does not have the function of a signal peptide in nature, wherein the naturally occurring amino acid sequence is optionally modified by insertion, deletion and/or substitution of at least one amino acid.

In a further embodiment the mRNA comprises a nucleic acid sequence encoding a protein and a signal peptide, wherein the amino acids 1-7 of the N-terminal end of the amino acid sequence of the signal peptide have an average hydrophobic score of above 1.5, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

In one embodiment the mRNA comprises a nucleic acid sequence encoding a protein and a signal peptide, wherein the amino acids 1-7 of the N-terminal end of the amino acid sequence of the signal peptide have an average hydrophobic score of above 1.5, wherein the signal peptide is selected from the group consisting of

i) a signal peptide heterologous to said protein with the proviso that said protein is not an oxidoreductase or a signal peptide heterologous to said protein which is modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature or a naturally occurring amino acid sequence which does not have the function of a signal peptide in nature which is modified by insertion, deletion and/or substitution of at least one amino acid.

In a further embodiment the mRNA comprises a nucleic acid sequence encoding a protein and a signal peptide, wherein the amino acids 1-7 of the N-terminal end of the amino acid sequence of the signal peptide have an average hydrophobic score of above 1.5, wherein the signal peptide is selected from the group consisting of

i) a signal peptide heterologous to said protein, wherein the signal peptide heterologous to said protein is modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

In a further embodiment the mRNA comprises a nucleic acid sequence encoding a protein and a signal peptide, wherein the amino acids 1-7 of the N-terminal end of the amino acid sequence of the signal peptide have an average hydrophobic score of above 1.5, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is modified by insertion, deletion and/or substitution of at least one amino acid.

In a further embodiment the mRNA comprises a nucleic acid sequence encoding a protein and a signal peptide, wherein the amino acids 1-7 of the N-terminal end of the amino acid sequence of the signal peptide have an average hydrophobic score of above 1.5, wherein the signal peptide is selected from the group consisting of

i) a signal peptide heterologous to said protein, wherein the signal peptide heterologous to said protein is modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is modified by insertion, deletion and/or substitution of at least one amino acid.

In a further embodiment the mRNA comprises a nucleic acid sequence encoding a protein and a signal peptide, wherein the amino acids 1-7 of the N-terminal end of the amino acid sequence of the signal peptide have an average hydrophobic score of above 1.5, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase; and
ii) a signal peptide homologous to said protein, wherein the signal peptide homologous to said protein is modified by insertion, deletion and/or substitution of at least one amino acid.

In further embodiment the mRNA comprises a nucleic acid sequence encoding a protein and a signal peptide, wherein the amino acids 1-7 of the N-terminal end of the amino acid sequence of the signal peptide have an average hydrophobic score of above 1.5, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase; and
iii) a naturally occurring amino acid sequence which does not have the function of a signal peptide in nature, wherein the naturally occurring amino acid sequence is optionally modified by insertion, deletion and/or substitution of at least one amino acid.

In further embodiment the mRNA comprises a nucleic acid sequence encoding a protein and a signal peptide, wherein the amino acids 1-7 of the N-terminal end of the amino acid sequence of the signal peptide have an average hydrophobic score of above 1.5, wherein the signal peptide is selected from the group consisting of

ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

In further embodiment the mRNA comprises a nucleic acid sequence encoding a protein and a signal peptide, wherein the amino acids 1-7 of the N-terminal end of the amino acid sequence of the signal peptide have an average hydrophobic score of above 1.5, wherein the signal peptide is a signal peptide heterologous to said protein, wherein the signal peptide heterologous to said protein is optionally modified by insertion, deletion and/or substitution of at least one amino acid, with the proviso that said protein is not an oxidoreductase.

In further embodiment the mRNA comprises a nucleic acid sequence encoding a protein and a signal peptide, wherein the amino acids 1-7 of the N-terminal end of the amino acid sequence of the signal peptide have an average hydrophobic score of above 1.5, wherein the signal peptide is a signal peptide homologous to said protein, wherein the signal peptide homologous to said protein is modified by insertion, deletion and/or substitution of at least one amino acid.

In a further embodiment the mRNA comprises a nucleic acid sequence encoding a protein and a signal peptide, wherein the amino acids 1-7 of the N-terminal end of the amino acid sequence of the signal peptide have an average hydrophobic score of above 1.5, wherein the signal peptide is a naturally occurring amino acid sequence which does not have the function of a signal peptide in nature, wherein the naturally occurring amino acid sequence is optionally modified by insertion, deletion and/or substitution of at least one amino acid.

In a further embodiment the mRNA comprises a nucleic acid sequence encoding a protein and a signal peptide, wherein the amino acids 1-5 of the N-terminal end of the amino acid sequence of the signal peptide have an average hydrophobic score of above 1.3, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

In one embodiment the mRNA comprises a nucleic acid sequence encoding a protein and a signal peptide, wherein the amino acids 1-5 of the N-terminal end of the amino acid sequence of the signal peptide have an average hydrophobic score of above 1.3, wherein the signal peptide is selected from the group consisting of

i) a signal peptide heterologous to said protein with the proviso that said protein is not an oxidoreductase or a signal peptide heterologous to said protein which is modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature or a naturally occurring amino acid sequence which does not have the function of a signal peptide in nature which is modified by insertion, deletion and/or substitution of at least one amino acid.

In a further embodiment the mRNA comprises a nucleic acid sequence encoding a protein and a signal peptide, wherein the amino acids 1-5 of the N-terminal end of the amino acid sequence of the signal peptide have an average hydrophobic score of above 1.3, wherein the signal peptide is selected from the group consisting of

i) a signal peptide heterologous to said protein, wherein the signal peptide heterologous to said protein is modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

In a further embodiment the mRNA comprises a nucleic acid sequence encoding a protein and a signal peptide, wherein the amino acids 1-5 of the N-terminal end of the amino acid sequence of the signal peptide have an average hydrophobic score of above 1.3, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is modified by insertion, deletion and/or substitution of at least one amino acid.

In a further embodiment the mRNA comprises a nucleic acid sequence encoding a protein and a signal peptide, wherein the amino acids 1-5 of the N-terminal end of the amino acid sequence of the signal peptide have an average hydrophobic score of above 1.3, wherein the signal peptide is selected from the group consisting of

i) a signal peptide heterologous to said protein, wherein the signal peptide heterologous to said protein is modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is modified by insertion, deletion and/or substitution of at least one amino acid.

In one embodiment the mRNA comprises a nucleic acid sequence encoding a protein and a signal peptide, wherein the amino acids 1-5 of the N-terminal end of the amino acid sequence of the signal peptide have an average hydrophobic score of above 1.3, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase; and
ii) a signal peptide homologous to said protein, wherein the signal peptide homologous to said protein is modified by insertion, deletion and/or substitution of at least one amino acid.

In further embodiment the mRNA comprises a nucleic acid sequence encoding a protein and a signal peptide, wherein the amino acids 1-5 of the N-terminal end of the amino acid sequence of the signal peptide have an average hydrophobic score of above 1.3, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase; and
iii) a naturally occurring amino acid sequence which does not have the function of a signal peptide in nature, wherein the naturally occurring amino acid sequence is optionally modified by insertion, deletion and/or substitution of at least one amino acid.

In further embodiment the mRNA comprises a nucleic acid sequence encoding a protein and a signal peptide, wherein the amino acids 1-5 of the N-terminal end of the amino acid sequence of the signal peptide have an average hydrophobic score of above 1.3, wherein the signal peptide is selected from the group consisting of

ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

In further embodiment the mRNA comprises a nucleic acid sequence encoding a protein and a signal peptide, wherein the amino acids 1-5 of the N-terminal end of the amino acid sequence of the signal peptide have an average hydrophobic score of above 1.3, wherein the signal peptide is a signal peptide heterologous to said protein, wherein the signal peptide heterologous to said protein is optionally modified by insertion, deletion and/or substitution of at least one amino acid, with the proviso that said protein is not an oxidoreductase.

In further embodiment the mRNA comprises a nucleic acid sequence encoding a protein and a signal peptide, wherein the amino acids 1-5 of the N-terminal end of the amino acid sequence of the signal peptide have an average hydrophobic score of above 1.3, wherein the signal peptide is a signal peptide homologous to said protein, wherein the signal peptide homologous to said protein is modified by insertion, deletion and/or substitution of at least one amino acid.

In a further embodiment the mRNA comprises a nucleic acid sequence encoding a protein and a signal peptide, wherein the amino acids 1-5 of the N-terminal end of the amino acid sequence of the signal peptide have an average hydrophobic score of above 1.3, wherein the signal peptide is a naturally occurring amino acid sequence which does not have the function of a signal peptide in nature, wherein the naturally occurring amino acid sequence is optionally modified by insertion, deletion and/or substitution of at least one amino acid.

In a preferred embodiment the mRNA comprises a nucleic acid sequence encoding a protein and a signal peptide, wherein the signal peptide comprises or consists of an amino acid sequence of between 16 and 40 amino acids in length, wherein the amino acids 1-9 of the N-terminal end of said amino acid sequence have an average hydrophobic score of above 2, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

In a further preferred embodiment the mRNA comprises a nucleic acid sequence encoding a protein and a signal peptide, wherein the signal peptide comprises or consists of an amino acid sequence of between 16 and 40 amino acids in length, wherein the amino acids 1-9 of the N-terminal end of said amino acid sequence have an average hydrophobic score of above 2, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase; and
ii) a signal peptide homologous to said protein, wherein the signal peptide homologous to said protein is modified by insertion, deletion and/or substitution of at least one amino acid.

In a further preferred embodiment the mRNA comprises a nucleic acid sequence encoding a protein and a signal peptide, wherein the signal peptide comprises or consists of an amino acid sequence of between 16 and 40 amino acids in length, wherein the amino acids 1-9 of the N-terminal end of said amino acid sequence have an average hydrophobic score of above 2, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase; and
iii) a naturally occurring amino acid sequence which does not have the function of a signal peptide in nature, wherein the naturally occurring amino acid sequence is optionally modified by insertion, deletion and/or substitution of at least one amino acid.

In a further preferred embodiment the mRNA comprises a nucleic acid sequence encoding a protein and a signal peptide, wherein the signal peptide comprises or consists of an amino acid sequence of between 16 and 40 amino acids in length, wherein the amino acids 1-9 of the N-terminal end of said amino acid sequence have an average hydrophobic score of above 2, wherein the signal peptide is selected from the group consisting of

ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

In a further preferred embodiment the mRNA comprises a nucleic acid sequence encoding a protein and a signal peptide, wherein the signal peptide comprises or consists of an amino acid sequence of between 16 and 40 amino acids in length, wherein the amino acids 1-9 of the N-terminal end of said amino acid sequence have an average hydrophobic score of above 2, wherein the signal peptide is a signal peptide heterologous to said protein, wherein the signal peptide heterologous to said protein is optionally modified by insertion, deletion and/or substitution of at least one amino acid, with the proviso that said protein is not an oxidoreductase.

In a further preferred embodiment the mRNA comprises a nucleic acid sequence encoding a protein and a signal peptide, wherein the signal peptide comprises or consists of an amino acid sequence of between 16 and 40 amino acids in length, wherein the amino acids 1-9 of the N-terminal end of said amino acid sequence have an average hydrophobic score of above 2, wherein the signal peptide is a signal peptide homologous to said protein, wherein the signal peptide homologous to said protein is modified by insertion, deletion and/or substitution of at least one amino acid.

In a further preferred embodiment the mRNA comprises a nucleic acid sequence encoding a protein and a signal peptide, wherein the signal peptide comprises or consists of an amino acid sequence of between 16 and 40 amino acids in length, wherein the amino acids 1-9 of the N-terminal end of said amino acid sequence have an average hydrophobic score of above 2, wherein the signal peptide is a naturally occurring amino acid sequence which does not have the function of a signal peptide in nature, wherein the naturally occurring amino acid sequence is optionally modified by insertion, deletion and/or substitution of at least one amino acid.

In one embodiment the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid, wherein the modification by insertion, deletion and/or substitution of at least one amino acid is made within the amino acids 1-11, preferably within the amino acids 1-10, more preferably within the amino acids 1-9, even more preferably within the amino acids 1-8, in particular within the amino acids 1-7, more particular within the amino acids 1-6, even more particular within the amino acids 1-5, particular preferably within the amino acids 1-4, more particular preferably within the amino acids 1-3, even more particular preferably within the amino acids 1-2 of the N-terminal end of the amino acid sequence of the signal peptide.

The term “hydrophobic score” or “hydrophobicity score” is used synonymously to the term “hydropathy score” herein and refers to the degree of hydrophobicity of an amino acid as calculated according to the Kyte-Doolittle scale (Kyte J., Doolittle R. F.; J. Mol. Biol. 157:105-132(1982)). The amino acid hydrophobic scores according to the Kyte-Doolittle scale are as follows:

| | One Letter | Hydrophobic |
|---|---|---|
| Amino Acid | Code | Score |
| Isoleucine | I | 4.5 |
| Valine | V | 4.2 |
| Leucine | L | 3.8 |
| Phenylalanine | F | 2.8 |
| Cysteine | C | 2.5 |
| Methionine | M | 1.9 |
| Alanine | A | 1.8 |
| Glycine | G | −0.4 |
| Threonine | T | −0.7 |
| Serine | S | −0.8 |
| Tryptophan | W | −0.9 |
| Tyrosine | Y | −1.3 |
| Proline | P | −1.6 |
| Histidine | H | −3.2 |
| Glutamic acid | E | −3.5 |
| Glutamine | Q | −3.5 |
| Aspartic acid | D | −3.5 |
| Asparagine | N | −3.5 |
| Lysine | K | −3.9 |
| Arginine | R | −4.5 |

The “average hydrophobic score” of an amino acid sequence e.g. the average hydrophobic score of the amino acids 1-9 of the N-terminal end of the amino acid sequence of a signal peptide is calculated by adding the hydrophobic score according to the Kyte-Doolittle scale of each of the amino acid of the amino acid sequence e.g. the hydrophobic score of each of the nine amino acids of the amino acids 1-9 of the N-terminal end, divided by the number of the amino acids, e.g divided by nine.

In one embodiment of the present invention the amino acids 1-9 of the N-terminal end of the amino acid sequence have an average hydrophobic score of equal to or above 2.05, preferably of equal to or above 2.1, more preferably of equal to or above 2.15, even more preferably of equal to or above 2.2, in particular of equal to or above 2.25, more particular of equal to or above 2.3, even more particular of equal to or above 2.35. In a further embodiment the amino acids 1-9 of the N-terminal end of the amino acid sequence have an average hydrophobic score of between 2.05 and 4.5, preferably between 2.1 and 4.5, more preferably between 2.15 and 4.5, even more preferably between 2.2 and 4.5, in particular between 2.25 and 4.5, more particular between 2.3 and 4.5, even more particular between 2.35 and 4.5. In a further embodiment the amino acids 1-9 of the N-terminal end of the amino acid sequence have an average hydrophobic score of between 2.05 and 4.0, preferably between 2.1 and 4.0, more preferably between 2.15 and 4.0, even more preferably between 2.2 and 4.0, in particular between 2.25 and 4.0, more particular between 2.3 and 4.0, even more particular between 2.35 and 4.0.

In one embodiment of the present invention the average hydrophobic score of the last nine amino acids of the C-terminal end of the amino acid sequence of the signal peptide is at least 1.0 unit below, preferably at least 1.1 units below, more preferably at least 1.2 units below, even more preferably at least 1.3 units below, in particular between 1.0 and 4 units below, more particular between 1.1 and 4 units below, even more particular between 1.2 and 4 units below, most particular between 1.3 and 4 units below the average hydrophobic score of the amino acids 1-9 of the N-terminal end of the amino acid sequence of the signal peptide.

In one embodiment of the present invention the amino acids 1-9, the amino acids 2-10, the amino acids 3-11, the amino acids 4-12 and the amino acids 5-13 of the N-terminal end of the amino acid sequence of the signal peptide, have each an average hydrophobic score of above 1.5, preferably an average hydrophobic score of above 1.6, more preferably an average hydrophobic score of above 1.7. even more preferably an average hydrophobic score of above 1.8, much more preferably an average hydrophobic score of above 1.9, in particular an average hydrophobic score of between 1.5 and 4.5, more particular an average hydrophobic score of between 1.6 and 4.5, even more particular an average hydrophobic score of between 1.7 and 4.5, much more particular an average hydrophobic score of between 1.8 and 4.5, most particular an average hydrophobic score of between 1.9 and 4.5.

In one embodiment of the present invention the average hydrophobic score of the amino acids 8-16 of the N-terminal end of the amino acid sequence of the signal peptide is at least equal to or lower, preferably at least 0.4 units lower, more preferably between 0.4 and 2.0 units lower, than the average hydrophobic score of the amino acids 3-11 of the N-terminal end of the amino acid sequence of the signal peptide.

In one embodiment of the present invention the signal peptide comprises or consists of an amino acid sequence of between 18 and 40 amino acids in length and wherein the average hydrophobic score of the amino acids 10-18 of the N-terminal end of the amino acid sequence of the signal peptide is at least 0.5 units, preferably between 0.5 and 3.0 units, below the average hydrophobic score of the amino acids 3-11 of the N-terminal end of the amino acid sequence of the signal peptide.

In one embodiment of the present invention the average hydrophobic score of the last nine amino acids of the C-terminal end of the amino acid sequence of the signal peptide is at least 1.5 units, preferably between 1.5 and 3.5 units, below the average hydrophobic score of the amino acids 3-11 of the N-terminal end of the amino acid sequence of the signal peptide.

In one embodiment of the present invention the average hydrophobic score of any 9 consecutive amino acids of the amino acid sequence of the signal peptide does not exceed 4.1.

In one embodiment of the present invention the last nine amino acids of the C-terminal end of the amino acid sequence of the signal peptide comprise at least one amino acid with negative hydrophobicity score, preferably the last nine amino acids of the C-terminal end of the amino acid sequence of the signal peptide comprise an amino acid selected from the group consisting of G, Q, N, T, S, R, K, H, D, E, P, Y and W.

In one embodiment of the present invention the second amino acid of the amino acids 1-9 of the N-terminal end of the amino acid sequence of the signal peptide is selected from the group consisting of P, Y, W, S, T, G, A, M, C, F, L, V and I.

In a preferred embodiment of the present invention the second amino acid of the amino acids 1-9 of the N-terminal end of the amino acid sequence of the signal peptide is selected from the group consisting of A, L, S, T, V and W.

In one embodiment of the present invention the amino acids 1-9 of the N-terminal end of the amino acid sequence of the signal peptide have an average polarity of 6.1 or below, preferably an average polarity of below 6.1, more preferably an average polarity of below 4, even more preferably an average polarity of below 2, in particular an average polarity of between 6.1 and 0, more particular an average polarity of between 4 and 0, even more particular an average polarity of between 2 and 0, most particular an average polarity of between 1 and 0.2.

In one embodiment of the present invention the amino acids 1-7 of the N-terminal end of the amino acid sequence of the signal peptide have an average polarity of 6.1 or below, preferably an average polarity of below 6.1, more preferably an average polarity of below 4, even more preferably an average polarity of below 2, in particular an average polarity of between 6.1 and 0, more particular an average polarity of between 4 and 0, even more particular an average polarity of between 2 and 0, most particular an average polarity of between 1 and 0.2.

In one embodiment of the present invention the amino acids 1-5 of the N-terminal end of the amino acid sequence of the signal peptide have an average polarity of 6.1 or below, preferably an average polarity of below 6.1, more preferably an average polarity of below 4, even more preferably an average polarity of below 2, in particular an average polarity of between 6.1 and 0, more particular an average polarity of between 4 and 0, even more particular an average polarity of between 2 and 0, most particular an average polarity of between 1.1 and 0.2.

The polarity is calculated according to Zimmerman Polarity index (Zimmerman J. M., Eliezer N., Simha R.; J. Theor. Biol. 21:170-201(1968)). The “average polarity” of an amino acid sequence e.g. the average polarity of the amino acids 1-9 of the N-terminal end of the amino acid sequence of a signal peptide is calculated by adding the polarity value calculated according to Zimmerman Polarity index of each of the amino acid of the amino acid sequence e.g. the average polarity of each of the nine amino acids of the amino acids 1-9 of the N-terminal end, divided by the number of the amino acids, e.g divided by nine. The polarity of amino acids according to Zimmerman Polarity index is as follows:

| | One Letter | |
|---|---|---|
| Amino Acid | Code | Polarity |
| Isoleucine | I | 0.13 |
| Valine | V | 0.13 |
| Leucine | L | 0.13 |
| Phenylalanine | F | 0.35 |
| Cysteine | C | 1.48 |
| Methionine | M | 1.43 |
| Alanine | A | 0 |
| Glycine | G | 0 |
| Threonine | T | 1.66 |
| Serine | S | 1.67 |
| Tryptophan | W | 2.1 |
| Tyrosine | Y | 1.61 |
| Proline | P | 1.58 |
| Histidine | H | 51.6 |
| Glutamic acid | E | 49.9 |
| Glutamine | Q | 3.53 |
| Aspartic acid | D | 49.7 |
| Asparagine | N | 3.38 |
| Lysine | K | 49.5 |
| Arginine | R | 52 |

The above mentioned average hydrophobic score or average polarity of an amino acid sequence of a signal peptide of the present invention can be calculated by using the publicly available online database ProtScale (http://www.expasy.org/tools/protscale.html) referred to in Gasteiger E. et al. (Gasteiger E., Hoogland C., Gattiker A., Duvaud S., Wilkins M. R., Appel R. D., Bairoch A.; Protein Identification and Analysis Tools on the ExPASy Server; (In) John M. Walker (ed): The Proteomics Protocols Handbook, Humana Press (2005).pp. 571-607) with the selection of Hydrophobicity of Kyte & Doolittle scale (“Hphob./Kyte & Doolittle”) or polarity of Zimmerman scale (“Polarity/Zimmerman”) and settings corresponding to a specific window size (e.g. window size of 9 amino acids) of a signal peptide, with the window edge relative weight value set to 100%, and without scale normalization. The respective numerical value data can be retrieved by opening link on ‘Numerical format (verbose)’ in the result page.

In one embodiment of the present invention the mRNA comprises a nucleic acid sequence encoding a protein and a signal peptide, wherein the amino acids 1-7 of the N-terminal end of the amino acid sequence of the signal peptide have an average hydrophobic score of above 1.7. In one embodiment the mRNA comprises a nucleic acid sequence encoding a protein and a signal peptide, wherein the amino acids 1-7 of the N-terminal end of the amino acid sequence of the signal peptide have an average hydrophobic score of above 1.7 and the signal peptide comprises or consists of an amino acid sequence of between 14 and 40 amino acid length.

In one embodiment of the present invention the amino acids 1-7 of the N-terminal end of the amino acid sequence have an average hydrophobic score of equal or above 1.6, preferably of equal to or above 1.7, more preferably of equal to or above 1.75, even more preferably of equal to or above 1.8, in particular of equal to or above 2.0, more particular of equal to or above 2.1, even more particular of equal to or above 2.2. In a further embodiment the amino acids 1-7 of the N-terminal end of the amino acid sequence have an average hydrophobic score of between 1.6 and 4.5, preferably between 1.7 and 4.5, more preferably between 1.75 and 4.5, even more preferably between 1.8 and 4.5, in particular between 2.0 and 4.5, more particular between 2.1 and 4.5, even more particular between 2.2 and 4.5. In a further embodiment the amino acids 1-7 of the N-terminal end of the amino acid sequence have an average hydrophobic score of between 1.6 and 4.0, preferably between 1.7 and 4.0, more preferably between 1.75 and 4.0, even more preferably between 1.8 and 4.0, in particular between 2.0 and 4.0, more particular between 2.1 and 4.0, even more particular between 2.2 and 4.0.

In one embodiment of the present invention the average hydrophobic score of the last seven amino acids of the C-terminal end of the amino acid sequence of the signal peptide is equal to or below the average hydrophobic score of the amino acids 1-7 of the N-terminal end of the amino acid sequence of the signal peptide, preferably at least 0.06 units below, more preferably at least 1.0 unit below, even more preferably at least 1.1 units below, in particular at least 1.2 units below, more particular between 1.0 and 4 units below, even more particular between 1.0 and 4 units below, most particular between 1.2 and 4 units below.

In one embodiment of the present invention the amino acids 1-7, the amino acids 2-8, the amino acids 3-9, the amino acids 4-10 and the amino acids 5-11 of the N-terminal end of the amino acid sequence of the signal peptide, have each an average hydrophobic score of above 1.4, preferably an average hydrophobic score of above 1.5, more preferably an average hydrophobic score of above 1.6. even more preferably an average hydrophobic score of above 1.7, much more preferably an average hydrophobic score of above 1.75, in particular an average hydrophobic score of between 1.4 and 4.5, more particular an average hydrophobic score of between 1.5 and 4.5, even more particular an average hydrophobic score of between 1.6 and 4.5, much more particular an average hydrophobic score of between 1.7 and 4.5, most particular an average hydrophobic score of between 1.75 and 4.5.

In one embodiment of the present invention the average hydrophobic score of the last seven amino acids of the C-terminal end of the amino acid sequence of the signal peptide is at least 1.0 units, preferably between 1.0 and 3.6 units, below the average hydrophobic score of the amino acids 3-9 of the N-terminal end of the amino acid sequence of the signal peptide.

In one embodiment of the present invention the average hydrophobic score of any 7 consecutive amino acids of the amino acid sequence of the signal peptide does not exceed 4.1.

In one embodiment of the present invention the last seven amino acids of the C-terminal end of the amino acid sequence of the signal peptide comprise at least one amino acid with negative hydrophobicity score, preferably the last seven amino acids of the C-terminal end of the amino acid sequence of the signal peptide comprise an amino acid selected from the group consisting of G, Q, N, T, S, R, K, H, D, E, P, Y and W.

In one embodiment of the present invention the second amino acid of the amino acids 1-7 of the N-terminal end of the amino acid sequence of the signal peptide is selected from the group consisting of P, Y, W, S, T, G, A, M, C, F, L, V and I.

In a preferred embodiment of the present invention the second amino acid of the amino acids 1-7 of the N-terminal end of the amino acid sequence of the signal peptide is selected from the group consisting of A, L, S, T, V and W.

In one embodiment of the present invention the mRNA comprises a nucleic acid sequence encoding a protein and a signal peptide, wherein the amino acids 1-5 of the N-terminal end of the amino acid sequence of the signal peptide have an average hydrophobic score of above 1.3 units. In one embodiment the mRNA comprises a nucleic acid sequence encoding a protein and a signal peptide, wherein the amino acids 1-5 of the N-terminal end of the amino acid sequence of the signal peptide have an average hydrophobic score of above 1.3 and the signal peptide comprises or consists of an amino acid sequence of between 12 and 40 amino acid length.

In one embodiment of the present invention the amino acids 1-5 of the N-terminal end of the amino acid sequence have an average hydrophobic score of equal to or above 1.0, preferably of equal to or above 1.1, more preferably of equal to or above 1.2, even more preferably of equal to or above 1.25, in particular of equal to or above 1.3, more particular of equal to or above 1.35, even more particular of equal to or above 1.38. In a further embodiment the amino acids 1-5 of the N-terminal end of the amino acid sequence have an average hydrophobic score of between 1 and 4.5, preferably between 1.1 and 4.5, more preferably between 1.2 and 4.5, even more preferably between 1.25 and 4.5, in particular between 1.3 and 4.5, more particular between 1.35 and 4.5, even more particular between 1.38 and 4.5. In a further embodiment the amino acids 1-5 of the N-terminal end of the amino acid sequence have an average hydrophobic score of between 1.0 and 4.0, preferably between 1.1 and 4.0, more preferably between 1.2 and 4.0, even more preferably between 1.25 and 4.0, in particular between 1.3 and 4.0, more particular between 1.35 and 4.0, even more particular between 1.38 and 4.0.

In one embodiment of the present invention the average hydrophobic score of the last five amino acids of the C-terminal end of the amino acid sequence of the signal peptide is at least 0.2 units below, preferably at least 0.24 units below, more preferably at least 1.0 unit below, even more preferably at least 1.2 units below, in particular between 0.2 and 4 units below, more particular between 0.24 and 4 units below, even more particular between 1.0 and 4 units below, most particular between 1.2 and 4 units below the average hydrophobic score of the amino acids 1-5 of the N-terminal end of the amino acid sequence of the signal peptide.

In one embodiment of the present invention the amino acids 1-5, the amino acids 2-6, the amino acids 3-7, the amino acids 4-8 and the amino acids 5-9 of the N-terminal end of the amino acid sequence of the signal peptide, have each an average hydrophobic score of above 1.0, preferably an average hydrophobic score of above 1.15, more preferably an average hydrophobic score of above 1.2. even more preferably an average hydrophobic score of above 1.21, much more preferably an average hydrophobic score of above 1.23, in particular an average hydrophobic score of between 1.0 and 4.5, more particular an average hydrophobic score of between 1.15 and 4.5, even more particular an average hydrophobic score of between 1.2 and 4.5, much more particular an average hydrophobic score of between 1.21 and 4.5, most particular an average hydrophobic score of between 1.23 and 4.5.

In one embodiment of the present invention the average hydrophobic score of the last five amino acids of the C-terminal end of the amino acid sequence of the signal peptide is at least 1.2 units, preferably between 1.2 and 3.0 units, more preferably between 1.2 and 4.3 units below the average hydrophobic score of the amino acids 3-7 of the N-terminal end of the amino acid sequence of the signal peptide.

In one embodiment of the present invention the average hydrophobic score of any 5 consecutive amino acids of the amino acid sequence of the signal peptide does not exceed 4.2, preferably does not exceed 4.3.

In one embodiment of the present invention the five nine amino acids of the C-terminal end of the amino acid sequence of the signal peptide comprise at least one amino acid with negative hydrophobicity score, preferably the last five amino acids of the C-terminal end of the amino acid sequence of the signal peptide comprise an amino acid selected from the group consisting of G, Q, N, T, S, R, K, H, D, E, P, Y and W.

In one embodiment of the present invention the second amino acid of the amino acids 1-9 of the N-terminal end of the amino acid sequence of the signal peptide is selected from the group consisting of P, Y, W, S, T, G, A, M, C, F, L, V and I.

In a preferred embodiment of the present invention the second amino acid of the amino acids 1-9 of the N-terminal end of the amino acid sequence of the signal peptide is selected from the group consisting of A, L, S, T, V and W.

In one embodiment of the present invention the signal peptide is i) a signal peptide heterologous to said protein, wherein the signal peptide heterologous to said protein is optionally modified by insertion, deletion and/or substitution of less than 50% of the number of the amino acids of the amino acid sequence of the signal peptide heterologous to said protein.

In one embodiment of the present invention the signal peptide is i) a signal peptide heterologous to said protein, wherein the signal peptide heterologous to said protein is optionally modified by insertion, deletion and/or substitution of at least one amino acid, wherein the modified signal peptide has an amino acid sequence which differs from the amino acid sequence of the signal peptide heterologous to said protein without modification by one, preferably by two, more preferably by three, even more preferably by four, most preferably by five, in particular by six, more particular by seven, even more particular by eight, most particular by nine or ten amino acids.

In one embodiment, the modified signal peptide has an amino acid sequence which differs from the amino acid sequence of the signal peptide heterologous to said protein without modification by between 1-2 amino acids, preferably by between 1-3 amino acids, more preferably by between 1-4 amino acids, even more preferably by between 1-5 amino acids, most preferably by between 1-6 amino acids, in particular by between 1-7 amino acids, more particular by between 1-10 amino acids, even more particular by between 1-12 amino acids, most particular by between 1-15 amino acids.

In one embodiment, the modified signal peptide has a sequence identity of between 95% and 50%, preferably between 95% and 60%, more preferably between 95% and 70%, even more preferably between 95% and 80%, most preferably between 95% and 90% to the amino acid sequence of the signal peptide heterologous to said protein without modification.

In one embodiment of the present invention the signal peptide is i) a signal peptide heterologous to said protein, wherein the signal peptide heterologous to said protein is modified by insertion, deletion and/or substitution of at least one amino acid, with the proviso that the modified signal peptide has an amino acid sequence which differs from the amino acid sequence of the naturally occurring (homologous) signal peptide of said protein, by at least one, preferably by at least two, more preferably by at least three, even more preferably by at least four, most preferably by at least five, in particular by at least six, more particular by at least seven, even more particular by at least eight, most particular by at least nine or ten amino acids. In one embodiment the modified signal peptide heterologous to said protein has a sequence identity with the amino acid sequence of the naturally occurring (homologous) signal peptide of said protein of less than 95%, preferably of less than 90%, more preferably of less than 80%, even more preferably of less than 70%, most preferably of less than 60%, in particular of less than 50%.

In one embodiment of the present invention the signal peptide is ii) a signal peptide homologous to said protein, wherein the signal peptide homologous to said protein is modified by insertion, deletion and/or substitution of at least one amino acid, wherein the amino acids 1-9 of the N-terminal end of the amino acid sequence of the signal peptide homologous to said protein without modification have an average hydrophobic score of 2 and below, preferably of below 2.

In one embodiment of the present invention the signal peptide is ii) a signal peptide homologous to said protein, wherein the signal peptide homologous to said protein is modified by insertion, deletion and/or substitution of less than 50% of the number of the amino acids of the amino acid sequence of the signal peptide homologous to said protein.

In one embodiment of the present invention the signal peptide is ii) a signal peptide homologous to said protein, wherein the signal peptide homologous to said protein is modified by insertion, deletion and/or substitution of at least one amino acid, wherein the modified signal peptide homologous to said protein differs from the amino acid sequence of the signal peptide homologous to said protein without modification by one, preferably by two, more preferably by three, even more preferably by four, most preferably by five, in particular by six, more particular by seven, even more particular by eight to 12, most particular by nine to fifteen amino acids. In one embodiment, the modified signal peptide homologous to said protein differs from the amino acid sequence of the signal peptide homologous to said protein without modification by between 1-2 amino acids, preferably by between 1-3 amino acids, more preferably by between 1-4 amino acids, even more preferably by between 1-5 amino acids, most preferably by between 1-6 amino acids, in particular by between 1-7 amino acids, more particular by between 1-1-10 amino acids, even more particular by between 1-12 amino acids, most particular by between 1-15 amino acids.

In one embodiment the modified signal peptide homologous to said protein has a sequence identity of less than 95%, preferably of less than 90%, more preferably of less than 80%, even more preferably of less than 70%, most preferably of less than 60%, in particular of less than 50% to the amino acid sequence of the signal peptide homologous to said protein without modification.

In one embodiment of the present invention the signal peptide is iii) a naturally occurring amino acid sequence which does not have the function of a signal peptide in nature, wherein the naturally occurring amino acid sequence is optionally modified by insertion, deletion and/or substitution of less than 50% of the number of the amino acids of the amino acid sequence of the naturally occurring amino acid sequence.

In one embodiment of the present invention the signal peptide is iii) a naturally occurring amino acid sequence which does not have the function of a signal peptide in nature, wherein the naturally occurring amino acid sequence is modified by insertion, deletion and/or substitution of at least one amino acid, wherein the modified naturally occurring amino acid sequence differs from the amino acid sequence of the naturally occurring amino acid sequence without modification by one, preferably by two, more preferably by three, even more preferably by four, most preferably by five, in particular by six, more particular by seven, even more particular by eight to twelve, most particular by nine to fifteen amino acids. In one embodiment, the modified naturally occurring amino acid sequence differs from the amino acid sequence of the naturally occurring amino acid sequence without modification by between 1-2 amino acids, preferably by between 1-3 amino acids, more preferably by between 1-4 amino acids, even more preferably by between 1-5 amino acids, most preferably by between 1-6 amino acids, in particular by between 1-7 amino acids, more particular by between 1-1-10 amino acids, even more particular by between 1-12 amino acids, most particular by between 1-15 amino acids.

In one embodiment the modified naturally occurring amino acid sequence has a sequence identity of less than 95%, preferably of less than 90%, more preferably of less than 80%, even more preferably of less than 70%, most preferably of less than 60%, in particular of less than 50% to the amino acid sequence of the naturally occurring amino acid sequence without modification.

In one embodiment of the present invention the modified naturally occurring amino acid sequence which does not have the function of a signal peptide in nature has an amino acid sequence which differs from the amino acid sequence of a naturally occurring signal peptide by more than 50%, preferably by more than 60%, more preferably by more than 70%, even more preferably by more than 80%, most preferably by more than 90%, in particular by more than 95%, In one embodiment the modified naturally occurring amino acid sequence which does not have the function of a signal peptide in nature has a sequence identity with the amino acid sequence of a naturally occurring signal of less than 100%, preferably less than 95%, more preferably of less than 90%, even more preferably of less than 80%, most preferably of less than 70%, in particular of less than 60%, more particular of less than 50%.

In one embodiment of the present invention the signal peptide is i) a signal peptide heterologous to said protein, wherein the signal peptide heterologous to said protein is selected from the group consisting of the signal peptide of brain-derived neurotrophic factor (BDNF), the signal peptide of neurotrophin-3 (NTF-3), the signal peptide of fibroblast growth factor 5 (FGF5), the signal peptide of insulin-like growth factor-binding protein 5 (IBP5), the signal peptide of prostate and testis expressed protein 2 (PATE2), the signal peptide of extracellular superoxide dismutase (SOD3), and the signal peptide of complement factor H-related protein 2 (FHR2) or is i) a signal peptide heterologous to said protein modified by insertion, deletion and/or substitution of at least one amino acid, wherein the signal peptide heterologous to said protein is selected from the group consisting of the signal peptide of C-X-C Motif Chemokine Ligand 12 (CXCL12), the signal peptide of insulin growth factor 2 (IGF2), the signal peptide of insulin (INS), and the signal peptide of brain-derived neurotrophic factor (BDNF).

In one embodiment of the present invention the signal peptide is i) a signal peptide heterologous to said protein wherein the signal peptide heterologous to said protein is selected from the group consisting of the signal peptide of brain-derived neurotrophic factor (BDNF) as shown in SEQ ID NO:30, the signal peptide of neurotrophin-3 (NTF-3) as shown in SEQ ID NO:102, the signal peptide of fibroblast growth factor 5 (FGF5) as shown in SEQ ID NO:87, the signal peptide of insulin-like growth factor-binding protein 5 (IBP5) as shown in SEQ ID NO:97, the signal peptide of prostate and testis expressed protein 2 (PATE2) as shown in SEQ ID NO:107, the signal peptide of extracellular superoxide dismutase (SOD3) as shown in SEQ ID NO:112, and the signal peptide of complement factor H-related protein 2 (FHR2) as shown in SEQ ID NO:92 or is i) a signal peptide heterologous to said protein modified by insertion, deletion and/or substitution of at least one amino acid, wherein the modified signal peptide heterologous to said protein is selected from the group consisting of the modified signal peptide of C-X-C Motif Chemokine Ligand 12 (CXCL12) as shown in SEQ ID NO:132, the modified signal peptide of insulin growth factor 2 (IGF2) as shown in SEQ ID NO:127, the modified signal peptide of insulin (INS) as shown in SEQ ID NO:147, and the modified signal peptide of brain-derived neurotrophic factor (BDNF) as shown in SEQ ID NO:137.

In one embodiment of the present invention the signal peptide is i) a signal peptide heterologous to said protein wherein the signal peptide heterologous to said protein is selected from the group consisting of the signal peptide of brain-derived neurotrophic factor (BDNF), the signal peptide of neurotrophin-3 (NTF-3), the signal peptide of fibroblast growth factor 5 (FGF5), the signal peptide of insulin-like growth factor-binding protein 5 (IBP5), the signal peptide of prostate and testis expressed protein 2 (PATE2), the signal peptide of extracellular superoxide dismutase (SOD3), and the signal peptide of complement factor H-related protein 2 (FHR2). Preferably the signal peptide heterologous to said protein is selected from the group consisting of the signal peptide of brain-derived neurotrophic factor (BDNF) as shown in SEQ ID NO: 30, the signal peptide of neurotrophin-3 (NTF-3) as shown in SEQ ID NO:102, the signal peptide of fibroblast growth factor 5 (FGF5) as shown in SEQ ID NO:87, the signal peptide of insulin-like growth factor-binding protein 5 (IBP5) as shown in SEQ ID NO:97, the signal peptide of prostate and testis expressed protein 2 (PATE2) as shown in SEQ ID NO:107, the signal peptide of extracellular superoxide dismutase (SOD3) as shown in SEQ ID NO:112, and the signal peptide of complement factor H-related protein 2 (FHR2) as shown in SEQ ID NO:92.

In one embodiment of the present invention the signal peptide is i) a signal peptide heterologous to said protein modified by insertion, deletion and/or substitution of at least one amino acid, wherein the signal peptide heterologous to said protein is selected from the group consisting of the signal peptide of C-X-C Motif Chemokine Ligand 12 (CXCL12), the signal peptide of insulin growth factor 2 (IGF2), the signal peptide of insulin (INS), and the signal peptide of brain-derived neurotrophic factor (BDNF).

In a preferred embodiment of the present invention the signal peptide heterologous to said protein modified by insertion, deletion and/or substitution of at least one amino acid is selected from the group consisting of the modified signal peptide of C-X-C Motif Chemokine Ligand 12 (CXCL12) as shown in SEQ ID NO:132, the modified signal peptide of insulin growth factor 2 (IGF2) as shown in SEQ ID NO:127, the modified signal peptide of insulin (INS) as shown in SEQ ID NO:147, and the modified signal peptide of brain-derived neurotrophic factor (BDNF) as shown in SEQ ID NO:137.

In one embodiment of the present invention the signal peptide is ii) a signal peptide homologous to said protein, wherein the signal peptide homologous to said protein is modified by insertion, deletion and/or substitution of at least one amino acid, wherein the signal peptide homologous to said protein and said protein are selected from the group consisting of the signal peptide of insulin growth factor 1 (IGF1) and IGF1, the signal peptide of insulin and INS, the signal peptide of erythropoietin (EPO) and EPO, the signal peptide of interleukin 4 (IL-4) and IL-4, and the signal peptide of interleukin 10 (IL-10) and IL-10.

In a preferred embodiment of the present invention the signal peptide homologous to said protein modified by insertion, deletion and/or substitution of at least one amino acid is selected from the group consisting of the modified signal peptide of insulin growth factor 1 (IGF1) as shown in SEQ ID NO:122, the modified signal peptide of insulin as shown in SEQ ID NO:147, the modified signal peptide of erythropoietin (EPO) as shown in SEQ ID NO:152, the modified signal peptide of interleukin 4 (IL-4) as shown in SEQ ID NO:166, and the modified signal peptide of interleukin 10 (IL-10) as shown in SEQ ID NO:174.

In one embodiment of the present invention the signal peptide is iii) a naturally occurring amino acid sequence which does not have the function of a signal peptide in nature, wherein the naturally occurring amino acid sequence is optionally modified by insertion, deletion and/or substitution of at least one amino acid, wherein the naturally occurring amino acid sequence is selected from the group consisting of the pro-peptide of insulin growth factor 1 (IGF1), the coding sequence of glucagon receptor (GL-R) and the pro-peptide of intestinal-type alkaline phosphatase (ALPI).

In one embodiment of the present invention the signal peptide is iii) a naturally occurring amino acid sequence which does not have the function of a signal peptide in nature, wherein the naturally occurring amino acid sequence is the coding sequence of glucagon receptor (GL-R), iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of insulin growth factor 1 (IGF1) or iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of intestinal-type alkaline phosphatase (ALPI).

In one embodiment of the present invention the signal peptide is iii) a naturally occurring amino acid sequence which does not have the function of a signal peptide in nature, wherein the naturally occurring amino acid sequence is the coding sequence of glucagon receptor (GL-R)

In one embodiment of the present invention the signal peptide is iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of insulin growth factor 1 (IGF1).

In one embodiment of the present invention the signal peptide is iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of intestinal-type alkaline phosphatase (ALPI).

In a preferred embodiment of the present invention the signal peptide is iii) a naturally occurring amino acid sequence which does not have the function of a signal peptide in nature, wherein the naturally occurring amino acid sequence is the coding sequence of glucagon receptor (GL-R) as shown in SEQ ID NO:117, iii) a naturally occurring amino acid sequence which does not have the function of a signal peptide in nature, wherein the naturally occurring amino acid sequence is modified by insertion, deletion and/or substitution of at least one amino acid, wherein the modified naturally occurring amino acid sequence is the modified pro-peptide of insulin growth factor 1 (IGF1) as shown in SEQ ID NO:142 or iii) a naturally occurring amino acid sequence which does not have the function of a signal peptide in nature, wherein the naturally occurring amino acid sequence is modified by insertion, deletion and/or substitution of at least one amino acid, wherein the modified naturally occurring amino acid sequence is the modified pro-peptide of intestinal-type alkaline phosphatase (ALPI) as shown in SEQ ID NO:189.

In a preferred embodiment of the present invention the signal peptide is iii) a naturally occurring amino acid sequence which does not have the function of a signal peptide in nature, wherein the naturally occurring amino acid sequence is the coding sequence of glucagon receptor (GL-R) as shown in SEQ ID NO:117.

In a preferred embodiment of the present invention the signal peptide is iii) a naturally occurring amino acid sequence which does not have the function of a signal peptide in nature, wherein the naturally occurring amino acid sequence is modified by insertion, deletion and/or substitution of at least one amino acid, wherein the modified naturally occurring amino acid sequence is the modified pro-peptide of insulin growth factor 1 (IGF1) as shown in SEQ ID NO:142.

In a preferred embodiment of the present invention the signal peptide is iii) a naturally occurring amino acid sequence which does not have the function of a signal peptide in nature, wherein the naturally occurring amino acid sequence is modified by insertion, deletion and/or substitution of at least one amino acid, wherein the modified naturally occurring amino acid sequence is the modified pro-peptide of intestinal-type alkaline phosphatase (ALPI) as shown in SEQ ID NO:189.

In one embodiment of the present invention the signal peptide is i) a signal peptide heterologous to said protein, wherein the signal peptide heterologous to said protein is optionally modified by insertion, deletion and/or substitution of at least one amino acid and wherein the quantity of the secreted protein using the signal peptide heterologous to said protein is higher than the quantity of said secreted protein using the signal peptide homologous to said protein. Preferably the quantity of the secreted protein using the signal peptide heterologous to said protein is higher than, preferably at least 1.4 times higher than the quantity, of said secreted protein using the signal peptide homologous to said protein.

In one embodiment of the present invention the signal peptide is ii) a signal peptide homologous to said protein, wherein the signal peptide homologous to said protein is modified by insertion, deletion and/or substitution of at least one amino acid and wherein the quantity of the secreted protein using the modified signal peptide homologous to said protein is higher than the quantity of said secreted protein using the signal peptide homologous to said protein without modification. Preferably the quantity of the secreted protein using the modified signal peptide homologous to said protein is higher than, preferably at least 1.4 times higher than the quantity, of said secreted protein using the unmodified signal peptide homologous to said protein.

In one embodiment of the present invention the signal peptide is iii) a naturally occurring amino acid sequence which does not have the function of a signal peptide in nature, wherein the naturally occurring amino acid sequence is optionally modified by insertion, deletion and/or substitution of at least one amino acid and wherein the quantity of the secreted protein using the naturally occurring amino acid sequence which does not have the function of a signal peptide in nature is higher than the quantity of said secreted protein using the signal peptide homologous to said protein. Preferably the quantity of the secreted protein using the optionally modified naturally occurring amino acid sequence is higher than, preferably at least 1.4 times higher than the quantity of said secreted protein using the signal peptide homologous to said protein.

In one embodiment of the present invention wherein the signal peptide is i) a signal peptide heterologous to said protein, wherein the signal peptide heterologous to said protein is optionally modified by insertion, deletion and/or substitution of at least one amino acid, with the proviso that said protein is not a thioredoxin, more particular wherein the protein is not rod-derived cone viability factor.

In one embodiment of the present invention the signal peptide is selected from

i) a signal peptide heterologous to said protein and the protein is selected from the group consisting of insulin growth factor 1 (IGF1), insulin (INS), erythropoietin (EPO), interleukin-4 (IL-4) and interleukin-10 (IL-10).
i) a signal peptide heterologous to said protein, wherein the signal peptide heterologous to said protein is modified by insertion, deletion and/or substitution of at least one amino acid and the protein is IGF1.
ii) a signal peptide homologous to said protein, wherein the signal peptide homologous to said protein is modified by insertion, deletion and/or substitution of at least one amino acid and the protein is selected from the group consisting of insulin growth factor 1 (IGF1), insulin (INS), erythropoietin (EPO), interleukin-4 (IL-4) and interleukin-10 (IL-10);
iii) a naturally occurring amino acid sequence which does not have the function of a signal peptide in nature, wherein the naturally occurring amino acid sequence is the coding sequence of glucagon receptor (GL-R) and the protein is insulin growth factor 1 (IGF1); and
iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of insulin growth factor 1 (IGF1) and the protein is insulin growth factor 1 (IGF1).

In one embodiment of the present invention the signal peptide is i) a signal peptide heterologous to said protein and the protein is selected from the group consisting of insulin growth factor 1 (IGF1), insulin (INS), erythropoietin (EPO), interleukin-4 (IL-4) and interleukin-10 (IL-10).

In a preferred embodiment of the present invention the signal peptide is i) a signal peptide heterologous to said protein and the protein is selected from the group consisting of insulin growth factor 1 (IGF1) as shown in SEQ ID NO:188, insulin (INS) as shown in SEQ ID NO:185, erythropoietin (EPO) as shown in SEQ ID NO:184, interleukin-4 (IL-4) as shown in SEQ ID NO:186 and interleukin-10 (IL-10) as shown in SEQ ID NO:187.

In one embodiment of the present invention the signal peptide is i) a signal peptide heterologous to said protein, wherein the signal peptide heterologous to said protein is modified by insertion, deletion and/or substitution of at least one amino acid and the protein is IGF1.

In a preferred embodiment of the present invention the signal peptide is i) a signal peptide heterologous to said protein, wherein the signal peptide heterologous to said protein is modified by insertion, deletion and/or substitution of at least one amino acid and the protein is IGF1 as shown in SEQ ID NO:188.

In one embodiment of the present invention the signal peptide is ii) a signal peptide homologous to said protein, wherein the signal peptide homologous to said protein is modified by insertion, deletion and/or substitution of at least one amino acid and the protein is selected from the group consisting of insulin growth factor 1 (IGF1), insulin (INS), erythropoietin (EPO), interleukin-4 (IL-4) and interleukin-10 (IL-10).

In a preferred embodiment of the present invention the signal peptide is ii) a signal peptide homologous to said protein, wherein the signal peptide homologous to said protein is modified by insertion, deletion and/or substitution of at least one amino acid and the protein is selected from the group consisting of insulin growth factor 1 (IGF1) as shown in SEQ ID NO:188 insulin (INS) as shown in SEQ ID NO:185, erythropoietin (EPO) as shown in SEQ ID NO:184, interleukin-4 (IL-4) as shown in SEQ ID NO:186 and interleukin-10 (IL-10) as shown in SEQ ID NO: 187.

In one embodiment of the present invention the signal peptide is iii) a naturally occurring amino acid sequence which does not have the function of a signal peptide in nature, wherein the naturally occurring amino acid sequence is the coding sequence of glucagon receptor (GL-R) and the protein is insulin growth factor 1 (IGF1).

In one embodiment of the present invention the signal peptide is iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of insulin growth factor 1 (IGF1) and the protein is insulin growth factor 1 (IGF1).

In one embodiment of the present invention the signal peptide is iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of intestinal-type alkaline phosphatase (ALPI) and the protein is insulin growth factor 1 (IGF1).

In one embodiment of the present invention the signal peptide is iii) a naturally occurring amino acid sequence which does not have the function of a signal peptide in nature, wherein the naturally occurring amino acid sequence is the coding sequence of glucagon receptor (GL-R) and the protein is insulin growth factor 1 (IGF1) as shown in SEQ ID NO:188.

In one embodiment of the present invention the signal peptide is iii) a naturally occurring amino acid sequence which does not have the function of a signal peptide in nature, wherein the naturally occurring amino acid sequence is modified by insertion, deletion and/or substitution of at least one amino acid, wherein the modified naturally occurring amino acid sequence is the modified pro-peptide of insulin growth factor 1 (IGF1) and the protein is insulin growth factor 1 (IGF1) as shown in SEQ ID NO:188.

In one embodiment of the present invention the signal peptide is iii) a naturally occurring amino acid sequence which does not have the function of a signal peptide in nature, wherein the naturally occurring amino acid sequence is modified by insertion, deletion and/or substitution of at least one amino acid, wherein the modified naturally occurring amino acid sequence is the modified pro-peptide of intestinal-type alkaline phosphatase (ALPI) and the protein is insulin growth factor 1 (IGF1) as shown in SEQ ID NO:188.

In a preferred embodiment of the present invention the signal peptide is selected from the group consisting of

i) a signal peptide heterologous to said protein, wherein the signal peptide heterologous to said protein is selected from the group consisting of the signal peptide of brain-derived neurotrophic factor (BDNF), the signal peptide of neurotrophin-3 (NTF-3), the signal peptide of fibroblast growth factor 5 (FGF5), the signal peptide of insulin-like growth factor-binding protein 5 (MPS), the signal peptide of prostate and testis expressed protein 2 (PATE2), the signal peptide of extracellular superoxide dismutase (SOD3), and the signal peptide of complement factor H-related protein 2 (FHR2), with the proviso that said protein is not an oxidoreductase;

i) a signal peptide heterologous to said protein, wherein the signal peptide heterologous to said protein is modified by insertion, deletion and/or substitution of at least one amino acid, wherein the signal peptide heterologous to said protein is selected from the group consisting of the signal peptide of C-X-C Motif Chemokine Ligand 12 (CXCL12), the signal peptide of insulin growth factor 2 (IGF2), the signal peptide of insulin (INS), and the signal peptide of brain-derived neurotrophic factor (BDNF), with the proviso that said protein is not an oxidoreductase;

ii) a signal peptide homologous to said protein, wherein the signal peptide homologous to said protein is modified by insertion, deletion and/or substitution of at least one amino acid, wherein the signal peptide homologous to said protein is selected from the group consisting of the signal peptide of insulin growth factor 1 (IGF1), the signal peptide of insulin (INS), the signal peptide of erythropoietin (EPO), the signal peptide of interleukin 4 (IL-4), and the signal peptide of interleukin 10 (IL-10);
iii) a naturally occurring amino acid sequence which does not have the function of a signal peptide in nature, wherein the naturally occurring amino acid sequence is the coding sequence of glucagon receptor (GL-R);
iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of insulin growth factor 1 (IGF1); and
iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of intestinal-type alkaline phosphatase (ALPI).

In a preferred embodiment of the present invention the signal peptide is selected from the group consisting of

i) a signal peptide heterologous to said protein, wherein the signal peptide heterologous to said protein is selected from the group consisting of the signal peptide of brain-derived neurotrophic factor (BDNF), the signal peptide of neurotrophin-3 (NTF-3), the signal peptide of fibroblast growth factor 5 (FGF5), the signal peptide of insulin-like growth factor-binding protein 5 (IBP5), the signal peptide of prostate and testis expressed protein 2 (PATE2), the signal peptide of extracellular superoxide dismutase (SOD3), and the signal peptide of complement factor H-related protein 2 (FHR2) and said protein is selected from the group consisting of cytokines, growth factors and hormones;
i) a signal peptide heterologous to said protein, wherein the signal peptide heterologous to said protein is modified by insertion, deletion and/or substitution of at least one amino acid, wherein the signal peptide heterologous to said protein is selected from the group consisting of consisting of the signal peptide of C-X-C Motif Chemokine Ligand 12 (CXCL12), the signal peptide of insulin growth factor 2 (IGF2), the signal peptide of insulin (INS), and the signal peptide of brain-derived neurotrophic factor (BDNF) and said protein is selected from the group consisting of cytokines, growth factors and hormones;
ii) a signal peptide homologous to said protein, wherein the signal peptide homologous to said protein is modified by insertion, deletion and/or substitution of at least one amino acid, wherein the signal peptide homologous to said protein and said protein are selected from the group consisting of the signal peptide of insulin growth factor 1 (IGF1) and IGF1, the signal peptide of insulin and INS, the signal peptide of erythropoietin (EPO) and EPO, the signal peptide of interleukin 4 (IL-4) and IL-4, the signal peptide of interleukin 10 (IL-10) and IL-10;
iii) a naturally occurring amino acid sequence which does not have the function of a signal peptide in nature, wherein the signal peptide is the coding sequence of glucagon receptor (GL-R) and said protein is selected from the group consisting of cytokines; growth factors; and hormones, and is preferably a growth factor;
iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of insulin growth factor 1 (IGF1) and said protein is selected from the group consisting of cytokines, growth factors and hormones, and is preferably a growth factor; and
iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of intestinal-type alkaline phosphatase (ALPI) and said protein is selected from the group consisting of cytokines, growth factors and hormones, and is preferably a growth factor.

In a preferred embodiment of the present invention the signal peptide is selected from the group consisting of

i) a signal peptide heterologous to said protein, wherein the signal peptide heterologous to said protein is selected from the group consisting of the signal peptide of brain-derived neurotrophic factor (BDNF), the signal peptide of neurotrophin-3 (NTF-3), the signal peptide of fibroblast growth factor 5 (FGF5), the signal peptide of insulin-like growth factor-binding protein 5 (IBP5), the signal peptide of prostate and testis expressed protein 2 (PATE2), the signal peptide of extracellular superoxide dismutase (SOD3), and the signal peptide of complement factor H-related protein 2 (FHR2) and said protein is selected from the group consisting of insulin growth factor 1 (IGF1), insulin (INS), erythropoietin (EPO), interleukin 4 (IL-4), and interleukin 10 (IL-10);
i) a signal peptide heterologous to said protein, wherein the signal peptide heterologous to said protein is modified by insertion, deletion and/or substitution of at least one amino acid, wherein the signal peptide heterologous to said protein is selected from the group consisting of consisting of the signal peptide of C-X-C Motif Chemokine Ligand 12 (CXCL12), the signal peptide of insulin growth factor 2 (IGF2), the signal peptide of insulin (INS), and the signal peptide of brain-derived neurotrophic factor (BDNF) and said protein is insulin growth factor 1 (IGF1);
ii) a signal peptide homologous to said protein, wherein the signal peptide homologous to said protein is modified by insertion, deletion and/or substitution of at least one amino acid, wherein the signal peptide homologous to said protein and said protein are selected from the group consisting of the signal peptide of insulin growth factor 1 (IGF1) and IGF1, the signal peptide of insulin and INS, the signal peptide of erythropoietin (EPO) and EPO, the signal peptide of interleukin 4 (IL-4) and IL-4, the signal peptide of interleukin 10 (IL-10) and IL-10;
iii) a naturally occurring amino acid sequence which does not have the function of a signal peptide in nature, wherein the naturally occurring amino acid sequence is the coding sequence of glucagon receptor (GL-R) and said protein is insulin growth factor 1 (IGF1);
iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of insulin growth factor 1 (IGF1) and said protein is insulin growth factor 1 (IGF1); and
iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of intestinal-type alkaline phosphatase (ALPI) and said protein is insulin growth factor 1 (IGF1).

In a preferred embodiment of the present invention the signal peptide is selected from the group consisting of

i) a signal peptide heterologous to said protein, wherein the signal peptide heterologous to said protein and said protein are selected from the group consisting of the signal peptide of brain-derived neurotrophic factor (BDNF) and IGF1, insulin, EPO, or IL-10, the signal peptide of neurotrophin-3 (NTF-3) and IGF1, the signal peptide of fibroblast growth factor 5 (FGF5) and IGF1 or IL4, the signal peptide of insulin-like growth factor-binding protein 5 (MPS) and IGF1, the signal peptide of prostate and testis expressed protein 2 (PATE2) and IGF1, the signal peptide of extracellular superoxide dismutase (SOD3) and IGF1, and the signal peptide of complement factor H-related protein 2 (FHR2) and IGF1;
i) a signal peptide heterologous to said protein, wherein the signal peptide heterologous to said protein is modified by insertion, deletion and/or substitution of at least one amino acid, wherein the signal peptide heterologous to said protein and said protein are selected from the group consisting of the signal peptide of C-X-C Motif Chemokine Ligand 12 (CXCL12) and IGF1, the signal peptide of insulin growth factor 2 (IGF2) and IGF1, the signal peptide of insulin (INS) and IGF1, and the signal peptide of brain-derived neurotrophic factor (BDNF) and IGF1;
ii) a signal peptide homologous to said protein, wherein the signal peptide homologous to said protein is modified by insertion, deletion and/or substitution of at least one amino acid, wherein the signal peptide homologous to said protein and the protein are selected from the group consisting of the signal peptide of insulin growth factor 1 (IGF1) and IGF1, the signal peptide of insulin and INS, the signal peptide of erythropoietin (EPO) and EPO, the signal peptide of interleukin 4 (IL-4) and IL-4, the signal peptide of interleukin 10 (IL-10) and IL-10;
iii) a naturally occurring amino acid sequence which does not have the function of a signal peptide in nature, wherein the naturally occurring amino acid sequence is the coding sequence of glucagon receptor (GL-R) and said protein is insulin growth factor 1 (IGF1);
iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of insulin growth factor 1 (IGF1) and said protein is insulin growth factor 1 (IGF1); and
iii) a naturally occurring amino acid sequence which does not have the function of a signal peptide in nature, wherein the naturally occurring amino acid sequence is modified by insertion, deletion and/or substitution of at least one amino acid, wherein the naturally occurring amino acid sequence is the pro-peptide of intestinal-type alkaline phosphatase (ALPI) and said protein is insulin growth factor 1 (IGF1).

In a preferred embodiment of the present invention the signal peptide is selected from the group consisting of

i) a signal peptide heterologous to said protein, wherein the signal peptide heterologous to said protein is selected from the group consisting of the signal peptide of brain-derived neurotrophic factor (BDNF) as shown in SEQ ID NO:30, the signal peptide of neurotrophin-3 (NTF-3) as shown in SEQ ID NO:102, the signal peptide of fibroblast growth factor 5 (FGF5) as shown in SEQ ID NO:87, the signal peptide of insulin-like growth factor-binding protein 5 (IBP5) as shown in SEQ ID NO:97, the signal peptide of prostate and testis expressed protein 2 (PATE2) as shown in SEQ ID NO:107, the signal peptide of extracellular superoxide dismutase (SOD3) as shown in SEQ ID NO:112, and the signal peptide of complement factor H-related protein 2 (FHR2) as shown in SEQ ID NO:92, with the proviso that said protein is not an oxidoreductase;
i) a signal peptide heterologous to said protein, wherein the signal peptide heterologous to said protein is modified by insertion, deletion and/or substitution of at least one amino acid, wherein the signal peptide heterologous to said protein is selected from the group consisting of the signal peptide of C-X-C Motif Chemokine Ligand 12 (CXCL12) as shown in SEQ ID NO:132, the signal peptide of insulin growth factor 2 (IGF2) as shown in SEQ ID NO:127, the signal peptide of insulin (INS) as shown in SEQ ID NO:147, and the signal peptide of brain-derived neurotrophic factor (BDNF) as shown in SEQ ID NO:137, with the proviso that said protein is not an oxidoreductase;
ii) a signal peptide homologous to said protein, wherein the signal peptide homologous to said protein is modified by insertion, deletion and/or substitution of at least one amino acid, wherein the signal peptide homologous to said protein is selected from the group consisting of the modified signal peptide of insulin growth factor 1 (IGF1) as shown in SEQ ID NO:122, the modified signal peptide of insulin (INS) as shown in SEQ ID NO:147, the modified signal peptide of erythropoietin (EPO) as shown in SEQ ID NO:152, the modified signal peptide of interleukin 4 (IL-4) as shown in SEQ ID NO:166, and the modified signal peptide of interleukin 10 (IL-10) as shown in SEQ ID NO:174;
iii) a naturally occurring amino acid sequence which does not have the function of a signal peptide in nature, wherein the naturally occurring amino acid sequence is the coding sequence of glucagon receptor (GL-R) as shown in SEQ ID NO:117;
iii) a naturally occurring amino acid sequence which does not have the function of a signal peptide in nature, wherein the naturally occurring amino acid sequence is modified by insertion, deletion and/or substitution of at least one amino acid, wherein the modified naturally occurring amino acid sequence is the modified pro-peptide of insulin growth factor 1 (IGF1) as shown in SEQ ID NO:142; and
iii) a naturally occurring amino acid sequence which does not have the function of a signal peptide in nature, wherein the naturally occurring amino acid sequence is modified by insertion, deletion and/or substitution of at least one amino acid, wherein the modified naturally occurring amino acid sequence is the modified pro-peptide of intestinal-type alkaline phosphatase (ALPI) as shown in SEQ ID NO:189.

In a preferred embodiment of the present invention the signal peptide is selected from the group consisting of

i) a signal peptide heterologous to said protein, wherein the signal peptide heterologous to said protein is selected from the group consisting of the signal peptide of brain-derived neurotrophic factor (BDNF) as shown in SEQ ID NO:30, the signal peptide of neurotrophin-3 (NTF-3) as shown in SEQ ID NO:102, the signal peptide of fibroblast growth factor 5 (FGF5) as shown in SEQ ID NO:87, the signal peptide of insulin-like growth factor-binding protein 5 (IBP5) as shown in SEQ ID NO:97, the signal peptide of prostate and testis expressed protein 2 (PATE2) as shown in SEQ ID NO:107, the signal peptide of extracellular superoxide dismutase (SOD3) as shown in SEQ ID NO:112, and the signal peptide of complement factor H-related protein 2 (FHR2) as shown in SEQ ID NO:92 and said protein is selected from the group consisting of cytokines, growth factors and hormones;
i) a signal peptide heterologous to said protein, wherein the signal peptide heterologous to said protein is modified by insertion, deletion and/or substitution of at least one amino acid, wherein the modified signal peptide heterologous to said protein is selected from the group consisting of the modified signal peptide of C-X-C Motif Chemokine Ligand 12 (CXCL12) as shown in SEQ ID NO:132, the modified signal peptide of insulin growth factor 2 (IGF2) as shown in SEQ ID NO:127, the modified signal peptide of insulin (INS) as shown in SEQ ID NO:147, and the modified signal peptide of brain-derived neurotrophic factor (BDNF) as shown in SEQ ID NO:137 and said protein is selected from the group consisting of cytokines growth factors and hormones;
ii) a signal peptide homologous to said protein, wherein the signal peptide homologous to said protein is modified by insertion, deletion and/or substitution of at least one amino acid, wherein the modified signal peptide homologous to said protein and said protein are selected from the group consisting of the modified signal peptide of insulin growth factor 1 (IGF1) as shown in SEQ ID NO:122 and IGF1 as shown in SEQ ID NO:188, the modified signal peptide of insulin as shown in SEQ ID NO:147 and insulin (INS) as shown in SEQ ID NO:185, the modified signal peptide of erythropoietin (EPO) as shown in SEQ ID NO:152 and EPO as shown in SEQ ID NO:184, the modified signal peptide of interleukin 4 (IL-4) as shown in SEQ ID NO:166 and IL-4 as shown in SEQ ID NO:186, the modified signal peptide of interleukin 10 (IL-10) as shown in SEQ ID NO:174 and IL-10 as shown in SEQ ID NO:187;
iii) a naturally occurring amino acid sequence which does not have the function of a signal peptide in nature, wherein the naturally occurring amino acid sequence is the coding sequence of glucagon receptor (GL-R) as shown in SEQ ID NO:117 and said protein is selected from the group consisting of cytokines; growth factors; and hormones, and is preferably a growth factor;
iii) a naturally occurring amino acid sequence which does not have the function of a signal peptide in nature, wherein the naturally occurring amino acid sequence is modified by insertion, deletion and/or substitution of at least one amino acid, wherein the modified naturally occurring amino acid sequence is the modified pro-peptide of insulin growth factor 1 (IGF1) as shown in SEQ ID NO:142 and said protein is selected from the group consisting of cytokines; growth factors; and hormones, and is preferably a growth factor; and
iii) a naturally occurring amino acid sequence which does not have the function of a signal peptide in nature, wherein the naturally occurring amino acid sequence is modified by insertion, deletion and/or substitution of at least one amino acid, wherein the modified naturally occurring amino acid sequence is the modified pro-peptide of intestinal-type alkaline phosphatase (ALPI) as shown in SEQ ID NO:189 and said protein is selected from the group consisting of cytokines; growth factors; and hormones, and is preferably a growth factor.

In a preferred embodiment of the present invention the signal peptide is selected from the group consisting of

i) a signal peptide heterologous to said protein, wherein the signal peptide heterologous to said protein is selected from the group consisting of the signal peptide of brain-derived neurotrophic factor (BDNF) as shown in SEQ ID NO:30, the signal peptide of neurotrophin-3 (NTF-3) as shown in SEQ ID NO:102, the signal peptide of fibroblast growth factor 5 (FGF5) as shown in SEQ ID NO:87, the signal peptide of insulin-like growth factor-binding protein 5 (IBP5) as shown in SEQ ID NO:97, the signal peptide of prostate and testis expressed protein 2 (PATE2) as shown in SEQ ID NO:107, the signal peptide of extracellular superoxide dismutase (SOD3) as shown in SEQ ID NO:112, and the signal peptide of complement factor H-related protein 2 (FHR2) as shown in SEQ ID NO:92 and said protein is selected from the group consisting of insulin growth factor 1 (IGF1) as shown in SEQ ID NO:188, insulin as shown in SEQ ID NO:185, erythropoietin (EPO) as shown in SEQ ID NO:184, interleukin 4 (IL-4) as shown in SEQ ID NO:186, and interleukin 10 (IL-10) as shown in SEQ ID NO:187;
i) a signal peptide heterologous to said protein, wherein the signal peptide heterologous to said protein is modified by insertion, deletion and/or substitution of at least one amino acid, wherein the modified signal peptide heterologous to said protein is selected from the group consisting of the modified signal peptide of C-X-C Motif Chemokine Ligand 12 (CXCL12) as shown in SEQ ID NO:132, the modified signal peptide of insulin growth factor 2 (IGF2) as shown in SEQ ID NO:127, the modified signal peptide of insulin (INS) as shown in SEQ ID NO:147, and the modified signal peptide of brain-derived neurotrophic factor (BDNF) as shown in SEQ ID NO:137 and said protein is insulin growth factor 1 (IGF1) as shown in SEQ ID NO:188;
ii) a signal peptide homologous to said protein, wherein the signal peptide homologous to said protein is modified by insertion, deletion and/or substitution of at least one amino acid, wherein the modified signal peptide homologous to said protein and said protein are selected from the group consisting of the modified signal peptide of insulin growth factor 1 (IGF1) as shown in SEQ ID NO:122 and IGF1 as shown in SEQ ID NO:188, the modified signal peptide of insulin as shown in SEQ ID NO:147 and insulin (INS) as shown in SEQ ID NO:185, the modified signal peptide of erythropoietin (EPO) as shown in SEQ ID NO:152 and EPO as shown in SEQ ID NO:184, the modified signal peptide of interleukin 4 (IL-4) as shown in SEQ ID NO:166 and IL-4 as shown in SEQ ID NO:186, the modified signal peptide of interleukin 10 (IL-10) as shown in SEQ ID NO:174 and IL-10 as shown in SEQ ID NO:187;
iii) a naturally occurring amino acid sequence which does not have the function of a signal peptide in nature, wherein the naturally occurring amino acid sequence is the coding sequence of glucagon receptor (GL-R) as shown in SEQ ID NO:117 and said protein is insulin growth factor 1 (IGF1) as shown in SEQ ID NO:188;
iii) a naturally occurring amino acid sequence which does not have the function of a signal peptide in nature, wherein the naturally occurring amino acid sequence is modified by insertion, deletion and/or substitution of at least one amino acid, wherein the modified naturally occurring amino acid sequence is the modified pro-peptide of insulin growth factor 1 (IGF1) as shown in SEQ ID NO:142 and said protein is insulin growth factor 1 (IGF1) as shown in SEQ ID NO:188, and
iii) a naturally occurring amino acid sequence which does not have the function of a signal peptide in nature, wherein the naturally occurring amino acid sequence is modified by insertion, deletion and/or substitution of at least one amino acid, wherein the modified naturally occurring amino acid sequence is the modified pro-peptide of intestinal-type alkaline phosphatase (ALPI) as shown in SEQ ID NO:189 and said protein is insulin growth factor 1 (IGF1) as shown in SEQ ID NO:188.

In a particular preferred embodiment of the present invention the mRNA comprising a nucleic acid sequence encoding a protein and a signal peptide is selected from the group consisting of the mRNA sequence as shown in SEQ ID NO:8, the mRNA sequence as shown in SEQ ID NO:105, the mRNA sequence as shown in SEQ ID NO:90, the mRNA sequence as shown in SEQ ID NO:100, the mRNA sequence as shown in SEQ ID NO:110, the mRNA sequence as shown in SEQ ID NO:115, the mRNA sequence as shown in SEQ ID NO:95, the mRNA sequence as shown in SEQ ID NO:135, the mRNA sequence as shown in SEQ ID NO:130, the mRNA sequence as shown in SEQ ID NO:150, the mRNA sequence as shown in SEQ ID NO:140, the mRNA sequence as shown in SEQ ID NO:125, the mRNA sequence as shown in SEQ ID NO:161, the mRNA sequence as shown in SEQ ID NO:155, the mRNA sequence as shown in SEQ ID NO:169, the mRNA sequence as shown in SEQ ID NO:177, the mRNA sequence as shown in SEQ ID NO:120, the mRNA sequence as shown in SEQ ID NO:145, and the mRNA sequence as shown in SEQ ID NO:192.

In a further aspect the present invention provides a mRNA comprising a nucleic acid sequence encoding

i) a protein; and
ii) a signal peptide heterologous to said protein,
wherein the signal peptide heterologous to said protein is the signal peptide of the brain-derived neurotrophic factor (BDNF) and wherein the protein is not an oxidoreductase, in particular a mRNA comprising a nucleic acid sequence encoding
i) a protein; and
ii) a signal peptide heterologous to said protein,
wherein the signal peptide heterologous to said protein is the signal peptide of the brain-derived neurotrophic factor (BDNF) and wherein the protein is selected from the group consisting of carboxypeptidases; cytokines; extracellular ligands and transporters; extracellular matrix proteins; glucosidases; glycosyltransferases; growth factors; growth factor binding proteins; heparin binding proteins; hormones; hydrolases; immunoglobulins; isomerases; kinases; lyases; metalloenzyme inhibitors; metalloproteases; milk proteins; neuroactive proteins; proteases; protease inhibitors; protein phosphatases; esterases; transferases; and vasoactive proteins.

In one embodiment of the present invention the protein is a therapeutic protein. In a preferred embodiment of the present invention the protein is of human origin i.e. is a human protein. In a further preferred embodiment of the present invention the protein is selected from the group consisting of carboxypeptidases; cytokines; extracellular ligands and transporters; extracellular matrix proteins; glucosidases; glycosyltransferases; growth factors; growth factor binding proteins; heparin binding proteins; hormones; hydrolases; immunoglobulins; isomerases; kinases; lyases; metalloenzyme inhibitors; metalloproteases; milk proteins; neuroactive proteins; proteases; protease inhibitors; protein phosphatases; esterases; transferases; and vasoactive proteins all of human origin. In a more preferred embodiment of the present invention the protein of the present invention is a human protein selected from the group consisting of human carboxypeptidases; human cytokines; human extracellular ligands and transporters; human extracellular matrix proteins; human glucosidases; human glycosyltransferases; human growth factors; human growth factor binding proteins; human heparin binding proteins; human hormones; human hydrolases; human immunoglobulins; human isomerases; human kinases; human lyases; human metalloenzyme inhibitors; human metalloproteases; human milk proteins; human neuroactive proteins; human proteases; human protease inhibitors; human protein phosphatases; human esterases; human transferases; and human vasoactive proteins.

In one embodiment, the protein is selected from the group consisting of carboxypeptidases, wherein the carboxypeptidases are selected from the group consisting of ACE, ACE2, CNDP1, CPA1, CPA2, CPA4, CPA5, CPA6, CPB1, CPB2, CPE, CPN1, CPQ, CPXM1, CPZ, and SCPEP1; cytokines wherein the cytokines are selected from the group consisting of BMP1, BMP10, BMP15, BMP2, BMP3, BMP4, BMP5, BMP6, BMP7, BMP8A, BMP8B, C1QTNF4, CCL1, CCL11, CCL13, CCL14, CCL15, CCL16, CCL17, CCL18, CCL19, CCL2, CCL21, CCL22, CCL23, CCL24, CCL25, CCL26, CCL27, CCL28, CCL3, CCL3L1, CCL3L3, CCL4, CCL4L, CCL4L2, CCL5, CCL7, CCL8, CD40LG, CER1, CKLF, CLCF1, CNTF, CSF1, CSF2, CSF3, CTF1, CX3CL1, CXCL1, CXCL10, CXCL11, CXCL12, CXCL13, CXCL14, CXCL16, CXCL17, CXCL2, CXCL3, CXCL5, CXCL8, CXCL9, DKK1, DKK2, DKK3, DKK4, EDA, EBI3, FAM3B, FAM3C, FASLG, FLT3LG, GDF1, GDF10, GDF11, GDF15, GDF2, GDF3, GDF5, GDF6, GDF7, GDF9, GPI, GREM1, GREM2, GRN, IFNA1, IFNA13, IFNA10, IFNA14, IFNA16, IFNA17, IFNA2, IFNA21, IFNA4, IFNA5, IFNA6, IFNA7, IFNA8, IFNB1, IFNE, IFNG, IFNK, IFNL1, IFNL2, IFNL3, IFNL4, IFNW1, IL10, IL11, IL12A, IL12B, IL13, IL15, IL16, IL17A, IL17B, IL17C, IL17D, IL17F, IL18, IL19, IL1A, IL1B, IL1F10, IL2, IL20, IL21, IL22, IL23A, IL24, IL25, IL26, IL27, IL3, IL31, IL32, IL33, IL34, IL36A, IL36B, IL36G, IL36RN, IL37, IL4, IL5, IL6, IL7, IL9, LEFTY1, LEFTY2, LIF, LTA, MIF, MSTN, NAMPT, NODAL, OSM, PF4, PF4V1, SCGB3A1, SECTM1, SLURP1, SPP1, THNSL2, THPO, TNF, TNFSF10, TNFSF11, TNFSF12, TNFSF13, TNFSF13B, TNFSF14, TNFSF15, TSLP, VSTM1, WNT1, WNT10A, WNT10B, WNT11, WNT16, WNT2, WNT2B, WNT3, WNT3A, WNT4, WNT5A, WNT5B, WNT6, WNT7A, WNT7B, WNT8A, WNT8B, WNT9A, WNT9B, XCL1, and XCL2; extracellular ligands and transporters, wherein the extracellular ligands and transporters are selected from the group consisting of APCS, CHI3L1, CHI3L2, CLEC3B, DMBT1, DMKN, EDDM3A, EDDM3B, EFNA4, EMC10, ENAM, EPYC, ERVH48-1, F13B, FCN1, FCN2, GLDN, GPLD1, HEG1, ITFG1, KAZALD1, KCP, LACRT, LEG1, METRN, NOTCH2NL, NPNT, OLFM1, OLFML3, PRB2, PSAP, PSAPL1, PSG1, PSG6, PSG9, PTX3, PTX4, RBP4, RNASE10, RNASE12, RNASE13, RNASE9, RSPRY1, RTBDN, S100A12, S100A13, S100A7, S100A8, SAA2, SAA4, SCG1, SCG2, SCG3, SCGB1C1, SCGB1C2, SCGB1D1, SCGB1D2, SCGB1D4, SCGB2B2, SCGB3A2, SCGN, SCRG1, SCUBE1, SCUBE2, SCUBE3, SDCBP, SELENOP, SFTA2, SFTA3, SFTPA1, SFTPA2, SFTPC, SFTPD, SHBG, SLURP2, SMOC1, SMOC2, SMR3A, SMR3B, SNCA, SPATA20, SPATA6, SOGA1, SPARC, SPARCL1, SPATA20, SPATA6, SRPX2, SSC4D, STX1A, SUSD4, SVBP, TCN1, TCN2, TCTN1, TF, TULP3, TFF2, TFF3, THSD7A, TINAG, TINAGL1, TMEFF2, TMEM25, and VWC2L; extracellular matrix proteins, wherein the extracellular matrix proteins are selected from the group consisting of ABI3BP, AGRN, CCBE1, CHL1, COL15A1, COL19A1, COLEC11, DMBT1, DRAXIN, EDIL3, ELN, EMID1, EMILIN1, EMILIN2, EMILIN3, EPDR1, FBLN1, FBLN2, FBLNS, FLRT1, FLRT2, FLRT3, FREM1, GLDN, IB SP, KERA, KIAA0100, KIRREL3, KRT10, LAMB2, MGP, RPTN, SBSPON, SDC1, SDC4, SEMA3A, SEMA3B, SEMA3C, SEMA3D, SEMA3E, SEMA3F, SEMA3G, SIGLEC1, SIGLEC10, SIGLEC6, SLIT1, SLIT2, SLIT3, SLITRK1, SNED1, SNORC, SPACA3, SPACA7, SPON1, SPON2, STATH, SVEP1, TECTA, TECTB, TNC, TNN, TNR, and TNXB; glucosidases, wherein the glucosidases are selected from the group consisting of AMY1A, AMY1B, AMY1C, AMY2A, AMY2B, CEMIP, CHIA, CHIT1, FUCA2, GLB1L, GLB1L2, HPSE, HYAL1, HYAL3, KL, LYG1, LYG2, LYZL1, LYZL2, MAN2B2, SMPD1, SMPDL3B, SPACAS, and SPACASB; glycosyltransferases, wherein the glycosyltransferases are selected from the group consisting of ARTS, B4GALT1, EXTL2, GALNT1, GALNT2, GLT1D1, MGAT4A, ST3GAL1, ST3GAL2, ST3GAL3, ST3GAL4, ST6GAL1, and XYLT1; growth factors, wherein the growth factors are selected from the group consisting of AMH, ARTN, BTC, CDNF, CFC1, CFC1B, CHRDL1, CHRDL2, CLEC11A, CNMD, EFEMP1, EGF, EGFL6, EGFL7, EGFL8, EPGN, EREG, EYS, FGF1, FGF10, FGF16, FGF17, FGF18, FGF19, FGF2, FGF20, FGF21, FGF22, FGF23, FGF3, FGF4, FGF5, FGF6, FGF7, FGF8, FGF9, FRZB, GDNF, GFER, GKN1, HBEGF, HGF, IGF1, IGF2, INHA, INHBA, INHBB, INHBC, INHBE, INS, KITLG, MANF, MDK, MIA, NGF, NOV, NRG1, NRG2, NRG3, NRG4, NRTN, NTF3, NTF4, OGN, PDGFA, PDGFB, PDGFC, PDGFD, PGF, PROK1, PSPN, PTN, SDF1, SDF2, SFRP1, SFRP2, SFRP3, SFRP4, SFRP5, TDGF1, TFF1, TGFA, TGFB1, TGFB2, TGFB3, THBS4, TIMP1, VEGFA, VEGFB, VEGFC, VEGFD, and WISP3; growth factor binding proteins, wherein the growth factor binding proteins are selected from the group consisting of CHRD, CYR61, ESM1, FGFBP1, FGFBP2, FGFBP3, HTRA1, GHBP, IGFALS, IGFBP1, IGFBP2, IGFBP3, IGFBP4, IGFBP5, IGFBP6, IGFBP7, LTBP1, LTBP2, LTBP3, LTBP4, SOSTDC1, NOG, TWSG1, and WIF1; heparin binding proteins, wherein the heparin binding proteins are selected from the group consisting of ADA2, ADAMTSL5, ANGPTL3, APOB, APOE, APOH, COL5A1, COMP, CTGF, FBLN7, FN1, FSTL1, HRG, LAMC2, LIPC, LIPG, LIPH, LIPI, LPL, PCOLCE2, POSTN, RSPO1, RSPO2, RSPO3, RSPO4, SAA1, SLIT2, SOST, THBS1, and VTN; hormones, wherein the hormones are selected from the group consisting of ADCYAP1, ADIPOQ, ADM, ADM2, ANGPTL8, APELA, APLN, AVP, C1QTNF12, C1QTNF9, CALCA, CALCB, CCK, CGA, CGB1, CGB2, CGB3, CGBS, CGB8, COPA, CORT, CRH, CSH1, CSH2, CSHL1, ENHO, EPO, ERFE, FBN1, FNDCS, FSHB, GAL, GAST, GCG, GH, GH1, GH2, GHRH, GHRL, GIP, GNRH1, GNRH2, GPHA2, GPHB5, IAPP, INS, INSL3, INSL4, INSL5, INSL6, LHB, METRNL, MLN, NPPA, NPPB, NPPC, OSTN, OXT, PMCH, PPY, PRL, PRLH, PTH, PTHLH, PYY, REIN, RETNLB, RLN1, RLN2, RLN3, SCT, SPX, SST, STC1, STC2, TG, TOR2A, TRH, TSHB, TTR, UCN, UCN2, UCN3, UTS2, UTS2B, and VIP; hydrolases, wherein the hydrolases are selected from the group consisting of AADACL2, ABHD15, ACP7, ACPP, ADA2, ADAMTSL1, AOAH, ARSF, ARSI, ARSJ, ARSK, BTD, CHI3L2, ENPP1, ENPP2, ENPP3, ENPPS, ENTPDS, ENTPD6, GBP1, GGH, GPLD1, HPSE, LIPC, LIPF, LIPG, LIPH, LIPI, LIPK, LIPM, LIPN, LPL, PGLYRP2, PLA1A, PLA2G10, PLA2G12A, PLA2G1B, PLA2G2A, PLA2G2D, PLA2G2E, PLA2G2F, PLA2G3, PLA2G5, PLA2G7, PNLIP, PNLIPRP2, PNLIPRP3, PON1, PON3, PPT1, SMPDL3A, THEM6, THSD1, and THSD4; immunoglobulins, wherein the immunoglobulins are selected from the group consisting of IGSF10, IGKV1-12, IGKV1-16, IGKV1-33, IGKV1-6, IGKV1D-12, IGKV1D-39, IGKV1D-8, IGKV2-30, IGKV2D-30, IGKV3-11, IGKV3D-20, IGKV5-2, IGLC1, IGLC2, and IGLC3; isomerases, wherein the isomerases are selected from the group consisting of NAXE, PPIA, and PTGDS; kinases, wherein the kinases are selected from the group consisting of ADCK1, ADPGK, FAM20C, ICOS, and PKDCC; lyases, wherein the lyases are selected from the group consisting of PM20D1, PAM, and CA6; metalloenzyme inhibitors, wherein the metalloenzyme inhibitors are selected from the group consisting of FETUB, SPOCK3, TIMP2, TIMP3, TIMP4, WFIKKN1, and WFIKKN2; metalloproteases, wherein the metalloproteases are selected from the group consisting of ADAM12, ADAM28, ADAMS, ADAMDEC1, ADAMTS1, ADAMTS10, ADAMTS12, ADAMTS13, ADAMTS14, ADAMTS15, ADAMTS16, ADAMTS17, ADAMTS18, ADAMTS19, ADAMTS2, ADAMTS20, ADAMTS3, ADAMTS4, ADAMTS5, ADAMTS6, ADAMTS7, ADAMTS8, ADAMTS9, CLCA1, CLCA2, CLCA4, IDE, MEP1B, MMEL1, MMP1, MMP10, MMP11, MMP12, MMP13, MMP16, MMP17, MMP19, MMP2, MMP20, MMP21, MMP24, MMP25, MMP26, MMP28, MMP3, MMPI, MMP8, MMP9, PAPPA, PAPPA2, TLL1, and TLL2; milk proteins, wherein the milk proteins are selected from the group consisting of CSN1S1, CSN2, CSN3, and LALBA; neuroactive proteins, wherein the neuroactive proteins are selected from the group consisting of CARTPT, NMS, NMU, NPB, NPFF, NPS, NPVF, NPW, NPY, PCSK1N, PDYN, PENK, PNOC, POMC, PROK2, PTH2, PYY2, PYY3, QRFP, TAC1, and TAC3; proteases, wherein the proteases are selected from the group consisting of ADAMTS6, C1R, C1RL, C2, CASP4, CELA1, CELA2A, CELA2B, CFB, CFD, CFI, CMA1, CORIN, CTRB1, CTRB2, CTSB, CTSD, DHH, F10, F11, F12, F2, F3, F7, F8, F9, FAP, FURIN, GZMA, GZMK, GZMM, HABP2, HGFAC, HTRA3, HTRA4, IHH, KLK10, KLK11, KLK12, KLK13, KLK14, KLK15, KLK3, KLK4, KLK5, KLK6, KLK7, KLK8, KLK9, KLKB1, MASP1, MASP2, MST1L, NAPSA, OVCH1, OVCH2, PCSK2, PCSKS, PCSK6, PCSK9, PGA3, PGA4, PGA5, PGC, PLAT, PLAU, PLG, PROC, PRSS1, PRSS12, PRSS2, PRSS22, PRSS23, PRSS27, PRSS29P, PRSS3, PRSS33, PRSS36, PRSS38, PRSS3P2, PRSS42, PRSS44, PRSS47, PRSS48, PRSS53, PRSS57, PRSS58, PRSS8, PRTN3, RELN, REN, TMPRSS11D, TMPRSS11E, TMPRSS2, TPSAB1, TPSB2, and TPSD1; protease inhibitors, wherein the protease inhibitors are selected from the group consisting of A2M, A2ML1, AMBP, ANOS1, COL28A1, COL6A3, COL7A1, CPAMD8, CST1, CST2, CST3, CST4, CST5, CST6, CST7, CST8, CST9, CST9L, CST9LP1, CSTL1, EPPIN, GPC3, HMSD, ITIH1, ITIH2, ITIH3, ITIH4, ITIH5, ITIH6, KNG1, OPRPN, OVOS1, OVOS2, PAPLN, PI15, PI16, PI3, PZP, R3HDML, SERPINA1, SERPINA10, SERPINA11, SERPINA12, SERPINA13P, SERPINA3, SERPINA4, SERPINA5, SERPINA7, SERPINA9, SERPINB2, SERPINBS, SERPINC1, SERPINE1, SERPINE2, SERPINE3, SERPINF2, SERPING1, SERPINI1, SERPINI2, SPINK1, SPINK13, SPINK14, SPINK2, SPINK4, SPINK5, SPINK6, SPINK7, SPINK8, SPINK9, SPINT1, SPINT3, SPINT4, SPOCK1, SPOCK2, SPP2, SSPO, TFPI, TFPI2, WFDC1, WFDC10A, WFDC13, WFDC2, WFDC3, WFDC5, WFDC6, and WFDC8; protein phosphatases, wherein the protein phosphatases are selected from the group consisting of ACP7, ACPP, PTEN, and PTPRZ1; esterases, wherein the esterases, are selected from the group consisting of BCHE, CEL, CES4A, CES5A, NOTUM, and SIAE; transferases, wherein the transferases, are selected from the group consisting of METTL24, FKRP, CHSY1, CHST9, and B3GAT1; and vasoactive proteins, wherein the vasoactive proteins are selected from the group consisting of AGGF1, AGT, ANGPT1, ANGPT2, ANGPTL4, ANGPTL6, EDN1, EDN2, EDN3, and NTS.

In a preferred embodiment, the protein is selected from the group constisting of cytokines; growth factors; growth factor binding proteins; heparin binding proteins; hormones; neuroactive proteins; and vasoactive proteins.

In a more preferred embodiment, the protein is selected from the group constisting of cytokines wherein the cytokines are selected from the group consisting of BMP1, BMP10, BMP15, BMP2, BMP3, BMP4, BMP5, BMP6, BMP7, BMP8A, BMP8B, C1QTNF4, CCL1, CCL11, CCL13, CCL14, CCL15, CCL16, CCL17, CCL18, CCL19, CCL2, CCL21, CCL22, CCL23, CCL24, CCL25, CCL26, CCL27, CCL28, CCL3, CCL3L1, CCL3L3, CCL4, CCL4L, CCL4L2, CCL5, CCL7, CCL8, CD40LG, CER1, CKLF, CLCF1, CNTF, CSF1, CSF2, CSF3, CTF1, CX3CL1, CXCL1, CXCL10, CXCL11, CXCL12, CXCL13, CXCL14, CXCL16, CXCL17, CXCL2, CXCL3, CXCL5, CXCL8, CXCL9, DKK1, DKK2, DKK3, DKK4, EDA, EBI3, FAM3B, FAM3C, FASLG, FLT3LG, GDF1, GDF10, GDF11, GDF15, GDF2, GDF3, GDF5, GDF6, GDF7, GDF9, GPI, GREM1, GREM2, GRN, IFNA1, IFNA13, IFNA10, IFNA14, IFNA16, IFNA17, IFNA2, IFNA21, IFNA4, IFNA5, IFNA6, IFNA7, IFNA8, IFNB1, IFNE, IFNG, IFNK, IFNL1, IFNL2, IFNL3, IFNL4, IFNW1, IL10, IL11, IL12A, IL12B, IL13, IL15, IL16, IL17A, IL17B, IL17C, IL17D, IL17F, IL18, IL19, IL1A, IL1B, IL1F10, IL2, IL20, IL21, IL22, IL23A, IL24, IL25, IL26, IL27, IL3, IL31, IL32, IL33, IL34, IL36A, IL36B, IL36G, IL36RN, IL37, IL4, IL5, IL6, IL7, IL9, LEFTY1, LEFTY2, LIF, LTA, MIF, MSTN, NAMPT, NODAL, OSM, PF4, PF4V1, SCGB3A1, SECTM1, SLURP1, SPP1, THNSL2, THPO, TNF, TNFSF10, TNFSF11, TNFSF12, TNFSF13, TNFSF13B, TNFSF14, TNFSF15, TSLP, VSTM1, WNT1, WNT10A, WNT10B, WNT11, WNT16, WNT2, WNT2B, WNT3, WNT3A, WNT4, WNTSA, WNTSB, WNT6, WNT7A, WNT7B, WNT8A, WNT8B, WNT9A, WNT9B, XCL1, and XCL2; growth factors, wherein the growth factors are selected from the group consisting of AMH, ARTN, BTC, CDNF, CFC1, CFC1B, CHRDL1, CHRDL2, CLEC11A, CNMD, EFEMP1, EGF, EGFL6, EGFL7, EGFL8, EPGN, EREG, EYS, FGF1, FGF10, FGF16, FGF17, FGF18, FGF19, FGF2, FGF20, FGF21, FGF22, FGF23, FGF3, FGF4, FGF5, FGF6, FGF7, FGF8, FGF9, FRZB, GDNF, GFER, GKN1, HBEGF, HGF, IGF1, IGF2, INHA, INHBA, INHBB, INHBC, INHBE, INS, KITLG, MANF, MDK, MIA, NGF, NOV, NRG1, NRG2, NRG3, NRG4, NRTN, NTF3, NTF4, OGN, PDGFA, PDGFB, PDGFC, PDGFD, PGF, PROK1, PSPN, PTN, SDF1, SDF2, SFRP1, SFRP2, SFRP3, SFRP4, SFRP5, TDGF1, TFF1, TGFA, TGFB1, TGFB2, TGFB3, THBS4, TIMP1, VEGFA, VEGFB, VEGFC, VEGFD, and WISP3; growth factor binding proteins, wherein the growth factor binding proteins are selected from the group consisting of CHRD, CYR61, ESM1, FGFBP1, FGFBP2, FGFBP3, HTRA1, GHBP, IGFALS, IGFBP1, IGFBP2, IGFBP3, IGFBP4, IGFBP5, IGFBP6, IGFBP7, LTBP1, LTBP2, LTBP3, LTBP4, SOSTDC1, NOG, TWSG1, and WIF1; heparin binding proteins, wherein the heparin binding proteins are selected from the group consisting of ADA2, ADAMTSL5, ANGPTL3, APOB, APOE, APOH, COL5A1, COMP, CTGF, FBLN7, FN1, FSTL1, HRG, LAMC2, LIPC, LIPG, LIPH, LIPI, LPL, PCOLCE2, POSTN, RSPO1, RSPO2, RSPO3, RSPO4, SAA1, SLIT2, SOST, THBS1, and VTN; hormones, wherein the hormones are selected from the group consisting of ADCYAP1, ADIPOQ, ADM, ADM2, ANGPTL8, APELA, APLN, AVP, C1QTNF12, C1QTNF9, CALCA, CALCB, CCK, CGA, CGB1, CGB2, CGB3, CGBS, CGB8, COPA, CORT, CRH, CSH1, CSH2, CSHL1, ENHO, EPO, ERFE, FBN1, FNDCS, FSHB, GAL, GAST, GCG, GH, GH1, GH2, GHRH, GHRL, GIP, GNRH1, GNRH2, GPHA2, GPHB5, IAPP, INS, INSL3, INSL4, INSL5, INSL6, LHB, METRNL, MLN, NPPA, NPPB, NPPC, OSTN, OXT, PMCH, PPY, PRL, PRLH, PTH, PTHLH, PYY, RETN, RETNLB, RLN1, RLN2, RLN3, SCT, SPX, SST, STC1, STC2, TG, TOR2A, TRH, TSHB, TTR, UCN, UCN2, UCN3, UTS2, UTS2B, and VIP; neuroactive proteins, wherein the neuroactive proteins are selected from the group consisting of CARTPT, NMS, NMU, NPB, NPFF, NPS, NPVF, NPW, NPY, PCSK1N, PDYN, PENK, PNOC, POMC, PROK2, PTH2, PYY2, PYY3, QRFP, TAC1, and TAC3; and vasoactive proteins, wherein the vasoactive proteins are selected from the group consisting of AGGF1, AGT, ANGPT1, ANGPT2, ANGPTL4, ANGPTL6, EDN1, EDN2, EDN3, and NTS.

In an even more preferred embodiment, the protein is selected from the group consisting of cytokines; growth factors; hormones; and neuroactive proteins.

In a particular embodiment of the present invention the protein is selected from the group constisting of cytokines wherein the cytokines are selected from the group consisting of BMP1, BMP10, BMP15, BMP2, BMP3, BMP4, BMP5, BMP6, BMP7, BMP8A, BMP8B, C1QTNF4, CCL1, CCL11, CCL13, CCL14, CCL15, CCL16, CCL17, CCL18, CCL19, CCL2, CCL21, CCL22, CCL23, CCL24, CCL25, CCL26, CCL27, CCL28, CCL3, CCL3L1, CCL3L3, CCL4, CCL4L, CCL4L2, CCL5, CCL7, CCL8, CD40LG, CER1, CKLF, CLCF1, CNTF, CSF1, CSF2, CSF3, CTF1, CX3CL1, CXCL1, CXCL10, CXCL11, CXCL12, CXCL13, CXCL14, CXCL16, CXCL17, CXCL2, CXCL3, CXCL5, CXCL8, CXCL9, DKK1, DKK2, DKK3, DKK4, EDA, EBI3, FAM3B, FAM3C, FASLG, FLT3LG, GDF1, GDF10, GDF11, GDF15, GDF2, GDF3, GDF5, GDF6, GDF7, GDF9, GPI, GREM1, GREM2, GRN, IFNA1, IFNA13, IFNA10, IFNA14, IFNA16, IFNA17, IFNA2, IFNA21, IFNA4, IFNA5, IFNA6, IFNA7, IFNA8, IFNB1, IFNE, IFNG, IFNK, IFNL1, IFNL2, IFNL3, IFNL4, IFNW1, IL10, IL11, IL12A, IL12B, IL13, IL15, IL16, IL17A, IL17B, IL17C, IL17D, IL17F, IL18, IL19, IL1A, IL1B, IL1F10, IL2, IL20, IL21, IL22, IL23A, IL24, IL25, IL26, IL27, IL3, IL31, IL32, IL33, IL34, IL36A, IL36B, IL36G, IL36RN, IL37, IL4, IL5, IL6, IL7, IL9, LEFTY1, LEFTY2, LIF, LTA, MIF, MSTN, NAMPT, NODAL, OSM, PF4, PF4V1, SCGB3A1, SECTM1, SLURP1, SPP1, THNSL2, THPO, TNF, TNFSF10, TNFSF11, TNFSF12, TNFSF13, TNFSF13B, TNFSF14, TNFSF15, TSLP, VSTM1, WNT1, WNT10A, WNT10B, WNT11, WNT16, WNT2, WNT2B, WNT3, WNT3A, WNT4, WNTSA, WNTSB, WNT6, WNT7A, WNT7B, WNT8A, WNT8B, WNT9A, WNT9B, XCL1, and XCL2; growth factors, wherein the growth factors are selected from the group consisting of AMH, ARTN, BTC, CDNF, CFC1, CFC1B, CHRDL1, CHRDL2, CLEC11A, CNMD, EFEMP1, EGF, EGFL6, EGFL7, EGFL8, EPGN, EREG, EYS, FGF1, FGF10, FGF16, FGF17, FGF18, FGF19, FGF2, FGF20, FGF21, FGF22, FGF23, FGF3, FGF4, FGF5, FGF6, FGF7, FGF8, FGF9, FRZB, GDNF, GFER, GKN1, HBEGF, HGF, IGF1, IGF2, INHA, INHBA, INHBB, INHBC, INHBE, INS, KITLG, MANF, MDK, MIA, NGF, NOV, NRG1, NRG2, NRG3, NRG4, NRTN, NTF3, NTF4, OGN, PDGFA, PDGFB, PDGFC, PDGFD, PGF, PROK1, PSPN, PTN, SDF1, SDF2, SFRP1, SFRP2, SFRP3, SFRP4, SFRP5, TDGF1, TFF1, TGFA, TGFB1, TGFB2, TGFB3, THBS4, TIMP1, VEGFA, VEGFB, VEGFC, VEGFD, and WISP3; hormones, wherein the hormones are selected from the group consisting of ADCYAP1, ADIPOQ, ADM, ADM2, ANGPTL8, APELA, APLN, AVP, C1QTNF12, C1QTNF9, CALCA, CALCB, CCK, CGA, CGB1, CGB2, CGB3, CGBS, CGB8, COPA, CORT, CRH, CSH1, CSH2, CSHL1, ENHO, EPO, ERFE, FBN1, FNDCS, FSHB, GAL, GAST, GCG, GH, GH1, GH2, GHRH, GHRL, GIP, GNRH1, GNRH2, GPHA2, GPHB5, IAPP, INS, INSL3, INSL4, INSL5, INSL6, LHB, METRNL, MLN, NPPA, NPPB, NPPC, OSTN, OXT, PMCH, PPY, PRL, PRLH, PTH, PTHLH, PYY, RETN, RETNLB, RLN1, RLN2, RLN3, SCT, SPX, SST, STC1, STC2, TG, TOR2A, TRH, TSHB, TTR, UCN, UCN2, UCN3, UTS2, UTS2B, and VIP; and neuroactive proteins, wherein the neuroactive proteins are selected from the group consisting of CARTPT, NMS, NMU, NPB, NPFF, NPS, NPVF, NPW, NPY, PCSK1N, PDYN, PENK, PNOC, POMC, PROK2, PTH2, PYY2, PYY3, QRFP, TAC1, and TAC3.

In another particular embodiment of the present invention the protein is selected from the group constisting of cytokines wherein the cytokines are selected from the group consisting of BMP-2, BMP-4, CNTF, MSTN, IFNG, IL6, SPP1; growth factors, wherein the growth factors are selected from the group consisting of EGF, FGF1, GDNF, IGF1, IGF2, NTF3, TGFB1; hormones, wherein the hormones are selected from the group consisting of EPO, FBN1, GH, GHRH, OSTN, UCN; and neuroactive proteins, wherein the neuroactive proteins are selected from the group consisting of NPFF, NPY, PNOC, POMC.

In another particular embodiment of the present invention the protein is selected from the group constisting of cytokines wherein the cytokines are selected from the group consisting of BMP-2, BMP-4, CNTF, MSTN, IFNG, IL4, IL6, IL10, SPP1; growth factors, wherein the growth factors are selected from the group consisting of EGF, FGF1, GDNF, IGF1, IGF2, NTF3, TGFB1; hormones, wherein the hormones are selected from the group consisting of EPO, FBN1, GH, GHRH, OSTN, UCN, INS; and neuroactive proteins, wherein the neuroactive proteins are selected from the group consisting of NPFF, NPY, PNOC, POMC.

In a more particular embodiment of the present invention the protein is selected from the group constisting of growth factors. In an even more particular embodiment of the present invention the protein is selected from the group constisting of growth factors, wherein the growth factors are selected from the group consisting of AMH, ARTN, BDNF, BTC, CDNF, CFC1, CFC1B, CHRDL1, CHRDL2, CLEC11A, CNMD, EFEMP1, EGF, EGFL6, EGFL7, EGFL8, EPGN, EREG, EYS, FGF1, FGF10, FGF16, FGF17, FGF18, FGF19, FGF2, FGF20, FGF21, FGF22, FGF23, FGF3, FGF4, FGF5, FGF6, FGF7, FGF8, FGF9, FRZB, GDNF, GFER, GKN1, HBEGF, HGF, IGF1, IGF2, INHA, INHBA, INHBB, INHBC, INHBE, INS, KITLG, MANF, MDK, MIA, NGF, NOV, NRG1, NRG2, NRG3, NRG4, NRTN, NTF3, NTF4, OGN, PDGFA, PDGFB, PDGFC, PDGFD, PGF, PROK1, PSPN, PTN, SDF1, SDF2, SFRP1, SFRP2, SFRP3, SFRP4, SFRP5, TDGF1, TFF1, TGFA, TGFB1, TGFB2, TGFB3, THBS4, TIMP1, VEGFA, VEGFB, VEGFC, VEGFD, and WISP3.

In another even more particular embodiment of the present invention the protein is selected from the group constisting of growth factors, wherein the growth factors are selected from the group consisting of EGF, FGF1, GDNF, IGF1, IGF2, NTF3, TGFB1. Most particular, the protein is IGF1, preferably human IGF1.

In an even much more particular embodiment of the present invention the protein is selected from the group consisting of cytokines; growth factors; and hormones, wherein preferably the the cytokines are selected from the group consisting of BMP-2, BMP-4, CNTF, MSTN, IFNG, IL4, IL6, IL10, SPP1; the growth factors are selected from the group consisting of EGF, FGF1, GDNF, IGF1, IGF2, NTF3, TGFB1; and the hormones are selected from the group consisting of EPO, FBN1, GH, GHRH, OSTN, UCN, INS. Most particular the protein is selected from the group consisting of insulin growth factor 1 (IGF1), insulin (INS), erythropoietin (EPO), Interleukin-4 (IL-4) and interleukin-10 (IL-10).

In one embodiment of the present invention the mRNA is naked mRNA. In a preferred embodiment, the mRNA comprises an antireverse CAP analog such as m7G(5′)G, m7GpppG cap, an internal ribosome entry site (IRES) and/or a polyA tail at the 3′ end in particular in order to improve translation. The mRNA can have further regions promoting translation known to the skilled person.

In a preferred embodiment of the present invention the mRNA contains a combination of modified and unmodified nucleotides. In a more preferred embodiment, in such a modified mRNA 1 to 100%, preferably 10 to 100%, more preferably 50 to 100%, even more preferably 90 to 100%, most preferably 100% of the uridine nucleotides are modified. The adenosine-, guanosine-, and cytidine-containing nucleotides can be unmodified or partially modified, and they are preferably present in unmodified form. Preferably the content of the modified uridine nucleotides in the mRNA lies in a range from 5 to 25%. In a particularly preferred embodiment of the present inventionthe modified uridine nucleotides are N¹-Methylpseudouridines. In a more particularly preferred embodiment of the present invention the mRNA contains a combination of modified and unmodified nucleotides, wherein in such a modified mRNA 1 to 100%, preferably 10 to 100%, more preferably 50 to 100%, even more preferably 90 to 100%, most preferably 100% of the uridine nucleotides are N¹-Methylpseudouridines.

In a more preferred embodiment of the present invention the mRNA, is an mRNA which is codon optimized and contains a combination of modified and unmodified nucleotides. In a more preferred embodiment, in such a modified mRNA 1 to 100%, preferably 10 to 100%, more preferably 50 to 100%, even more preferably 90 to 100%, most preferably 100% of the uridine nucleotides are modified. The adenosine-, guanosine-, and cytidine-containing nucleotides can be unmodified or partially modified, and they are preferably present in unmodified form. Preferably the content of the modified uridine nucleotides in the mRNA lies in a range from 5 to 25%. In a particularly preferred embodiment of the present invention the modified uridine nucleotides are N¹-Methylpseudouridines. In a more particularly preferred embodiment of the present invention the RNA is mRNA which contains a combination of modified and unmodified nucleotides, wherein in such a modified mRNA 1 to 100%, preferably 10 to 100%, more preferably 50 to 100%, even more preferably 90 to 100%, most preferably 100% of the uridine nucleotides are N′-Methylpseudouridines.

In a preferred embodiment of the present invention the mRNA comprises a nucleic acid sequence encoding human insulin-like growth factor 1 (IGF1) as protein, more preferably the mRNA is naked mRNA comprising a nucleic acid sequence encoding human insulin-like growth factor 1 (IGF1) as protein. In this preferred embodiment of the present invention the mRNA comprises a nucleic acid sequence encoding the mature human IGF-1.

In a more preferred embodiment of the present invention the mRNA comprises a nucleic acid sequence encoding the propeptide of IGF1, preferably the propeptide of human IGF1, and a nucleic acid sequence encoding the mature protein of IGF1, preferably the mature protein of human IGF1, and does not comprise a nucleic acid sequence encoding an E-peptide of IGF1, preferably does not comprise a nucleic acid sequence encoding a human E-peptide of IGF1. In a further more preferred embodiment of the present invention the mRNA comprises a nucleic acid sequence encoding the propeptide of IGF1, preferably the propeptide of human IGF1, a nucleic acid sequence encoding the mature protein of IGF1, preferably the mature protein of human IGF1. Preferably the mRNA does not comprise a nucleic acid sequence encoding an E-peptide of IGF1, more preferably does not comprise a nucleic acid sequence encoding a human E-peptide of IGF1. In a further more preferred embodiment of the present invention the mRNA comprises a nucleic acid sequence encoding the propeptide of IGF1, preferably the propeptide of human IGF1, a nucleic acid sequence encoding the mature protein of IGF1, preferably the mature protein of human IGF1 and a nucleic acid sequence encoding the signal peptide of the brain-derived neurotrophic factor (BDNF). Preferably the mRNA does not comprise a nucleic acid sequence encoding an E-peptide of IGF1, more preferably does not comprise a nucleic acid sequence encoding a human E-peptide of IGF1.

In an even more preferred embodiment of the present invention the mRNA comprises a nucleotide acid sequence encoding the propeptide (also called pro-domain) of IGF1, preferably of human IGF1 having 27 amino acids, and a nucleotide sequence encoding the mature IGF1, preferably the mature human IGF1 having 70 amino acids, and preferably does not comprise a nucleotide sequence encoding an E-peptide of IGF1, preferably does not comprise a nucleic acid sequence encoding a human E-peptide of IGF1.

In a further even more preferred embodiment of the present invention the mRNA comprises a nucleotide acid sequence encoding the propeptide (also called pro-domain) of IGF1, preferably of human IGF1 having 27 amino acids, a nucleotide sequence encoding the mature IGF1, preferably the mature human IGF1 having 70 amino acids and a nucleic acid sequence encoding the signal peptide of the brain-derived neurotrophic factor (BDNF). Preferably the mRNA does not comprise a nucleotide sequence encoding an E-peptide of IGF1, more preferably does not comprise a nucleic acid sequence encoding a human E-peptide of IGF1.

In a particular preferred embodiment of the present invention the mRNA comprises a nucleic acid sequence encoding the propeptide (also called pro-domain) of human IGF1 having 27 amino acids, and a nucleotide acid sequence encoding the mature human IGF1 having 70 amino acids and preferably does not comprise a nucleotide sequence encoding an E-peptide (also called E-domain) of human IGF1, wherein the nucleotide sequence encoding the propeptide (also called pro-domain) of human IGF1 having 27 amino acids, and the nucleotide sequence encoding the mature human IGF1 having 70 amino acids and the nucleotide sequence encoding the E-peptides are as referred to in the Uniprot database as UniProtKB-P05019 and in the Genbank database as NM_000618.4, NM_001111285.2 and NM_001111283.2, respectively.

In an even more particular preferred embodiment of the present invention the mRNA comprises a nucleic acid sequence encoding the propeptide (also called pro-domain) of human IGF1 having 27 amino acids as shown in SEQ ID NO: 38 and a nucleotide acid sequence encoding the mature human IGF1 having 70 amino acids as shown in SEQ ID NO: 39, and preferably does not comprise a nucleotide sequence encoding an E-peptide (also called E-domain) of human IGF1.

In a further even more particular preferred embodiment of the present invention the mRNA comprises a nucleic acid sequence encoding the propeptide (also called pro-domain) of human IGF1 having 27 amino acids as shown in SEQ ID NO: 38, a nucleotide acid sequence encoding the mature human IGF1 having 70 amino acids as shown in SEQ ID NO: 39 and a nucleic acid sequence encoding the signal peptide of the brain-derived neurotrophic factor (BDNF), preferably, a nucleotide acid sequence encoding the signal peptide of the brain-derived neurotrophic factor (BDNF) as shown in SEQ ID NO: 30. Preferably the mRNA does not comprise a nucleotide sequence encoding an E-peptide (also called E-domain) of human IGF1.

In a particular preferred embodiment of the present invention the mRNA comprising a nucleic acid sequence encoding human insulin-like growth factor 1 (IGF1) and the signal peptide of the brain-derived neurotrophic factor (BDNF) comprises a nucleic acid sequence as shown in SEQ ID NO: 8.

In a further particular preferred embodiment of the present invention the mRNA comprising a nucleic acid sequence encoding human insulin-like growth factor 1 (IGF1) and the signal peptide of the brain-derived neurotrophic factor (BDNF) comprises a nucleic acid sequence transcribed from the DNA sequence as shown in SEQ ID NO: 7. Preferably the nucleic acid sequence is transcribed from the DNA sequence as shown in SEQ ID NO: 7 in vitro.

In a more particular preferred embodiment of the present invention the mRNA comprising a nucleic acid sequence encoding human insulin-like growth factor 1 (IGF1) and the signal peptide of the brain-derived neurotrophic factor (BDNF) comprises a nucleic acid sequence as shown in SEQ ID NO: 8 wherein preferably 1 to 100%, more preferably 50 to 100%, even more preferably 90 to 100%, most preferably 100% of the uridine nucleotides are N¹-Methylpseudouridines.

In a further more particular preferred embodiment of the present invention the mRNA comprising a nucleic acid sequence encoding human insulin-like growth factor 1 (IGF1) and the signal peptide of the brain-derived neurotrophic factor (BDNF) comprises a nucleic acid sequence transcribed from the DNA sequence as shown in SEQ ID NO: 7, wherein preferably 1 to 100%, more preferably 50 to 100%, even more preferably 90 to 100%, most preferably 100% of the uridine nucleotides are N¹-Methylpseudouridines. In this embodiment the nucleotide sequence is preferably transcribed from the DNA sequence as shown in SEQ ID NO: 7 in vitro, whereas as uridine nucleotides only N¹-Methylpseudouridine-5′-Triphosphate (N¹-Methylpseudo-UTP) i.e. 100% N¹-Methylpseudo-UTP is used for the transcription from the DNA sequence as shown in SEQ ID NO: 7.

In a preferred embodiment of the present invention the signal peptide of the brain-derived neurotrophic factor (BDNF) is the signal peptide of the human BDNF, more preferably the signal peptide as shown in SEQ ID NO: 31, in particular the signal peptide of the human BDNF encoded by the nucleic acid sequence as shown in SEQ ID NO: 30.

In a more preferred embodiment of the present invention the mRNA comprises a nucleic acid sequence encoding in the following order from 5′ to 3′:

i) the signal peptide of the brain-derived neurotrophic factor (BDNF);
ii) optionally a pro-domain of the protein; and
iii) the mature protein.

In an even more preferred embodiment of the present invention the mRNA comprises a nucleic acid sequence encoding in the following order from 5′ to 3′:

i) the signal peptide of the brain-derived neurotrophic factor (BDNF);
ii) optionally a pro-domain of human IGF; and
iii) the mature human IGF.

In a preferred embodiment of the present invention the signal peptide of the brain-derived neurotrophic factor (BDNF) replaces the natural signal peptide of the protein.

In a further aspect the present invention provides a transcription unit, an expression vector or a gene therapy vector comprising a nucleic acid encoding a protein and a signal peptide, wherein the amino acids 1-9 of the N-terminal end of the amino acid sequence of the signal peptide have an average hydrophobic score of above 2, wherein the signal peptide is selected from the group consisting of

i) a signal peptide heterologous to said protein, wherein the signal peptide heterologous to said protein is optionally modified by insertion, deletion and/or substitution of at least one amino acid, with the proviso that said protein is not an oxidoreductase;
ii) a signal peptide homologous to said protein, wherein the signal peptide homologous to said protein is modified by insertion, deletion and/or substitution of at least one amino acid; and
iii) a naturally occurring amino acid sequence which does not have the function of a signal peptide in nature, wherein the naturally occurring amino acid sequence is optionally modified by insertion, deletion and/or substitution of at least one amino acid.

In a further aspect the present invention provides a transcription unit, an expression vector or a gene therapy vector comprising a nucleic acid sequence encoding a protein and a signal peptide heterologous to said protein, wherein the signal peptide heterologous to said protein is the signal peptide of the brain-derived neurotrophic factor (BDNF) and wherein the protein is not an oxidoreductase, preferably not a thioredoxin, more preferably not rod-derived cone viability factor. As regards the signal peptide of the brain-derived neurotrophic factor (BDNF) and the protein, the same applies as has been set forth herein elsewhere.

In a further aspect the present invention provides a transcription unit, an expression vector or a gene therapy vector comprising a nucleic acid sequence encoding a protein and a signal peptide heterologous to said protein, wherein the signal peptide heterologous to said protein is the signal peptide of the brain-derived neurotrophic factor (BDNF) and wherein the protein is selected from the group consisting of carboxypeptidases; cytokines; extracellular ligands and transporters; extracellular matrix proteins; glucosidases; glycosyltransferases; growth factors; growth factor binding proteins; heparin binding proteins; hormones; hydrolases; immunoglobulins; isomerases; kinases; lyases; metalloenzyme inhibitors; metalloproteases; milk proteins; neuroactive proteins; proteases; protease inhibitors; protein phosphatases; esterases; transferases; and vasoactive proteins. As regards the signal peptide of the brain-derived neurotrophic factor (BDNF) and the protein, the same applies as has been set forth herein elsewhere.

In a further aspect the present invention provides a therapeutic composition comprising the mRNA and/or the transcription unit, the expression vector or the gene therapy vector as described above. As regards the signal peptide of the brain-derived neurotrophic factor (BDNF) and the protein, the same applies as has been set forth herein elsewhere. Usually the mRNA of the present invention is provided as therapeutic composition, which is preferably a liquid composition. A liquid composition is any composition in which the mRNA is present in solution in a liquid. In one embodiment of the present inventionthe mRNA is solved in water, or a buffered or unbuffered aqueous solution. The solution is preferably an aqueous solution. Thus, the liquid may be water, preferably sterile water, more preferably “water for injection” (WFI) or any other buffered or unbuffered aqueous solution. In one embodiment of the present inventionthe liquid composition is an unbuffered solution, preferably a salt solution, more preferably a salt solution of a pharmaceutically acceptable salt, even more preferably a NaCl solution, i.e. saline. Preferably, the salt solution is isotonic and even more preferably it shows a physiological pH value. In a preferred embodiment of the present invention the solution in which the mRNA is contained is a buffered solution. Preferably, such a solution is isotonic to blood. In principle any buffer which effectively buffers in the physiological range, in particular in the range of pH 3.0 to 10.5 and more preferably pH 4.0 to 9.0, can be used. Preferable buffers are acetate, phosphate, phosphate buffered saline (PBS), carbonate, lactate and citrate buffers or Ringer's solution, preferably phosphate buffered saline (PBS). Thus in a more preferred embodiment of the present invention the solution in which the mRNA is contained is phosphate buffered saline (PBS).

The concentration of the mRNA in the therapeutic composition is not particularly crucial and can be adjusted as required. Preferably, the concentration lies in the range of 0.05 to 20.0 μg/μl, more preferably in the range of 0.1 to 10.0 μg/μl, even more preferably in the range of 0.2 to 5 μg/1, in particular in the range of 0.4 to 2.0 μg/μl, more particular in the range of 0.6 to 1.5 μg/μl, even more particular in the range of 0.80 to 1.20 μg/μl. Particular preferred is a range of 0.01 μg to 0.1 g, preferably of 0.1 μg to 0.01 g, more preferably of 0.5 μg to 1 mg, even more preferably of 0.5 μg to 10 μg.

In a further aspect the present invention provides a kit comprising the mRNA and/or the transcription unit, the expression vector or the gene therapy vector or the therapeutic composition as described above, and instructions, optionally a vector map, optionally a host cell, optionally a cultivation medium for the cultivation of a host cell, and/or optionally a selection medium for selecting and cultivating a transfected host cell. The kit of the invention may be provided in (or in form of) a kit of contents. The kit may further comprise one or more of the components of the therapeutic composition of the invention, for example in one or more separate containers. For example, the kit may comprise the mRNA (e.g. in dried form), a solubilizer and (buffered or unbuffered) aqueous solution, for example in one, two or three (or more) separate containers, respectively. The kit may also comprise the instruction manual or instruction leaflet.

In a further aspect the present invention provides the mRNA, the transcription unit, the expression vector or the gene therapy vector, the therapeutic composition or the kit as described above for use as a medicament. As regards the signal peptide, e.g. the signal peptide of the brain-derived neurotrophic factor (BDNF) and the protein, the same applies as has been set forth herein elsewhere.

In a further aspect the present invention provides a mRNA or a therapeutic composition comprising or containing mRNA for use in a method of treating skeletal muscle injury. The present invention provides also the use of a mRNA or a therapeutic composition comprising or containing mRNA for the manufacture of a medicament for treating skeletal muscle injury in a subject.

The present invention provides also a method of treating a skeletal muscle injury in a subject, which method comprises administering to the subject a mRNA or a therapeutic composition comprising or containing mRNA.

Skeletal muscle injuries such as muscle ruptures are one of the most common injuries occurring in sports, their frequency varying from 10-55% of all sustained injuries. Muscle injuries can be caused by eccentric muscle contractions, elongations and muscle overload. Over 90% of all sports-related injuries are caused by either eccentric muscle contractions, elongations or muscle overload. Skeletal muscle injury occurs when a muscle is subjected to a sudden, heavy compressive force, such as a direct blow. In muscle ruptures, the muscle is subjected to an excessive and eccentric tensile force leading to the overstraining of the myofibres and, consequently, to their rupture near the myotendinous junction (MTJ). Muscle ruptures are one of the most common complaints treated by physicians and account for the majority of all sports-related injuries. Injuries of the hamstring muscle complex (HMC) often affect athletes participating in sports that force rapid acceleration and deceleration while running and require eccentric muscle contraction. Mild injuries can easily be handled by conservative treatment, and the more devastating injury is the total rupture of the hamstring muscles. Hamstring muscle ruptures are treated conservatively or surgically depending on how they are classified. There are mild, moderate, or severe ruptures. While mild-to-moderate ruptures can be treated conservatively, severe ruptures are a clear indication for surgical treatment. Conservative treatment is dictated by the clinical presentation and starts immediately with cryotherapy, compressive bandaging, immobilization, and non-steroidal, anti-inflammatory drugs before elastic banding, and physiotherapy once the patient is comfortable. Therapeutic ultrasound is widely discussed as a therapeutic option, but no significant effects on the final outcome of regeneration were found. Within 2 weeks, there should be a clear decrease in pain so that physiotherapy can be increased to include active exercise as mentioned above. However, it is recognised in the field that surgical intervention is not without risk, and candidates must be carefully selected (Järvinen T A, Järvinen T L, Kääriäinen M, Aärimaa V, Vaittinen S, Kalimo H, Järvinen M (2007) Muscle injuries: optimising recovery. Best Pract Res Clin Rheumatol 21(2):317-331. DOI:10.1016/j.berh.2006.12.004; Horst K, Dienstknecht T, Sellei R M, Pape H C (2014) Partial rupture of the hamstring muscle complex: a literature review on treatment options. Eur J Orthop Surg Traumatol 24(3):285-9. DOI:10.1007/s00590-013-1315-x). The current therapeutic options offer little beyond the body's own healing processes, and in fact it is possible that non-steroidal anti-inflammatory drugs (NSAIDs) impair the healing process.

With no effective pharmaceutical therapies available today, the unmet medical need is high. In particular there is a need for providing effective methods for treating skeletal muscle injury which accelerates the recovery process and result in an increase of the function of the injured muscle.

In a preferred embodiment of the present invention the mRNA for use in a method of treating skeletal muscle injury is mRNA encoding a growth factor, preferably mRNA encoding human insulin-like growth factor 1 (IGF1). The mRNA encoding the growth factor comprises usually a nucleic sequence encoding a signalling peptide, optionally a nucleic sequence encoding the propeptide of the growth factor and a nucleic sequence encoding the mature growth factor. The mRNA encoding human IGF1 comprises preferably a nucleic sequence encoding a signalling peptide, optionally a nucleic sequence encoding the propeptide of human IGF1 and a nucleic sequence encoding the mature human IGF1, even more preferably a nucleic sequence encoding a signalling peptide, a nucleic sequence encoding the propeptide of human IGF1 and a nucleic sequence encoding the mature human IGF1 and, does not comprise a nucleic sequence encoding an E-peptide of human IGF1. The signalling peptide comprised by the mRNA encoding a growth factor can be a signalling peptide homologous to the growth factor i.e. the signalling peptide of the growth factor or can be a signalling peptide heterologous to the growth factor and is preferably a signalling peptide heterologous to the growth factor, more preferably the signal peptide of the brain-derived neurotrophic factor (BDNF), in particular the signal peptide of human BDNF. The signalling peptide comprised by the mRNA encoding human IGF1 can be a signalling peptide homologous to human IGF1 i.e. the signalling peptide of human IGF1 or can be a signalling peptide heterologous to human IGF1 and is preferably a signalling peptide heterologous to human IGF1, more preferably the signal peptide of the brain-derived neurotrophic factor (BDNF), in particular the signal peptide of human BDNF.

Thus in a more preferred embodiment of the present invention the mRNA for use in a method of treating skeletal muscle injury is mRNA encoding human insulin-like growth factor 1 (IGF1) which comprises a nucleic sequence encoding the signal peptide of the brain-derived neurotrophic factor (BDNF) in particular the signal peptide of human BDNF, optionally a nucleic sequence encoding the propeptide of human IGF1 and a nucleic sequence encoding the mature human IGF-1. In an even more preferred embodiment of the present invention the mRNA for use in a method of treating skeletal muscle injury is mRNA encoding human insulin-like growth factor 1 (IGF1) which comprises a nucleic sequence encoding the signal peptide of the brain-derived neurotrophic factor (BDNF) in particular the signal peptide of human BDNF, optionally a nucleic sequence encoding the propeptide of human IGF1 and a sequence encoding the mature human IGF-1 and does not comprise a nucleic sequence encoding an E-peptide of human IGF1.

Thus in a further aspect the present invention provides a mRNA comprising a nucleic acid sequence encoding

i) IGF1, preferably human IGF1; and
ii) the signal peptide of the brain-derived neurotrophic factor (BDNF), preferably the signal peptide of human BDNF, for use in a method for treating skeletal muscle injury.

The present invention provides also the use of a mRNA comprising a nucleic acid sequence encoding

i) IGF1, preferably human IGF1; and
ii) the signal peptide of the brain-derived neurotrophic factor (BDNF), preferably the signal peptide of human BDNF, for the manufacture of a medicament for treating skeletal muscle injury in a subject.

The present invention provides also a method of treating a skeletal muscle injury in a subject, which method comprises administering to the subject a mRNA comprising a nucleic acid sequence encoding

i) IGF1, preferably human IGF1; and
ii) the signal peptide of the brain-derived neurotrophic factor (BDNF), preferably the signal peptide of human BDNF.

Preferably the present invention provides a mRNA comprising a nucleic acid sequence encoding

i) the mature IGF1, preferably the mature human IGF1;
ii) optionally the pro-domain of IGF1, preferably of human IGF1;
iii) the signal peptide of the brain-derived neurotrophic factor (BDNF), preferably the signal peptide of human BDNF, for use in a method for treating skeletal muscle injury.

The present invention provides also the use of a mRNA comprising a nucleic acid sequence encoding

i) the mature IGF1, preferably the mature human IGF1;
ii) optionally the pro-domain of IGF1, preferably of human IGF1;
iii) the signal peptide of the brain-derived neurotrophic factor (BDNF), preferably the signal peptide of human BDNF, for the manufacture of a medicament for treating skeletal muscle injury in a subject.

The present invention provides also a method of treating a skeletal muscle injury in a subject, which method comprises administering to the subject a mRNA comprising a nucleic acid sequence encoding

i) the mature IGF1, preferably the mature human IGF1;
ii) optionally the pro-domain of IGF1, preferably of human IGF1;
iii) the signal peptide of the brain-derived neurotrophic factor (BDNF), preferably the signal peptide of human BDNF.

As regards the mRNA comprising a nucleic acid sequence encoding human insulin-like growth factor 1 (IGF1) and the signal peptide of the brain-derived neurotrophic factor (BDNF) for use in a method for treating skeletal muscle injury the same applies as has been set forth herein elsewhere. In a particular preferred embodiment of the present invention the mRNA comprising a nucleic acid sequence encoding human insulin-like growth factor 1 (IGF1) and the signal peptide of the brain-derived neurotrophic factor (BDNF) comprises a nucleic acid sequence as shown in SEQ ID NO: 8. In a further particular preferred embodiment of the present invention the mRNA comprising a nucleic acid sequence encoding human insulin-like growth factor 1 (IGF1) and the signal peptide of the brain-derived neurotrophic factor (BDNF) comprises a nucleic acid sequence transcribed from the DNA sequence as shown in SEQ ID NO: 7. Preferably the nucleic acid sequence is transcribed from the DNA sequence as shown in SEQ ID NO: 7 in vitro.

The mRNA and/or the therapeutic composition can be applied to cells and tissues e.g. skeletal muscles by means known to the person skilled in the art, preferably by injection, more preferably by intra-muscular injection, typically by using a syringe with a needle. In principle any commercially available syringe in combination with a needle can be used for this purpose. Preferred are hypodermic needles. The diameter of a needle is indicated by the needle gauge (G; according to the Stub's Needle Gauge). Typically needles in medical use range from 7 G (the largest) to 33 G (the smallest) can be used.

In some embodiments, the mRNA and/or the therapeutic composition can be delivered to a cell via direct DNA transfer (Wolff et al. (1990) Science 247, 1465-1468). The mRNA and/or the therapeutic composition can be delivered to cells following mild mechanical disruption of the cell membrane, temporarily permeabilizing the cells. Such a mild mechanical disruption of the membrane can be accomplished by gently forcing cells through a small aperture (Sharei et al. PLOS ONE (2015) 10(4), e0118803). In another embodiment, the mRNA and/or the therapeutic composition can be delivered to a cell via liposome-mediated DNA transfer (e.g., Gao & Huang (1991) Biochem. Biophys. Res. Comm. 179, 280-285, Crystal (1995) Nature Med. 1, 15-17, Caplen et al. (1995) Nature Med. 3, 39-46). The term “liposome” can encompass a variety of single and multilamellar lipid vehicles formed by the generation of enclosed lipid bilayers or aggregates. The mRNA can be encapsulated in the aqueous interior of a liposome, interspersed within the lipid bilayer of a liposome, attached to a liposome via a linking molecule that is associated with both the liposome and the oligonucleotide, entrapped in a liposome, or complexed with a liposome.

In one embodiment of the present invention the RNA or the therapeutic composition is administered directly into the skeletal muscle (preferably by injection) in the form of a therapeutic i.e. a liquid composition wherein the RNA is contained as naked RNA. As regards the way of administration and the characteristics of the composition and the RNA contained therein, the same applies as has been set forth herein elsewhere. In a preferred embodiment, the liquid composition and mRNA, respectively, of the present invention is to be administered directly into the skeletal muscle. In this context, the most preferred way of administration is injection, i.e. intra-muscular injection.

It is, in principle, envisaged in the context of the invention to administer the mRNA and the therapeutic composition, respectively, as early as possible, i.e. at the earliest possible stage of the skeletal muscle injury. For example, this stage is once (a) first symptom(s) have/has been observed (e.g. pain). However, any possible point of time after the diagnosis is possible and worthwhile and, hence, envisaged in accordance with the invention. For example, in case there is a surgical intervention (for example following a muscle rupture) the mRNA and the therapeutic composition, respectively, may be administered already during, but at least shortly after, the surgical intervention.

In one embodiment, the mRNA and the therapeutic composition, respectively, is to be administered during or even before the inflammatory and early proliferative phase, respectively, of skeletal muscle regeneration. For example, administration may be during day 0 to day 10, preferably during day 0 to day 7, post injury. More specifically, administration may be at day 0, 1, 2, 3, 4, 5, 6 or 7 post injury. Preferably, administration is at day 1 and even more preferably at day 0 post injury. In a preferred embodiment, the therapeutic composition is to be administered before the inflammatory phase which follows the said skeletal muscle injury. Particular preferred is administration at day 1 post injury which is repeated at day 4 post injury.

The administration of the mRNA and the therapeutic composition, respectively, in accordance with the invention may, for example depending on the course of the injury to be treated, be repeated at least once but preferably several times (for example 3 to 5 times). The repeated administration may be after 1, 2, 3, 4, 5, 6, 7, 8, or 9 days, preferably after day 2, 3, 4, 5, 6, 7, more preferably after day 3, 4 or 5. The repeated administration may be every few weeks (for example every 1, 2, 3, or 4 weeks) up to every few days (for example every 1, 2, 3, 4, 5 or 6 days), preferably every 2 or 3 days.

The mRNA or the therapeutic composition of the invention can be administered to a patient at a suitable dose. The dosage regimen can be determined by the attending physician, for example based on clinical factors. As is well known in the medical arts, dosages for any one patient depend upon many factors, including the patient's size, body surface area, age, the particular compound to be administered, sex, time and route of administration, general health, and other drugs being administered concurrently. However, the skilled person/the attending physician is readily in a position to (a) deduce (therapeutically) effective concentration(s) and/or dosages of the active substance(s) to be administered, e. g. in vivo or ex vivo. Corresponding samples may be taken from, for example, skeletal muscle (e.g. by a suitable probe) and the active compounds (naked RNA) may be detected and their corresponding concentrations may be determined in said samples, for example by HPLC.

A typical dose of active substances (e.g. mRNA) can be, for example, in the range of 1 ng to several grams, preferably in the range of 0.1 μg to 1 g, preferably in the range of 1 μg to 0.1 g, more preferably in the range of 10 μg to 1 mg, even more preferably in the range of 15 μg to 0.5 mg and most preferably in the range of 20 μg to 100 μg. Particular preferred is a range of 0.01 μg to 0.1 g, preferably of 0.1 μg to 0.01 g, more preferably of 0.5 μs to 1 mg, even more preferably of 0.5 μg to 10 μg. This particularly applies to a human patient. Applied to (m)RNA therapy, the dosage of an (m)RNA for expression should correspond to this range; however, doses below or above this exemplary range are, in principle, also envisioned, especially considering the aforementioned factors. Generally, the regimen as a regular administration of the therapeutic composition should be in the range of 0.1 μg to 10 mg units, preferably in the range of 1 μg to 1 mg units, more preferably in the range of 10 μg to 0.1 mg units per kilogram of body weight per day. Again, this is particularly applied to a human patient. Progress can be monitored by periodic assessment. Dosages may vary but a preferred dosage for administration by injection of (m)RNAs as constituents of the liquid composition of the present invention is from approximately 10⁵ to 10¹⁵ copies of the (m)RNA molecule per injection. Again, this particularly applies to a human patient.

In particular, the therapeutic composition of the invention is envisaged to be administered to a patient, preferably to a human patient/a human. However, the herein described skeletal muscle injuries may also be treated (or prevented) in a non-human animal subject/patient like, for example, a pet (e.g. dog, cat, rabbit, rat and mouse), a cattle (e.g. cow, pig, sheep), a horse (e.g. a race horse) or pony, a camel (e.g. a race camel) or a bird (e.g. chicken, turkey, parrot).

In particular, the therapeutic composition comprising mRNA is therapeutically active in the healing process of an injury, a disorder and/or a disease, such as e.g. skeletal muscle injury. In a more particular preferred embodiment of the present invention the mRNA encoding insulin-like growth factor 1 (IGF1) for use as a medicament comprises a nucleic acid sequence transcribed from the DNA sequence of SEQ ID NO: 7. In an even more particular preferred embodiment of the present invention the mRNA encoding insulin-like growth factor 1 (IGF1) for use as a medicament comprises the nucleic acid sequence of SEQ ID NO: 8.

Any of the therapeutic compositions of the invention may be provided together with an instruction manual or instruction leaflet. The instruction manual/leaflet may comprise guidance for the skilled person/attending physician how to treat (or prevent) a disease or disorder as described herein (skeletal muscle injury) in accordance with the invention. In particular, the instruction manual/leaflet may comprise guidance as to the herein described mode of delivery/administration and delivery/administration regimen, respectively (for example route of delivery/administration, dosage regimen, time of delivery/administration, frequency of delivery/administration). In particular, the instruction manual/leaflet may comprise the instruction that the mRNA, respectively, is to be injected and/or is prepared for injection into skeletal muscle. The instruction manual/leaflet may further comprise the instruction that the mRNA, respectively, is prepared for administration during the inflammatory phase which follows the skeletal muscle injury. In principle, what has been said herein elsewhere with respect to the mode of delivery/administration and delivery/administration regimen, respectively, may be comprised as respective instructions in the instruction manual/leaflet.

### EXAMPLES

### Example 1

### Methods and Material

### Cloning of IGF1 and Exchange of Signaling Peptides

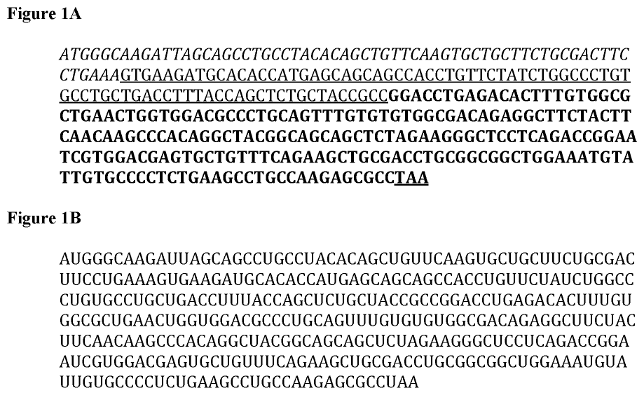

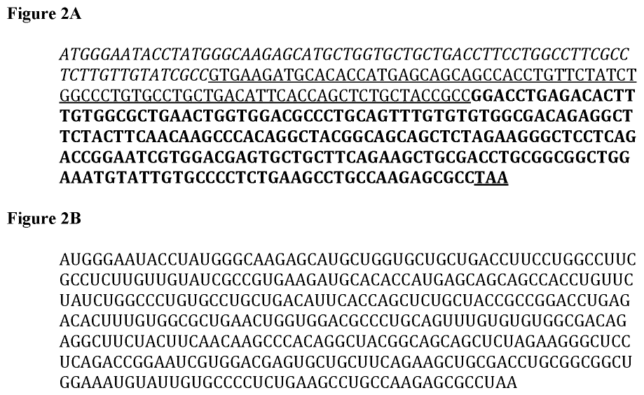

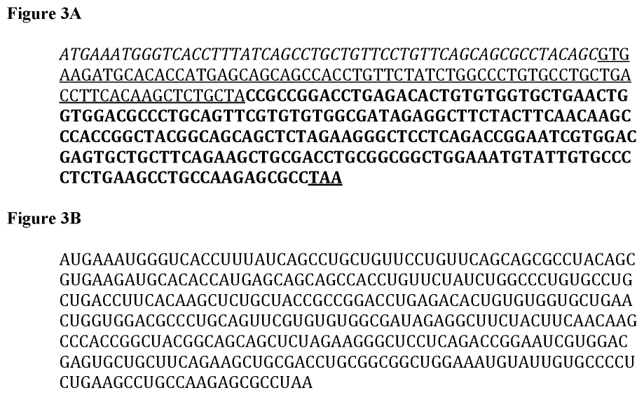

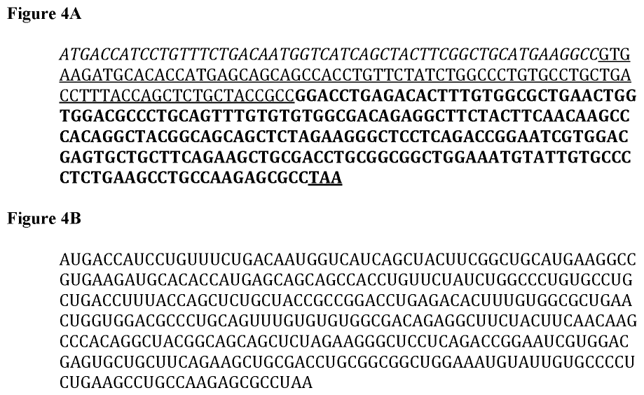

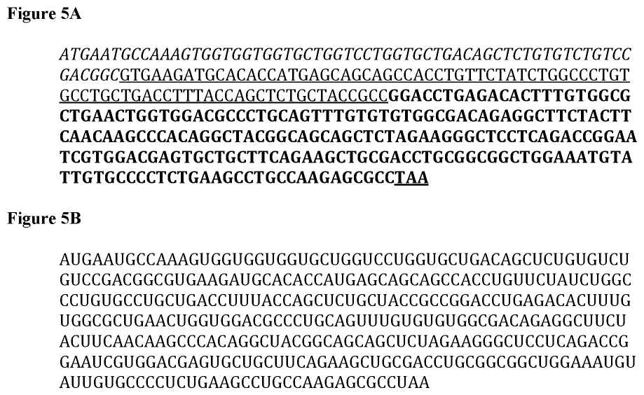

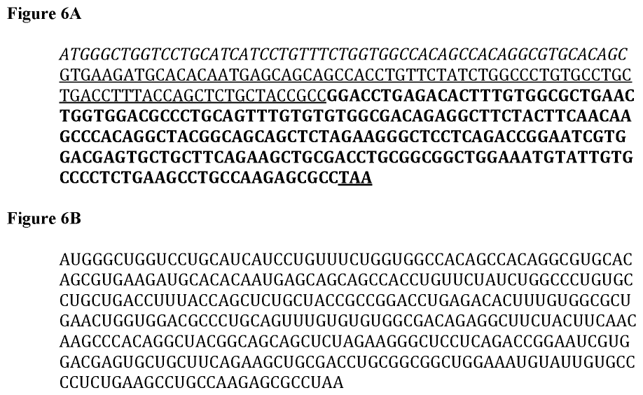

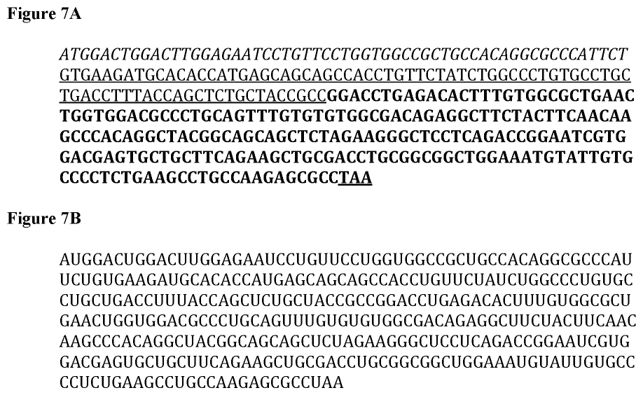

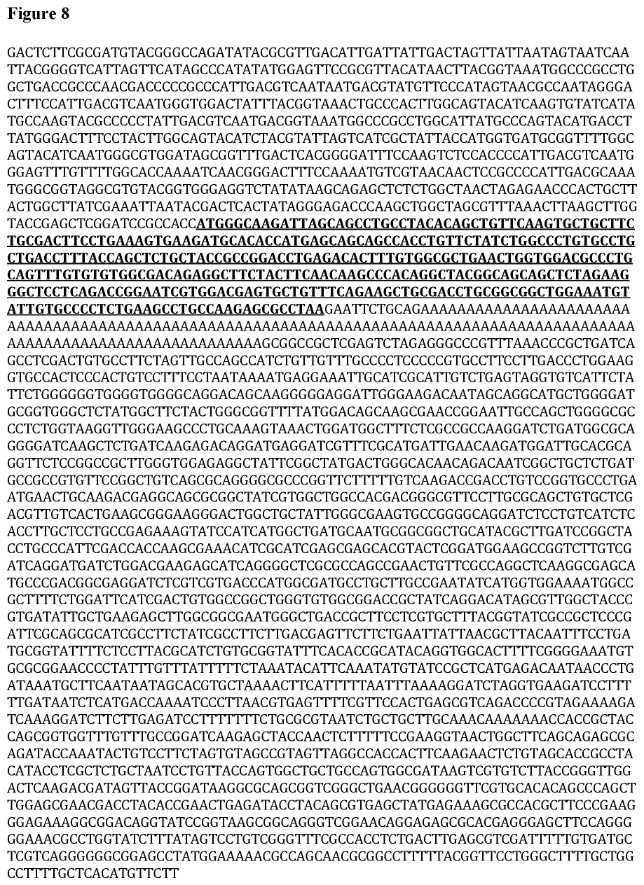

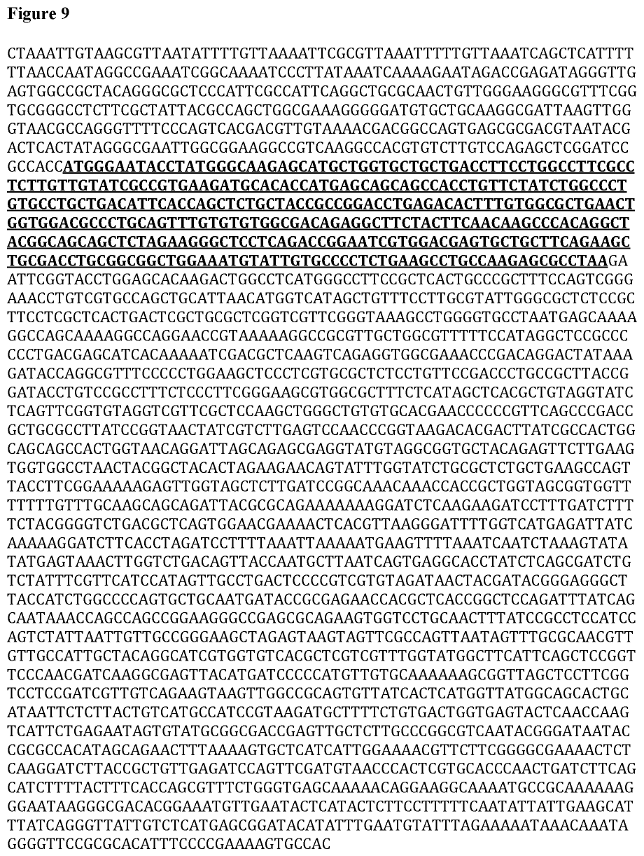

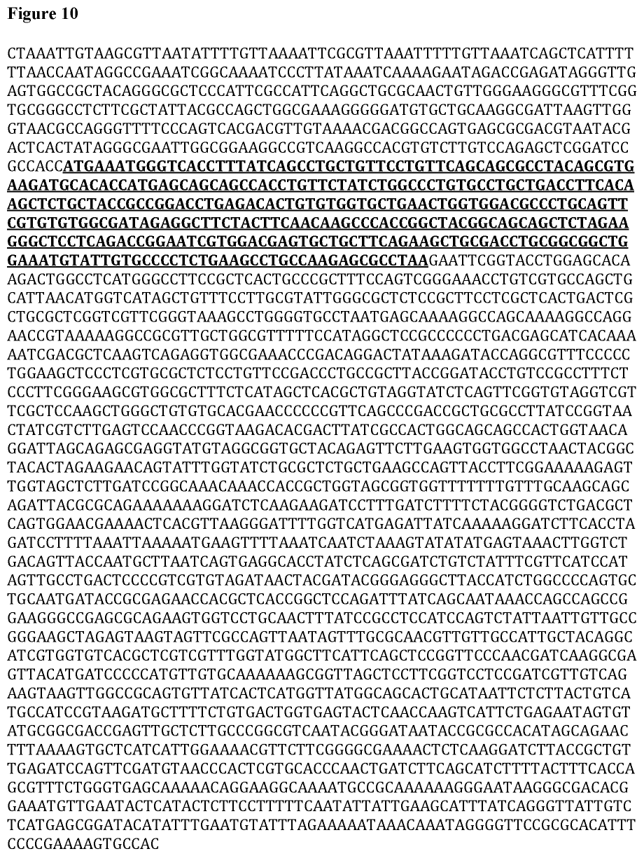

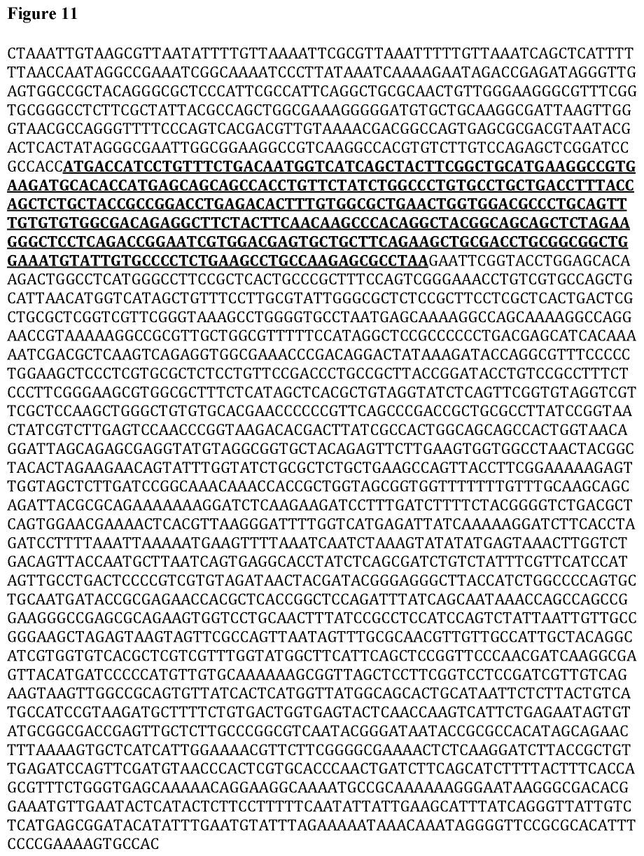

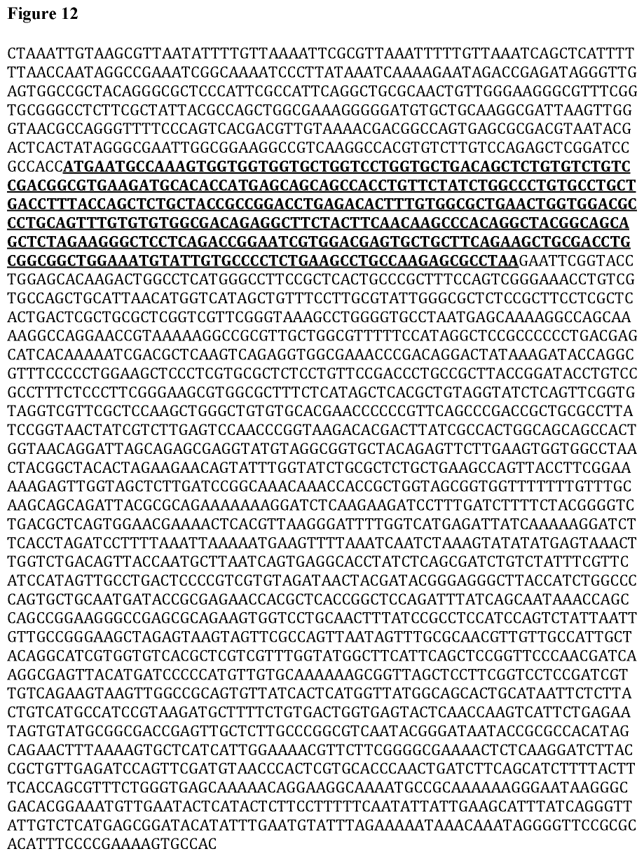

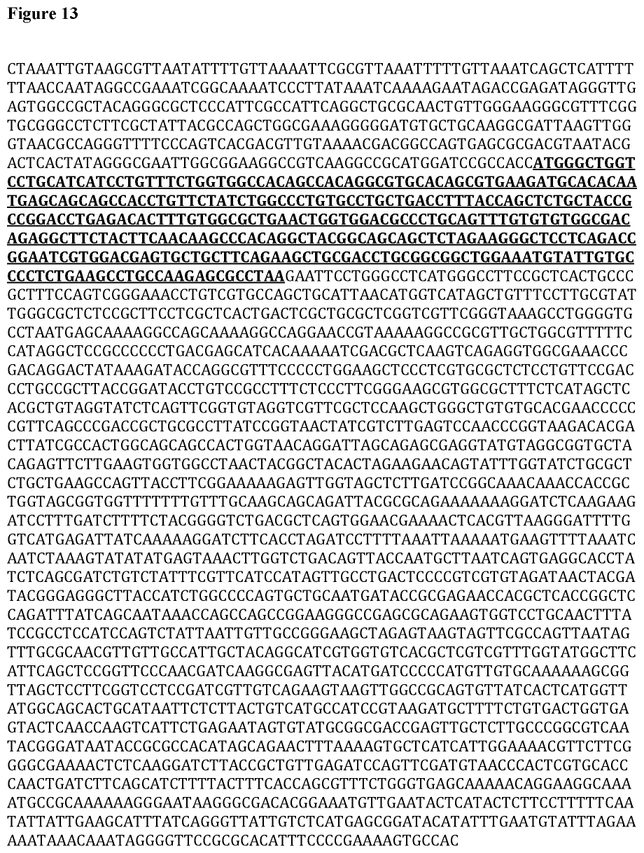

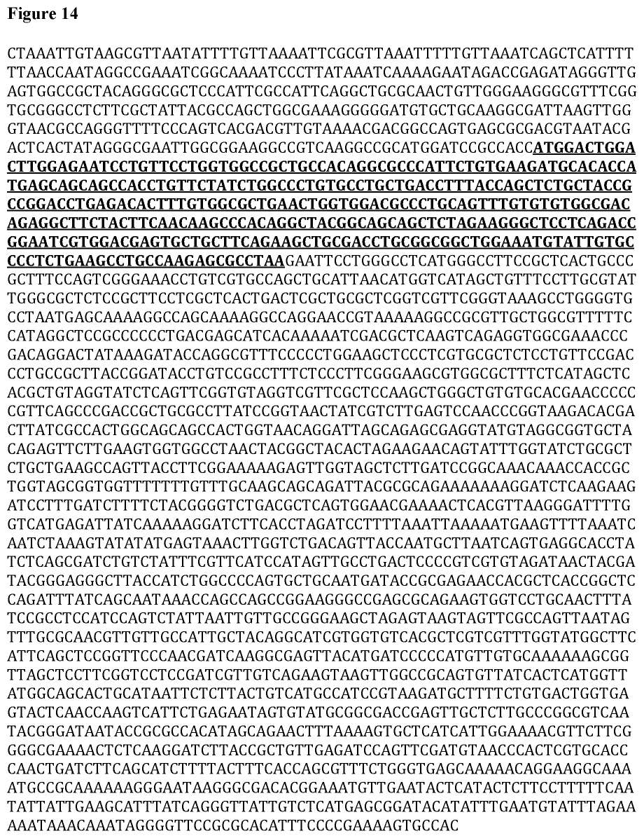

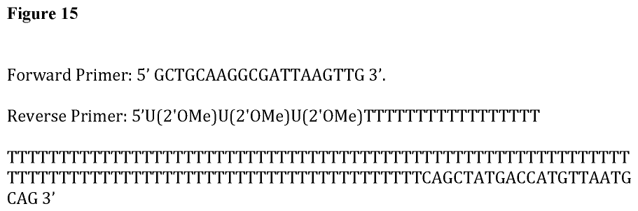

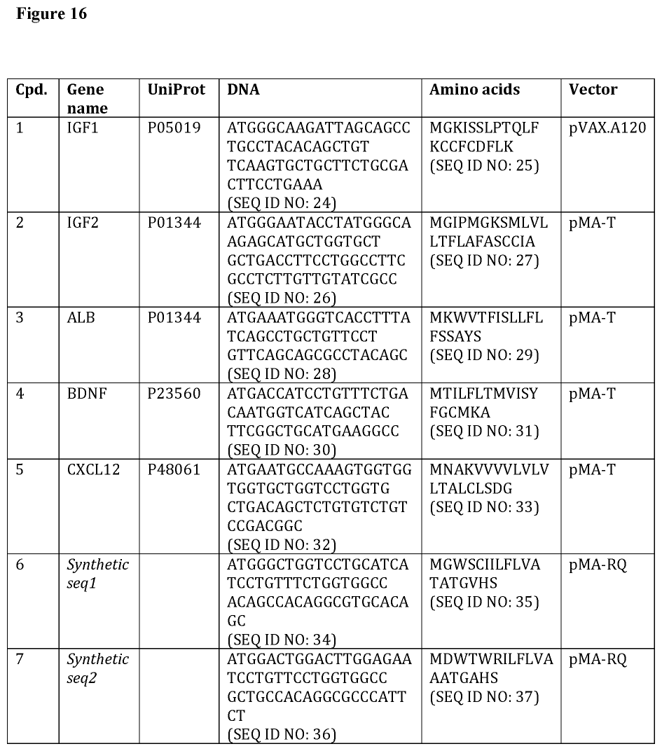

IGF1 is a 70 amino acid polypeptide synthesised in the endoplasmatic reticulum and secreted via the Golgi apparatus to act as extracellular growth factor in an auto- and paracrine manner. For ensuring proper expression and secretion of mRNA-induced IGF1 out of the transfected cell, the mRNA sequence included the natural N-terminal pre-pro-sequence of human IGF1 (pre-pro-IGF1). This sequence consisted of the sequence encoding the pre-domain (signalling peptide) of human IGF1 with 21 amino acids (nucleotides 1-63) and the sequence encoding the human pro-domain with 27 amino acids (nucleotides 64-144). Furthermore, the construct contained the sequence encoding the full coding sequence of mature human IGF1 with 70 amino acids (nucleotides 145-354). In Cpd.2-7, the pre-domain (signaling peptide, nucleotide 1-63) was exchanged by respective pre-domains of IGF2, ALB, BDNF, CXCL12, or the synthetic signalling peptides 1 or 2. No C-terminal E-domain was added to the construct. In summary, the cloning vector contained a copy of the human pre-pro-IGF1 DNA without E-peptide information and was defined as Cpd.1, whereas Cpds.2-7 contained alternative pre-domains (signalling peptides). FIGS. 1A-1B illustrate the DNA and RNA sequence of IGF1 encoded by its pre-, pro and coding domain. FIGS. 2A-2B illustrate the DNA and RNA sequence of IGF1 encoded by IGF2 pre-domain and its pro and coding domain. FIGS. 3A-3B illustrate the DNA and RNA sequence of IGF1 encoded by ALB pre-domain and its pro and coding domain. FIGS. 4A-4B illustrate the DNA and RNA sequence of IGF1 encoded by BDNF pre-domain and its pro and coding domain. FIGS. 5A-5B illustrate the DNA and RNA sequence of IGF1 encoded by CXCL12 pre-domain and its pro and coding domain. FIGS. 6A-6B illustrate the DNA and RNA sequence of IGF1 encoded by synthetic signaling peptide 1 pre-domain and its pro and coding domain. FIGS. 7A-7B illustrate the DNA and RNA sequence of IGF1 encoded by synthetic signaling peptide 2 pre-domain and its pro and coding domain. FIG. 8 illustrates the pVAX.A120 vector (www.thermofisher.com) with the Cpd.1 insert. FIG. 9 illustrates the pMA-T vector (www.thermofisher.com) with the Cpd.2 insert. FIG. 10 illustrates the pMA-T vector with the Cpd.3 insert. FIG. 11 illustrates the pMA-T vector with the Cpd.4 insert. FIG. 12 illustrates the pMA-T vector with the Cpd.5 insert. FIG. 13 illustrates the pMA-RQ vector (www.thermofisher.com) with the Cpd.6 insert. FIG. 14 illustrates the pMA-RQ vector (www.thermofisher.com) with the Cpd.6 insert. FIG. 15 shows the primers that were utilized for amplifying Cpd.2-7. FIG. 16 summarises the identities of the different pre-domains by indicating the gene name, the UniProt number, the DNA and the amino acid sequence of the pre-domains and the vectors. For Cpd.6 and Cpd.7 no gene name exists as they were artificial pre-domains. Codon optimization of the DNA and mRNA sequences of Cpds.1-7 where done by using GeneOptimizer® (ThermoFischer, MA).

The open reading frame of the pre-pro-IGF1 DNA sequences was synthesized from GeneArt (www.thermofisher.com, ThermoFischer, MA) with BamHI and EcoRI restriction sites and was sub-cloned into pVAX1.A120 vector using the same restriction enzymes. The DNA sequence of the entire vector are given in FIG. 8. Orientation of the cloned inserts and base sequences were confirmed by Sanger sequencing of several clones. The successful clone was selected as template for the in vitro transcription (IVT) mRNA production. For the alternative pre-domain variants Cpd.2-Cpd.7, the pMA-T (FIGS. 9-12) and pMA-RQ (FIGS. 13-14) vectors were used as templates for the IVT. All IVT reactions resulted in mRNAs with identical polyA120 tails.

Exchange of Signaling Peptides in Cpd.8 to Cpd.39 mRNA

In Cpd.8-26 and Cpd.39, the pre-domain (signaling peptide, nucleotide 1-63) of IGF1 (i.e., Cpd.1) was exchanged by respective pre-domains of LTBP2 (Cpd. 8; Uniprot ID: Q14767), IGFALS (Cpd. 9; Uniprot ID: P35858), INS (Cpd.10; Uniprot ID: P01308),), Epo (Cpd.11; Uniprot ID: P01588), CSF3 (Cpd.12; Uniprot ID: P09919), NGF (Cpd.13; Uniprot ID: P01138), FGF5 (Cpd.14; Uniprot ID: P12034), FHR2 (Cpd.15; Uniprot ID: P36980), IBP5 (Cpd.16; Uniprot ID: P24593), NTF3 (Cpd.17; Uniprot ID: P20783), PATE2 (Cpd.18; Uniprot ID: Q6UY27), SOD3 (Cpd.19; Uniprot ID: P08294), part of coding sequence of GLR (Cpd. 20; Uniprot ID: P47871), modified pre-domain sequence of IGF1 (Cpd. 21; Uniprot ID: P05019), modified pre-domain sequence of IGF2 (Cpd. 22; Uniprot ID: P01344), modified pre-domain sequence of CXCL12 (Cpd. 23; Uniprot ID: P48061), modified pre-domain sequence of BDNF (Cpd. 24; Uniprot ID: P23560), modified pro-domain sequence of IGF1 (Cpd. 25; Uniprot ID: P05019), modified pro-domain sequence of ALPI (Cpd. 39; Uniprot ID: P09923) and modified pre-domain sequence of INS (Cpd. 26; Uniprot ID: P01308). As similar with Cpd.1, all above specified compounds were without E-peptide. Codon optimization of the DNA and mRNA sequences of Cpds.8-26 and Cpd. 39 where done by using GeneOptimizer® (ThermoFischer, MA).

The Cpd.27 consisted of the sequence encoding the pre-domain (signalling peptide) of human erythropoietin (Epo; Uniprot ID: P01588) with 27 amino acids (nucleotides 1-81) and the sequence encoding the coding chain for human erythropoietin with 166 amino acids (nucleotides 82-498). In Cpd.28 and Cpd.29, the pre-domain (signaling peptide, nucleotide 1-81) of Epo was exchanged by modified pre-domain sequence of Epo (Uniprot ID: P01588) and pre-domain sequence of BDNF (Uniprot ID: P23560). Codon optimization of the DNA and mRNA sequences of Cpds.27-29 where done by using GeneOptimizer® (ThermoFischer, MA).

The Cpd.30 consisted of the sequence encoding the pre-domain (signalling peptide) of human insulin (INS; Uniprot ID: P01308) with 24 amino acids (nucleotides 1-72), and the sequence encoding B-chain domain with 30 amino acids (nucleotides 73-162), and the sequence encoding connecting peptide (C-peptide) domain with 31 amino acids (nucleotides 163-255), and the sequence encoding A-chain domain with 21 amino acids (nucleotides 256-330). In Cpd.31 and Cpd.32, the pre-domain (signaling peptide, nucleotide 1-72) of INS was exchanged by modified pre-domain sequence of INS (Uniprot ID: P01308) and pre-domain sequence of BDNF (Uniprot ID: P23560). Codon optimization of the DNA and mRNA sequences of Cpds. 30-32 where done by using GeneOptimizer® (ThermoFischer, MA).

The Cpd.33 consisted of the sequence encoding the pre-domain (signalling peptide) of human interleukin 4 (IL-4; Uniprot ID: P05112) with 24 amino acids (nucleotides 1-72), and the sequence encoding the coding chain domain with 129 amino acids (nucleotides 73-387). In Cpd.34 and Cpd.35, the pre-domain (signaling peptide, nucleotide 1-72) of IL-4 was exchanged by modified pre-domain sequence of IL-4 (Uniprot ID: P05112) and pre-domain sequence of FGF5 (Uniprot ID: P01308). Codon optimization of the DNA and mRNA sequences of Cpds.33-35 where done by using GeneOptimizer® (ThermoFischer, MA).

The Cpd.36 consisted of the sequence encoding the pre-domain (signalling peptide) of human interleukin 10 (IL-10; Uniprot ID: P22301) with 24 amino acids (nucleotides 1-54), and the sequence encoding the coding chain domain with 160 amino acids (nucleotides 55-534). In Cpd.37 and Cpd.38, the pre-domain (signaling peptide, nucleotide 1-54) of IL-10 was exchanged by modified pre-domain sequence of IL-10 (Uniprot ID: P22301) and pre-domain sequence of BDNF (Uniprot ID: P23560). Codon optimization of the DNA and mRNA sequences of Cpds. 36-38 where done by using GeneOptimizer® (ThermoFischer, MA).

The amino acid sequence and DNA sequence of signal peptides of Cpd.1-39 and RNA sequence and DNA sequence and vector of respective Cpd.1-39 are displayed in Table 1 below.

TABLE 1
Amino acid sequence and DNA sequence of signal peptides of Cpd. 1-39 and RNA
sequence and DNA sequence and vector of Cpd. 1-39

| | | | | | DNA | RNA | DNA | |
|---|---|---|---|---|---|---|---|---|
| | | | | SP | seq. | seq. | seq. of | |
| Cpd | Signalling | | SP AA | DNA | of | of | vector | |
| No. | peptide (SP) | Protein | seq. | seq. | Cpd. | Cpd. | of Cpd. | Vector |
| 1 | IGF1 | IGF1 | SEQ ID | SEQ ID | SEQ ID | SEQ ID | SEQ ID | pVAX.A |
| | | | NO: 25 | NO: 24 | NO: 1 | NO: 2 | NO: 15/ | 120/pM |
| | | | | | | | SEQ ID | A-T |
| | | | | | | | NO: 40 | |
| 2 | IGF2 | IGF1 | SEQ ID | SEQ ID | SEQ ID | SEQ ID | SEQ ID | pMA-T |
| | | | NO: 27 | NO: 26 | NO: 3 | NO: 4 | NO: 16 | |
| 3 | ALB | IGF1 | SEQ ID | SEQ ID | SEQ ID | SEQ ID | SEQ ID | pMA-T |
| | | | NO: 29 | NO: 28 | NO: 5 | NO: 6 | NO: 17 | |
| 4 | BDNF | IGF1 | SEQ ID | SEQ ID | SEQ ID | SEQ ID | SEQ ID | pMA-T |
| | | | NO: 31 | NO: 30 | NO: 7 | NO: 8 | NO: 18 | |
| 5 | CXCL12 | IGF1 | SEQ ID | SEQ ID | SEQ ID | SEQ ID | SEQ ID | pMA-T |
| | | | NO: 33 | NO: 32 | NO: 9 | NO: 10 | NO: 19 | |
| 6 | Synthetic | IGF1 | SEQ ID | SEQ ID | SEQ ID | SEQ ID | SEQ ID | PMA- |
| | Seq1 | | NO: 35 | NO: 34 | NO: 11 | NO: 12 | NO: 20 | RQ |
| 7 | Synthetic | IGF1 | SEQ ID | SEQ ID | SEQ ID | SEQ ID | SEQ ID | PMA- |
| | seq2 | | NO: 37 | NO: 36 | NO: 13 | NO: 14 | NO: 21 | RQ |
| 8 | LTBP2 | IGF1 | SEQ ID | SEQ ID | SEQ ID | SEQ ID | SEQ ID | PMA- |
| | | | NO: 41 | NO: 42 | NO: 43 | NO: 44 | NO: 45 | RQ |
| 9 | IGFALS | IGF1 | SEQ ID | SEQ ID | SEQ ID | SEQ ID | SEQ ID | PMA- |
| | | | NO: 46 | NO: 47 | NO: 48 | NO: 49 | NO: 50 | RQ |
| 10 | INS | IGF1 | SEQ ID | SEQ ID | SEQ ID | SEQ ID | SEQ ID | PMA- |
| | | | NO: 51 | NO: 52 | NO: 53 | NO: 54 | NO: 55 | RQ |
| 11 | EPO | IGF1 | SEQ ID | SEQ ID | SEQ ID | SEQ ID | SEQ ID | pMA- |
| | | | NO: 56 | NO: 57 | NO: 58 | NO: 59 | NO: 60 | RQ |
| 12 | CSF3 | IGF1 | SEQ ID | SEQ ID | SEQ ID | SEQ ID | SEQ ID | pMA- |
| | | | NO: 61 | NO: 62 | NO: 63 | NO: 64 | NO: 65 | RQ |
| 13 | NGF | IGF1 | SEQ ID | SEQ ID | SEQ ID | SEQ ID | SEQ ID | pMA- |
| | | | NO: 66 | NO: 67 | NO: 68 | NO: 69 | NO: 70 | RQ |
| 30 | INS | Insulin | SEQ ID | SEQ ID | SEQ ID | SEQ ID | SEQ ID | pMA- |
| | | | NO: 51 | NO: 52 | NO: 71 | NO: 72 | NO: 73 | RQ |
| 27 | EPO | EPO | SEQ ID | SEQ ID | SEQ ID | SEQ ID | SEQ ID | pMA- |
| | | | NO: 56 | NO: 57 | NO: 74 | NO: 75 | NO: 76 | RQ |
| 33 | IL4 | IL4 | SEQ ID | SEQ ID | SEQ ID | SEQ ID | SEQ ID | pMA- |
| | | | NO: 77 | NO: 78 | NO: 79 | NO: 80 | NO: 81 | RQ |
| 36 | IL10 | IL10 | SEQ ID | SEQ ID | SEQ ID | SEQ ID | SEQ ID | pMA- |
| | | | NO: 81 | NO: 83 | NO: 84 | NO: 85 | NO: 86 | RQ |
| 14 | FGF5 | IGF1 | SEQ ID | SEQ ID | SEQ ID | SEQ ID | SEQ ID | pMA- |
| | | | NO: 87 | NO: 88 | NO: 89 | NO: 90 | NO: 91 | RQ |
| 15 | FHR2 | IGF1 | SEQ ID | SEQ ID | SEQ ID | SEQ ID | SEQ ID | pMA-T |
| | | | NO: 92 | NO: 93 | NO: 94 | NO: 95 | NO: 96 | |
| 16 | IBP5 | IGF1 | SEQ ID | SEQ ID | SEQ ID | SEQ ID | SEQ ID | pMA- |
| | | | NO: 97 | NO: 98 | NO: 99 | NO: 100 | NO: 101 | RQ |
| 17 | NTF3 | IGF1 | SEQ ID | SEQ ID | SEQ ID | SEQ ID | SEQ ID | pMA- |
| | | | NO: | NO: | NO: | NO: | NO: 106 | RQ |
| | | | 102 | 103 | 104 | 105 | | |
| 18 | PATE2 | IGF1 | SEQ ID | SEQ ID | SEQ ID | SEQ ID | SEQ ID | pMA-T |
| | | | NO: | NO: | NO: | NO: | NO: 111 | |
| | | | 107 | 108 | 109 | 110 | | |
| 19 | SOD3 | IGF1 | SEQ ID | SEQ ID | SEQ ID | SEQ ID | SEQ ID | pMA- |
| | | | NO: | NO: | NO: | NO: | NO: 116 | RQ |
| | | | 112 | 113 | 114 | 115 | | |
| 20 | GLR | IGF1 | SEQ ID | SEQ ID | SEQ ID | SEQ ID | SEQ ID | pMA-T |
| | | | NO: | NO: | NO: | NO: | NO: 121 | |
| | | | 117 | 118 | 119 | 120 | | |
| 21 | IGF1- | IGF1 | SEQ ID | SEQ ID | SEQ ID | SEQ ID | SEQ ID | pMA-T |
| | Modified | | NO: | NO: | NO: | NO: | NO: 126 | |
| | | | 122 | 123 | 124 | 125 | | |
| 22 | IGF2- | IGF1 | SEQ ID | SEQ ID | SEQ ID | SEQ ID | SEQ ID | pMA-T |
| | Modified | | NO: | NO: | NO: | NO: | NO: 131 | |
| | | | 127 | 128 | 129 | 130 | | |
| 23 | CXCL12- | IGF1 | SEQ ID | SEQ ID | SEQ ID | SEQ ID | SEQ ID | pMA-T |
| | Modified | | NO: | NO: | NO: | NO: | NO: 136 | |
| | | | 132 | 133 | 134 | 135 | | |
| 24 | BDNF- | IGF1 | SEQ ID | SEQ ID | SEQ ID | SEQ ID | SEQ ID | pMA-T |
| | Modified | | NO: | NO: | NO: | NO: | NO: 141 | |
| | | | 137 | 138 | 139 | 140 | | |
| 25 | IGF1- | IGF1 | SEQ ID | SEQ ID | SEQ ID | SEQ ID | SEQ ID | pMA-T |
| | Pro- | | NO: | NO: | NO: | NO: | NO: 146 | |
| | Modified | | 142 | 143 | 144 | 145 | | |
| 26 | INS- | IGF1 | SEQ ID | SEQ ID | SEQ ID | SEQ ID | SEQ ID | pMA-T |
| | Modified | | NO: | NO: | NO: | NO: | NO: 151 | |
| | | | 147 | 148 | 149 | 150 | | |
| 28 | Epo- | EPO | SEQ ID | SEQ ID | SEQ ID | SEQ ID | SEQ ID | pMA-T |
| | Modified | | NO: | NO: | NO: | NO: | NO: 156 | |
| | | | 152 | 153 | 154 | 155 | | |
| 29 | BDNF | EPO | SEQ ID | SEQ ID | SEQ ID | SEQ ID | SEQ ID | pMA- |
| | | | NO: 31 | NO: 30 | NO: | NO: | NO: 159 | RQ |
| | | | | | 157 | 158 | | |
| 31 | INS- | INS | SEQ ID | SEQ ID | SEQ ID | SEQ ID | SEQ ID | pMA-T |
| | Modified | | NO: | NO: | NO: | NO: | NO: 162 | |
| | | | 147 | 182 | 160 | 161 | | |
| 32 | BDNF | INS | SEQ ID | SEQ ID | SEQ ID | SEQ ID | SEQ ID | pMA- |
| | | | NO: 31 | NO: 30 | NO: | NO: | NO: 165 | RQ |
| | | | | | 163 | 164 | | |
| 34 | IL4- | IL4 | SEQ ID | SEQ ID | SEQ ID | SEQ ID | SEQ ID | pMA-T |
| | Modified | | NO: | NO: | NO: | NO: | NO: 170 | |
| | | | 166 | 167 | 168 | 169 | | |
| 35 | FGF5 | IL4 | SEQ ID | SEQ ID | SEQ ID | SEQ ID | SEQ ID | pMA- |
| | | | NO: 87 | NO: | NO: | NO: | NO: 173 | RQ |
| | | | | 183 | 171 | 172 | | |
| 37 | IL10- | IL10 | SEQ ID | SEQ ID | SEQ ID | SEQ ID | SEQ ID | pMA-T |
| | Modified | | NO: | NO: | NO: | NO: | NO: 178 | |
| | | | 174 | 175 | 176 | 177 | | |
| 38 | BDNF | IL10 | SEQ ID | SEQ ID | SEQ ID | SEQ ID | SEQ ID | pMA- |
| | | | NO: 31 | NO: 30 | NO: | NO: | NO: 181 | RQ |
| | | | | | 179 | 180 | | |
| 39 | ALPI- | IGF1 | SEQ ID | SEQ ID | SEQ ID | SEQ ID | SEQ ID | pMA-T |
| | Pro- | | NO: | NO: | NO: | NO: | NO: 193 | |
| | Modified | | 189 | 190 | 191 | 192 | | |

In Vitro Transcription (IVT) of Cpd.1 to Cpd.7 mRNA

The pVAX.A120 vector containing Cpd.1 (SEQ ID No. 15) also possessed a T7 promoter and a poly-A tail of 120 bp length, and the vector was linearized downstream of the poly-A tail with XhoI enzyme prior to mRNA production using in vitro transcription (IVT). For the pMA-T and pMA-RQ vectors, a homologous primer pair (SEQ ID Nos: 22 and 23) was used for PCR based IVT-mRNA production (FIG. 15). The reverse primer contained 120 bp poly-A to include a poly-A tail into the mature mRNA. Both linearized plasmids and PCR amplicons were used as templates for IVT performed by T7 RNA polymerase in the MEGAscript T7 kit (www.ambion.com). All mRNAs were produced with an anti-reverse CAP analog (ARCA; [m7G(5′)G]) in the 5′ end and chemically modified with 100% N1-methylpseudo-UTP (www.trilink.com). In vitro transcribed mRNAs were purified using the MEGAclear kit (www.ambion.com) and analyzed for quality and concentration using RNA 6000 Nano kit in an Agilent 2100 Bioanalyzer (www.agilent.com).

In vitro transcription (IVT) of Cpd.1 and Cpd.8 to Cpd.39 mRNA For the pMA-T and pMA-RQ vectors encoding Cpd.1 (SEQ ID No. 40; prior to sub-cloning into pVAX.A120 vector) and Cpd. 8 to Cpd.39, a homologous primer pair (SEQ ID Nos: 22 and 23) was used for PCR based IVT-mRNA production (FIG. 15). The reverse primer contained 120 bp poly-A to include a poly-A tail into the mature mRNA. The PCR amplicons were used as templates for IVT performed by T7 RNA polymerase in the MEGAscript T7 kit (www.ambion.com). All mRNAs were produced with an anti-reverse CAP analog (ARCA; [m7G(5′)G]) in the 5′ end and chemically modified with 100% N1-methylpseudo-UTP (www.trilink.com). In vitro transcribed mRNAs were purified using the MEGAclear kit (www.ambion.com) and analyzed for quality and concentration using RNA agarose gel electrophoresis.

### In Vitro Transfection of HEK293T, C2C12 and HepG2 Cells

Human embryonic kidney cells 293 (HEK293T; ATCC, CRL-1573, Rockville, Md., USA) was maintained in Dulbecco's Modified Eagle's medium (DMEM, www.biochrom.com) supplemented with 10% (v/v) Fetal Bovine Serum (FBS), and Penicillin-Streptomycin-Amphotericin B mixture (882087, Biozym, Oldendorf, Germany). Cells were seeded at 7,000-20,000 cell/well in a 96 well culture plate and incubated at 37° C. in a humidified atmosphere containing 5% CO₂ for 24 hours prior to transfection. Cells were grown in DMEM growth medium containing 10% of FBS without antibiotics to reach confluency <60% before transfection.

Human hepatoma cell line HepG2 (Cat #85011430, ECACC UK) was grown in Dulbecco's Modified Eagle's medium (DMEM) with 10% fetal calf serum and Penicillin-Streptomycin-Amphotericin B mixture (882087, Biozym, Oldendorf, Germany) at 37° C. in a humidified atmosphere containing 5% CO₂. HepG2 cells were sub-cultured every 2 and every 5 days at a splitting ratio of 1:2 and 1:4, respectively. Cells were plated at a density of 20,000-40,000 cells/well 24 hours prior to transfection in a 96-well-microtiter plate. Cells were grown in DMEM growth medium containing 10% of FBS without antibiotics to reach 30-40% confluency before transfection.

Mouse myoblast cell line C2C12 (ATCC, CRL-1772, Rockville, Md., USA) were grown in Dulbecco's Modified Eagle's medium (DMEM) with 10% fetal calf serum and Penicillin-Streptomycin-Amphotericin B mixture (882087, Biozym, Oldendorf, Germany) at 37° C. in a humidified atmosphere containing 5% CO₂. C2C12 cells were sub-cultured every 2 and every 5 days at a splitting ratio of 1:2 and 1:4, respectively. Cells were plated at a density of 20,000 cells/well 24 hours prior to transfection in a 96-well-microtiter plate. Cells were grown in DMEM growth medium containing 2% of FBS without antibiotics to reach 80-90% confluency before transfection.

Thereafter, cells were transfected with different mRNA variants at 0.3 μs using Lipofectamine 2000 (www.invitrogen.com) following the manufacturer's instructions. The 100 μl of DMEM was removed and replaced by 50 μl of Opti-MEM and 50 μl mRNA and Lipofectamine 2000 complex in Opti-MEM (www.thermofisher.com). After 5 hours, the medium was replaced by fresh medium and the plates were incubated 24 hours at 37° C. in a humidified atmosphere containing 5% CO₂.

### In Vitro Transfection of HSkMC Cells

HSkMC cells were plated at in a density of 40.000 cells per 96-well on a microtiter-plate in SkM growth medium (PromoCell, Heidelberg, Germany). Cells were grown for 1 day in a humidified atmosphere at 37° C. incubator and 5% CO₂ to a confluence of >90%. On the day of transfection cells were treated with different mRNA variants (Cpd.1 or 4) at 2 μg using Lipofectamin 2000 (www.invitrogen.com). Therefore, 100 μl medium were removed, and 1 μl Lipofectamin/well added together with 2 μg mRNA/well in OPTIMEM medium (www.thermofisher.com). The cells were then incubated in a humidified atmosphere at 37° C. and 5% CO₂ for 24 hours.

### In Vitro Transfection of IMR32 Cells

24 hours prior transfection, Human Caucasian Neuroblastoma IMR32 cells (Cat #86041809, ECACC, UK) were plated at a density 60,000 cells per well in a 96 pre coated BRAND microtiter plate (Cat #782082) in Minimum Essential Medium Eagle (EMEM, Bioconcept Cat #1-31S01-I, www.bioconcept.ch) supplemented with 10% (v/v) heat-inactivated Fetal Bovine Serum (FBS), L-Glutamine (2 mM) and Non-essential Amino acids (NEAA, 1×). Cells were grown overnight at 37° C. in a humidified atmosphere containing 5% CO₂. Cells were transfected with 0.3 μg of mRNA-constructs using JetMessenger (www.polyplus-transfection.com) following manufacturer's instructions. Briefly mRNA/JetMessenger complex was formed by mixing 0.25 μl JetMessenger reagent per 0.1 μg mRNA construct. After incubating 15 minutes at room temperature the JetMessenger complex was added as 10 μl and 5 hours after transfection medium/mRNA/JetMessenger was removed from the wells and replaced with fresh 100 μl growth medium and the plates were incubated 24 hours at 37° C. in a humidified atmosphere containing 5% CO₂.

### In Vitro Transfection of A549 Cells

Human Lung carcinoma cell line (Sigma-Aldrich, Buchs Switzerland cat #6012804) was maintained on Dulbecco's Modified Eagle's medium-high glucose (DMEM, Sigma-Aldrich, Buchs Switzerland cat #D0822) supplemented with 10% FBS (Thermofischer, Basel, Switzerland cat #10500-064). 24 hours prior transfection the A549 cell were plated at a density of 10,000 cells/well in a regular growth medium. Thereafter, cells were transfected with different mRNAs (0.3-0.6 μg) using Lipofectamine 2000 (www.invitrogen.com) following the manufacturer's instructions. 100 μl of DMEM was removed. 50 μl of Opti-MEM (www.thermofisher.com) was added to each well followed by 50 μl mRNA and Lipofectamine 2000 complex in Opti-MEM. After 5 hours of incubation, the medium was replaced by fresh growth medium and the plates were incubated 24 hours at 37° C. in a humidified atmosphere containing 5% CO₂.

### In Vitro Transfection of THP-1 Cells

Human monocyte leukemia cell line THP-1 (Sigma-Aldrich, Buchs Switzerland, Cat. #88081201) was maintained in growth medium (RPMI 1640 supplemented with 10% FBS and 2 mM glutamine. The cells were seeded at 30,000 THP-1 cells in a 96 well cell culture plate 72h before transfection and activated with 50 nM of phorbol 12-myristate 13-acetate (PMA) (Sigma-Aldrich, Buchs Switzerland, Cat. #P8139) diluted in growth medium. The cells were transfected with (300-600 ng/well) of mRNA using Lipofectamine 2000 (www.thermofisher.com) 100 μl of DMEM was removed. 50 μl of Opti-MEM (www.thermofisher.com) was added to each well followed by 50 μl mRNA and Lipofectamine 2000 complex in Opti-MEM. After 5 hours, the medium was replaced by fresh growth medium supplemented with 50 nM PMA and the plates were incubated 24 hours at 37° C. in a humidified atmosphere containing 5% CO₂.

### Rat Primary Spinal Cord Neurons

Pregnant female wild type Wistar rats (Janvier labs, France) or SOD1G93A Sprague Dawley rats (Taconic Bioscience) of 14 days in gestation were sacrificed using deep anesthesia with CO₂ and cervical dislocation. The fetuses were removed from the uterus and immediately placed in ice cold Leibovitz medium supplemented with 2% penicillin (10,000 U/mL) and streptomycin (10 mg/mL) solution (PS) and 1% bovine serum albumin (BSA). Spinal cords were dissected and treated for 20 minutes at 37° C. with a 0.05% trypsin-0.02% EDTA. The dissociation was stopped by the addition of Dulbecco's modified Eagle's medium (DMEM) supplemented with 4.5 g/l of glucose, 0.5 mg/mL DNAase I and II, and 10% fetal calf serum (FCS). Cells were mechanically dissociated by three forced passages through the tip of a 10 mL pipette. Further, cells were centrifuged at 515 g for 10 minutes at 4° C. The resulting pellet were resuspended in a defined culture medium consisting of neurobasal medium containing a 2% solution of B27 supplement, 2 mmol/1 of glutamine, 2% of PS solution and 10 ng/ml of brain derived neurotrophic factor (BDNF). Cells were seeded at 20,000 cells pro well in a 96 well poly-D-Lysine precoated plate and cultured at 37° C. in a humidified atmosphere containing 5% CO₂. The medium was replaced every two days. After 11-12 days in culture, the mRNA constructs (0.3 μg) were transfected using JetMessenger (www.polyplus-transfection.com) following manufacturer's instructions.

### In Vitro Transfection of Human Differentiated Chondrocytes.

Human articular cartilage chondrocytes (Sigma/Cell Applications, Buchs, Switzerland Cat. #402-05A) were maintained in Chondrocyte growth medium (Sigma Aldrich, Buchs, Switzerland Cat #411-500). Cells were incubated at 37° C. in a humidified atmosphere containing 5% CO₂. Cells were differentiated by growing them for a minimum of 3 weeks on Alginate beads in Differentiation medium (Sigma Aldrich, Buchs, Switzerland Cat. #A411D-250). For preparation of Alginate beads, 1 ml of 1.2% sterile Alginate solution (1.2% Alginate Sigma Aldrich, Buchs Switzerland Cat. #A-2033 in 0.9% NaCl) were used per 4×10⁶ chondrocytes. Cells were resuspended in the according volume of 1.2% Alginate solution and dropwise dispensed through a 22-gauge needle into 100 mM CaCl₂ solution in a 6-well non-treated cell culture plate. After 15 minutes polymerization beads were washed 5 times with 0.9% NaCl and 2 times with differentiation medium. Chondrocytes/Alginate beads were incubated at 37° C. in a humidified atmosphere containing 5% CO₂ for differentiation for minimum 3 weeks with a medium change every two days. 24 hours prior to transfection differentiated chondrocytes were released from the Alginate beads by washing them 2× with 0.9% NaCl and incubating for about 5 minutes in Alginate dissolving buffer (55 mM Sodium Citrate, 150 mM NaCl, 30 mM EDTA pH 6.8). Cells are washed 2× with 0.9% NaCl. 30,000 cells per well were seeded in 100 μl growth medium in a 96-well TPP plate (Sigma Aldrich, Buchs, Switzerland Cat. #92096) and grown overnight. Cells were transfected with 0.6 μg of mRNA-construct using JetMessenger (www.polyplus-transfection.com) following the manufacturer's instructions. The mRNA/JetMessenger complex was added as 10 μl in quadruplicate. The mRNA/JetMessenger complex was formed by mixing 0.25 μl JetMessenger reagent per 0.1 μg mRNA construct and incubating for 15 minutes at room temperature. After 5 hours of post-transfection, the transfection complex (medium/mRNA/JetMessenger) was removed from the wells and replaced with 100 μl growth medium. The plates were incubated for 24 hours at 37° C. in a humidified atmosphere containing 5% CO₂.

### Analysis of Protein Level in Cell Culture Supernatants

At 24 hours after transfection, the supernatants from the transfected cells were collected, frozen and stored at 20° C. until quantitative analysis of IGF1 (Cat. #E20, Mediagnost, Reutlingen, Germany), erythropoietin (EPO; Cat. #BMS2035, ThermoFisher, Basel, Switzerland), insulin (INS, Cat. #RAB0327, Sigma-Aldrich, Buchs, Switzerland), interleukin 4 (IL-4, Cat. #88-7046-22, ThermoFisher, Basel, Switzerland) and interleukin 10 (IL-10, Cat. #KIT 10947 Sino Biological, China), by ELISA according to the manufacturer ‘instructions. The cell supernatants were analyzed after dilution with the respective ELISA buffer.

### Data Analysis

For the estimation of the proteins (IGF1, EPO, INS, IL-4, IL-10) levels in the standard or the sample, the mean absorbance value of the blank was subtracted from the mean absorbance of the standards or the samples. A standard curve was generated and plotted using a four parameters nonlinear regression according to manufacturer's protocol. To determine the concentration of proteins (IGF1, EPO, INS, IL-4, IL-10) in each sample the concentration of the different protein was interpolated from the standard curve. The final protein concentration of the sample was calculated by multiplication with the dilution factor. All calculations were made using GraphPad Prism 8 (San Diego, USA). For the expression of the fold of increase compared to the endogenous signaling peptide construct the level of protein produced by the individual construct was divided by the level of protein generated by endogenous signaling peptide construct at the same concentration.

### Results

### Cloning of IGF1

Successful cloning of all inserts into pVAX.A120 was confirmed by Sanger sequencing. All tested clones resulted in correct orientation of IGF1 insert with 100% accuracy in sequence. Positive clones were selected for IVT mRNA production.

### Average Hydrophobicity and Polarity of Cpds.1-39

Average hydrophobicity of Cpds.1-39 for amino acids 1-9, for amino acids 1-7 and for amino acids 1-5 of the N-terminal end and for last nine amino acids of the C-terminal end of the amino acid sequence of the signal peptide and average polarity of Cpds.1-39 for amino acids 1-9 of the N-terminal end are as shown in table 2-5 below.

TABLE 2
Average hydrophobicity and polarity of Cpds. 1-39 for amino acids 1-18 of
the N-terminal end and last nine amino acids of the C-terminal end of the
amino acid sequence of the signal peptide and average polarity of Cpds.
1-39 for amino acids 1-9 of the N-terminal end of the signal peptide

| | Hydrophobicity | | | | | | |
|---|---|---|---|---|---|---|---|
| | Score | | | | | | |
| | amino | amino | amino | amino | amino | amino | amino |
| | acids | acids | acids | acids | acids | acids | acids |
| Cpd. No. | 1-9 | 2-10 | 3-11 | 4-12 | 5-13 | 6-14 | 7-15 |
| 1 | 0.614 | 0.114 | 0.071 | 0.129 | 0.029 | 0.543 | 0.1 |
| 2 | 0.344 | 0.556 | 1.067 | 0.989 | 1.589 | 1.3 | 1.656 |
| 3 | 1.211 | 1.422 | 2.167 | 2.689 | 2.533 | 2.522 | 2.122 |
| 5 | 1.878 | 2.133 | 2.944 | 3.211 | 4.067 | 3.522 | 3.256 |
| 6 | 1.989 | 2.2 | 2.711 | 3.011 | 3.022 | 2.944 | 2.367 |
| 7 | 0.278 | 0.489 | 1.344 | 1.644 | 1.922 | 2.222 | 2.644 |
| 8 | −1.867 | −2.256 | −1.8 | −2.122 | −1.422 | −0.922 | −0.989 |
| 9 | 0.433 | 0.644 | 0.644 | 0.644 | 1.567 | 2.422 | 2.889 |
| 10 | 1.111 | 1.322 | 1.544 | 1.322 | 1.844 | 2.056 | 2.756 |
| 11 | 0.089 | 0.3 | 0.244 | 0.2 | 0.978 | 1.789 | 1.422 |
| 12 | −0.344 | −0.344 | −0.978 | −0.511 | −0.122 | −0.122 | 0.378 |
| 13 | 1.767 | 1.478 | 1.767 | 1.867 | 1.867 | 2.056 | 2.156 |
| 30 | 1.111 | 1.322 | 1.544 | 1.322 | 1.844 | 2.056 | 2.756 |
| 27 | 0.089 | 0.3 | 0.244 | 0.2 | 0.978 | 1.789 | 1.422 |
| 33 | 0.7 | 0.311 | 0.778 | 0.667 | 1.056 | 1.567 | 2.378 |
| 36 | 1.278 | 1.489 | 2.311 | 2.822 | 3.333 | 3.056 | 2.589 |
| 4 | 2.39 | 2.68 | 2.67 | 2.02 | 1.91 | 1.56 | 1.41 |
| 14 | 2.46 | 2.56 | 2.96 | 2.44 | 2.18 | 2.29 | 2.37 |
| 15 | 2.72 | 3.01 | 3.02 | 2.10 | 2.18 | 1.62 | 1.62 |
| 16 | 2.96 | 3.17 | 3.12 | 2.90 | 2.68 | 2.61 | 2.61 |
| 17 | 2.49 | 2.70 | 2.99 | 2.34 | 2.34 | 1.53 | 1.63 |
| 18 | 2.56 | 2.81 | 2.70 | 2.66 | 2.66 | 2.62 | 2.02 |
| 19 | 2.57 | 2.78 | 2.78 | 2.78 | 2.56 | 2.09 | 2.01 |
| 20 | 2.678 | 2.667 | 2.744 | 3.211 | 2.967 | 2.5 | 2.5 |
| 21 | 3.067 | 3.278 | 3.167 | 2.233 | 2.089 | 1.978 | 1.833 |
| 22 | 3.5 | 3.711 | 3.711 | 3.133 | 3.022 | 3.022 | 2.8 |
| 23 | 3.589 | 3.844 | 4.067 | 3.522 | 3.256 | 3.211 | 3.022 |
| 34 | 2.167 | 2.378 | 2.378 | 2.156 | 2.011 | 2.389 | 2.522 |
| 37 | 3.122 | 3.333 | 3.333 | 3.333 | 3.333 | 3.056 | 2.589 |
| 31 | 2.244 | 2.233 | 2.456 | 2.456 | 2.756 | 2.756 | 2.233 |
| 24 | 3.389 | 3.678 | 3.678 | 3.033 | 2.922 | 2.567 | 2.422 |
| 28 | 3.267 | 3.478 | 3.478 | 2.922 | 3.067 | 3.067 | 2.778 |
| 25 | 2.544 | 2.756 | 2.256 | 2.256 | 2.322 | 1.811 | 1.522 |
| 38 | 2.39 | 2.68 | 2.67 | 2.02 | 1.91 | 1.56 | 1.41 |
| 29 | 2.39 | 2.68 | 2.67 | 2.02 | 1.91 | 1.56 | 1.41 |
| 32 | 2.39 | 2.68 | 2.67 | 2.02 | 1.91 | 1.56 | 1.41 |
| 35 | 2.46 | 2.56 | 2.96 | 2.44 | 2.18 | 2.29 | 2.37 |
| 26 | 2.244 | 2.233 | 2.456 | 2.456 | 2.756 | 2.756 | 2.233 |
| 39 | 3.411 | 3.622 | 3.622 | 3.622 | 3.578 | 3.578 | 3.578 |

TABLE 3
Average hydrophobicity and polarity of Cpds. 1-39 for amino
acids 1-18 of the N-terminal end and last nine amino acids
of the C-terminal end of the amino acid sequence of the signal
peptide and average polarity of Cpds. 1-39 for amino acids
1-9 of the N-terminal end of the signal peptide (continued)

| | | | | | Polarity |
|---|---|---|---|---|---|
| | | | | | Score |
| | amino | amino | amino | last nine | amino |
| | acids | acids | acids | amino | acids |
| Cpd. No. | 8-16 | 9-17 | 10-18 | acids | 1-9 |
| 1 | −0.086 | 0.5 | 1 | 1 | 7.79 |
| 2 | 2.511 | 2.8 | 2.9 | 2.3 | 6.352 |
| 3 | 1.822 | 1.767 | 1.256 | 1.256 | 6.344 |
| 5 | 3.211 | 3.022 | 3.022 | 1.144 | 6.107 |
| 6 | 1.822 | 1.867 | 1.2 | 1.144 | 0.824 |
| 7 | 2.1 | 1.878 | 1.211 | 0.7 | 12.178 |
| 8 | −1.578 | −1.256 | −1.267 | 1.356 | 23.538 |
| 9 | 2.844 | 2.322 | 2.589 | 0.2 | 11.466 |
| 10 | 2.756 | 2.233 | 2.367 | 0.133 | 6.548 |
| 11 | 2.022 | 2.244 | 2.256 | 1.711 | 12.024 |
| 12 | 0.378 | 0.889 | 1.489 | −0.311 | 1.272 |
| 13 | 2.733 | 1.922 | 1.622 | 1.622 | 0.949 |
| 30 | 2.756 | 2.233 | 2.367 | 0.133 | 6.548 |
| 27 | 2.022 | 2.244 | 2.256 | 1.711 | 12.024 |
| 33 | 2.156 | 2.011 | 2.389 | 0.622 | 1.14 |
| 36 | 2.633 | 1.856 | 1.778 | 1.778 | 6.621 |
| 4 | 1.70 | 1.06 | 0.79 | 0.79 | 0.783 |
| 14 | 2.37 | 1.86 | 1.63 | 1.11 | 0.641 |
| 15 | 1.62 | 1.08 | 0.61 | 0.61 | 0.664 |
| 16 | 2.10 | 1.50 | 1.28 | −0.04 | 0.43 |
| 17 | 1.67 | 0.78 | 0.42 | 0.42 | 0.659 |
| 18 | 1.46 | 1.40 | 1.43 | −0.67 | 0.454 |
| 19 | 2.01 | 1.34 | 1.12 | 1.12 | 0.731 |
| 20 | 1.989 | 1.356 | 1.356 | −0.378 | 0.231 |
| 21 | 1.622 | 1.511 | 1.511 | 0.656 | 0.436 |
| 22 | 2.9 | 2.678 | 2.122 | 1.833 | 0.419 |
| 23 | 3.022 | 2.467 | 1.656 | 1.144 | 0.26 |
| 34 | 1.711 | 1.711 | 1.867 | 0.622 | 0.646 |
| 37 | 2.633 | 1.856 | 1.778 | 1.778 | 0.56 |
| 31 | 2.367 | 1.767 | 0.956 | 0.133 | 0.64 |
| 24 | 2.211 | 1.567 | 1.3 | 1.3 | 0.443 |
| 28 | 2.778 | 2.178 | 2.178 | 1.711 | 0.41 |
| 25 | 1.3 | 0.944 | 0.722 | 0.722 | 0.599 |
| 38 | 1.70 | 1.06 | 0.79 | 0.79 | 0.783 |
| 29 | 1.70 | 1.06 | 0.79 | 0.79 | 0.783 |
| 32 | 1.70 | 1.06 | 0.79 | 0.79 | 0.783 |
| 35 | 2.37 | 1.86 | 1.63 | 1.11 | 0.641 |
| 26 | 2.367 | 1.767 | 0.956 | 0.133 | 0.64 |
| 39 | 3.111 | 2.889 | 2.6 | 1.556 | 0.26 |

TABLE 4
Average hydrophobicity and polarity of Cpds. 1-39 for amino acids 1-13 of
the N-terminal end and last nine amino acids of the C-terminal end of the
amino acid sequence of the signal peptide and average polarity of Cpds.
1-39 for amino acids 1-7 of the N-terminal end of the signal peptide

| | Hydrophobicity Score | | | | | | | | Polarity |
|---|---|---|---|---|---|---|---|---|---|
| | | | | | | | | last | Score |
| | amino | amino | amino | amino | amino | amino | amino | nine | amino |
| | acids | acids | acids | acids | acids | acids | acids | amino | acids |
| Cpd. No. | 1-7 | 2-8 | 3-9 | 4-10 | 5-11 | 6-12 | 7-13 | acids | 1-7 |
| 1 | 0.614 | 0.114 | 0.071 | 0.129 | 0.029 | 0.543 | 0.1 | 1 | 7.79 |
| 2 | 0.286 | −0.1 | 0.229 | 0.129 | 0.957 | 1.229 | 1.829 | 2.157 | 7.724 |
| 3 | 1.129 | 0.743 | 1.843 | 2.514 | 2.314 | 2.957 | 2.957 | 0.671 | 7.9 |
| 5 | 1.271 | 1.6 | 2.643 | 2.986 | 4.086 | 4.086 | 4.029 | 1.029 | 7.814 |
| 6 | 1.614 | 1.886 | 2.343 | 3.014 | 3.729 | 3.629 | 2.886 | 0.029 | 0.991 |
| 7 | −0.586 | −0.314 | 0.586 | 1.257 | 1.957 | 2.343 | 3.243 | 0.043 | 15.589 |
| 8 | −1.643 | −2.557 | −2.029 | −2.029 | −1.443 | −1.986 | −1.171 | 0.943 | 22.596 |
| 9 | −0.243 | 0.029 | 0.029 | 0.029 | 0.929 | 2.029 | 2.629 | −1.057 | 14.723 |
| 10 | 1.114 | 1.386 | 0.9 | 0.9 | 1.571 | 1.557 | 2.743 | −0.243 | 8.174 |
| 11 | −0.014 | −0.029 | −0.1 | −0.157 | 0.171 | 1.214 | 1.4 | 1.886 | 15.16 |
| 12 | −0.1 | −0.486 | −0.971 | −0.643 | −0.971 | −0.686 | −0.314 | 0.171 | 1.171 |
| 13 | 1.086 | 1.357 | 2.114 | 1.743 | 1.457 | 1.457 | 2.186 | 1.929 | 1.183 |
| 30 | 1.114 | 1.386 | 0.9 | 0.9 | 1.571 | 1.557 | 2.743 | −0.243 | 8.174 |
| 27 | −0.014 | −0.029 | −0.1 | −0.157 | 0.171 | 1.214 | 1.4 | 1.886 | 15.16 |
| 33 | 0.586 | 0.857 | 0.686 | −0.086 | 0.557 | 1.071 | 1.971 | 0.186 | 1.221 |
| 36 | 0.929 | 1.014 | 1.829 | 2.486 | 3.2 | 3.486 | 3.486 | 1.143 | 8.09 |
| 4 | 2.2 | 2.2 | 2.9 | 2.9 | 2.243 | 1.657 | 1.514 | 0.486 | 0.784 |
| 14 | 2.071 | 2.343 | 3 | 2.857 | 3.371 | 2.857 | 1.857 | 2 | 0.787 |
| 15 | 2.314 | 2.686 | 3.357 | 3.457 | 2.8 | 1.557 | 2.314 | 0.257 | 0.817 |
| 16 | 2.714 | 2.986 | 2.929 | 2.929 | 2.929 | 3.286 | 3.286 | −0.571 | 0.516 |
| 17 | 2.157 | 2.529 | 3.043 | 2.943 | 2.657 | 2.071 | 2.8 | −0.257 | 0.779 |
| 18 | 3.443 | 3.114 | 2.471 | 2.471 | 2.329 | 2.471 | 2.471 | −0.3 | 0.347 |
| 19 | 2.4 | 2.486 | 2.486 | 2.771 | 2.771 | 2.486 | 2.386 | 0.357 | 0.71 |
| 20 | 2.643 | 2.629 | 2.629 | 2.943 | 3.329 | 3.329 | 2.729 | −0.971 | 0.279 |
| 21 | 2.857 | 3.129 | 3.129 | 3.029 | 2.886 | 1.786 | 1.6 | 1 | 0.523 |
| 22 | 3.357 | 3.629 | 3.686 | 3.586 | 3.586 | 2.943 | 2.8 | 2.057 | 0.501 |
| 23 | 3.471 | 3.8 | 4.086 | 4.086 | 4.029 | 3.329 | 2.986 | 1.029 | 0.297 |
| 34 | 1.986 | 2.114 | 1.971 | 1.971 | 1.971 | 2.457 | 3.043 | 0.186 | 0.73 |
| 37 | 3.3 | 3.386 | 3.143 | 3.143 | 3.2 | 3.486 | 3.486 | 1.143 | 0.297 |
| 31 | 1.8 | 2.071 | 2.357 | 2.071 | 2.743 | 2.743 | 2.457 | −0.243 | 0.786 |
| 24 | 3.486 | 3.486 | 3.543 | 3.543 | 3.543 | 2.957 | 2.814 | 0.486 | 0.347 |
| 28 | 3.114 | 3.386 | 3.386 | 3.329 | 3.514 | 2.857 | 3.143 | 1.886 | 0.49 |
| 25 | 2.371 | 2.457 | 2.457 | 2.6 | 2.686 | 2.543 | 2.186 | 0.486 | 0.54 |
| 38 | 2.2 | 2.2 | 2.9 | 2.9 | 2.243 | 1.657 | 1.514 | 0.486 | 0.784 |
| 29 | 2.2 | 2.2 | 2.9 | 2.9 | 2.243 | 1.657 | 1.514 | 0.486 | 0.784 |
| 32 | 2.2 | 2.2 | 2.9 | 2.9 | 2.243 | 1.657 | 1.514 | 0.486 | 0.784 |
| 35 | 2.071 | 2.343 | 3 | 2.857 | 3.371 | 2.857 | 1.857 | 2 | 0.787 |
| 26 | 1.8 | 2.071 | 2.357 | 2.071 | 2.743 | 2.743 | 2.457 | −0.243 | 0.786 |
| 39 | 3.586 | 3.857 | 3.571 | 3.571 | 3.514 | 3.514 | 3.514 | 1.686 | 0.316 |

TABLE 5
Average hydrophobicity and polarity of Cpds. 1-39 for amino acids 1-9 of
the N-terminal end and last nine amino acids of the C-terminal end of the
amino acid sequence of the signal peptide and average polarity of Cpds.
1-39 for amino acids 1-5 of the N-terminal end of the signal peptide

| | Hydrophobicity Score | | | | | | Polarity |
|---|---|---|---|---|---|---|---|
| | | | | | | last | Score |
| | amino | amino | amino | amino | amino | nine | amino |
| | acids | acids | acids | acids | acids | amino | acids |
| Cpd. No. | 1-5 | 2-6 | 3-7 | 4-8 | 5-9 | acids | 1-5 |
| 1 | 0.26 | −0.28 | 0.56 | 1.02 | −0.02 | 0.34 | 10.546 |
| 2 | 1.26 | 0.8 | 0.1 | −0.96 | −0.26 | 2.1 | 0.914 |
| 3 | 0.12 | 0.3 | 1.98 | 2 | 1.92 | −0.38 | 10.964 |
| 5 | 0.1 | 0.56 | 2.1 | 2.58 | 4.12 | 0.32 | 10.888 |
| 6 | 0.46 | 0.98 | 1.96 | 2.9 | 3.62 | −0.18 | 1.336 |
| 7 | −0.82 | −2.1 | −0.5 | 0.44 | 1.14 | −0.66 | 11.398 |
| 8 | −1.88 | −3.04 | −1.78 | −2.36 | −1.62 | −0.08 | 21.734 |
| 9 | −0.18 | −0.64 | −1.08 | −1.08 | 0.18 | −1.08 | 20.612 |
| 10 | 1.7 | 0.42 | 0.82 | 0.82 | 0.68 | 0.06 | 1.018 |
| 11 | −0.2 | −0.08 | −0.32 | −0.8 | −0.34 | 1.96 | 20.612 |
| 12 | 0.7 | 0.18 | −0.88 | −0.96 | −0.96 | −0.88 | 0.602 |
| 13 | 1.92 | 1.28 | 1.3 | 1.68 | 1.82 | 1.38 | 1.002 |
| 30 | 1.7 | 0.42 | 0.82 | 0.82 | 0.68 | 0.06 | 1.018 |
| 27 | −0.2 | −0.08 | −0.32 | −0.8 | −0.34 | 1.96 | 20.612 |
| 33 | 0.76 | −0.32 | 0.52 | 0.52 | 0.34 | −0.02 | 0.978 |
| 36 | −0.22 | 0.16 | 1.56 | 2.22 | 2.88 | 0.08 | 11.274 |
| 4 | 2.46 | 2.84 | 2.84 | 2.32 | 2.4 | 0.38 | 0.74 |
| 14 | 1.38 | 1.76 | 2.68 | 2.68 | 3.6 | 1.14 | 1.05 |
| 15 | 2.56 | 2.02 | 3.04 | 3.18 | 3.18 | 0.36 | 0.784 |
| 16 | 2.6 | 2.58 | 2.58 | 2.58 | 2.58 | −0.9 | 0.696 |
| 17 | 2.44 | 1.8 | 2.8 | 2.8 | 2.6 | −0.86 | 0.742 |
| 18 | 3.3 | 3.68 | 3.68 | 2.76 | 1.86 | −0.54 | 0.434 |
| 19 | 3.02 | 3.14 | 2.22 | 2.36 | 2.36 | −0.22 | 0.364 |
| 20 | 2.18 | 2.56 | 2.56 | 3 | 3.4 | −0.42 | 0.338 |
| 21 | 3.56 | 3.94 | 2.86 | 2.72 | 2.72 | 0.34 | 0.39 |
| 22 | 3.56 | 3.94 | 3.56 | 3.42 | 3.5 | 1.76 | 0.39 |
| 23 | 3.26 | 3.72 | 4.12 | 4.12 | 4.04 | 0.32 | 0.364 |
| 34 | 2.34 | 1.64 | 1.64 | 1.44 | 1.24 | −0.02 | 0.68 |
| 37 | 3.1 | 3.48 | 3.4 | 3.14 | 2.88 | 0.08 | 0.364 |
| 31 | 2.08 | 2.46 | 1.78 | 1.78 | 2.72 | 0.06 | 0.758 |
| 24 | 3.36 | 3.74 | 3.74 | 3.22 | 3.3 | 0.38 | 0.434 |
| 28 | 3.24 | 3.22 | 3.22 | 3.14 | 3.4 | 1.96 | 0.66 |
| 25 | 2.2 | 2.18 | 2.18 | 2.12 | 3.14 | 0.26 | 0.73 |
| 38 | 2.46 | 2.84 | 2.84 | 2.32 | 2.4 | 0.38 | 0.74 |
| 29 | 2.46 | 2.84 | 2.84 | 2.32 | 2.4 | 0.38 | 0.74 |
| 32 | 2.46 | 2.84 | 2.84 | 2.32 | 2.4 | 0.38 | 0.74 |
| 35 | 1.38 | 1.76 | 2.68 | 2.68 | 3.6 | 1.14 | 1.05 |
| 26 | 2.08 | 2.46 | 1.78 | 1.78 | 2.72 | 0.06 | 0.758 |
| 39 | 3.5 | 3.88 | 3.88 | 3.88 | 3.4 | 0.6 | 0.39 |

In Vitro Transcription of mRNA

The IGF1_pVAX.A120 plasmid was linearized with XhoI and IGF1 mRNA (Cpd.1) was produced using the IVT system. Similarly, IGF1 mRNAs with altered pre-domains (signaling peptides, Cpd.2-Cpd.7 encoded in vectors pMA-T and pMA-RQ (FIG. 16) were produced at 50-200 μg range for in vitro transfection experiments using PCR based IVT. Likewise, for Cpd.1 (SEQ ID No. 40) and Cpd. 8-26 encoded in pMA-T and pMA-RQ vectors, IGF1 mRNAs with altered signaling peptides were produced at 50-200 μg range for in vitro transfection experiments using PCR based IVT. In addition to IGF1 mRNA, the mRNAs for erythropoietin (EPO, Cpd. 27-29), insulin (INS, Cpd. 30-32), interleukin 4 (IL4, Cpd. 33-35) and interleukin 10 (IL10, Cpd. 36-38) with endogenous or altered signaling peptide were produced at 50-200 μg for in vitro transfections experiments.

### In Vitro Transfection of HEK293T Cells for Testing of IGF1 Secretion

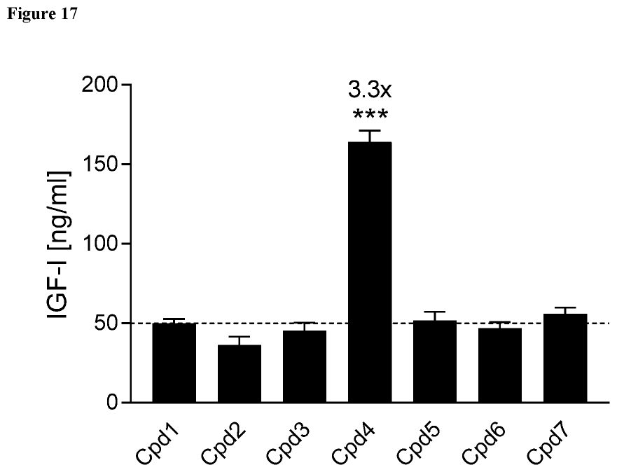

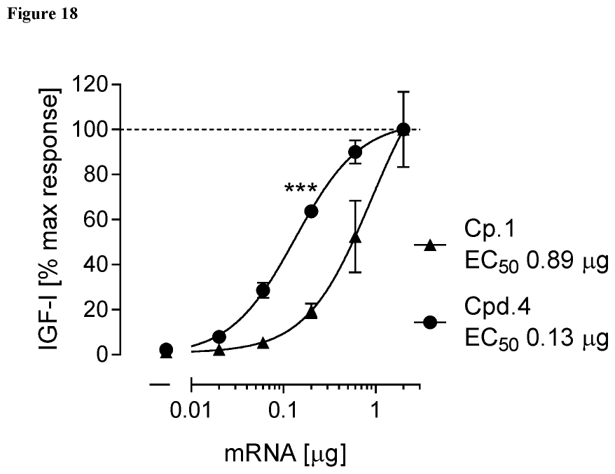

After 24 hours incubation of HEK293T cells with Cpd.1-Cpd.7 mRNAs, secreted IGF1 levels were assessed in the supernatants of the cell cultures (FIG. 17). Cpd.1 was capable of inducing IGF1 secretion up to 50 ng/ml. Cpd.4 induced IGF1 secretion significantly higher than Cpd.1 (3.3-fold, 0.001). To assess the concentration dependence of Cpd.1 and Cpd.4, different concentration of both Cpd.1 and Cpd.4 (0.02-2 μg/well) were tested for their induction of IGF1 secretion into the supernatant (FIG. 18). Cpd.1 revealed an EC₅₀ of 0.89 μg and for Cpd.4 and EC₅₀ of 0.13 μg, indicating that Cpd.4 was 6.8-fold more potent in inducing IGF1 secretion from HEK293T cells. Taken together, the data of FIGS. 17 and 18 demonstrate that Cpd.4 induced IGF1 secretion in HEK293T cells stronger and more potent than Cpd.1, suggesting that this signalling peptide facilitates cellular exit of produced IGF1 in this cell type.

### In Vitro Transfection of C2C12 Cells for Testing of IGF1 Secretion

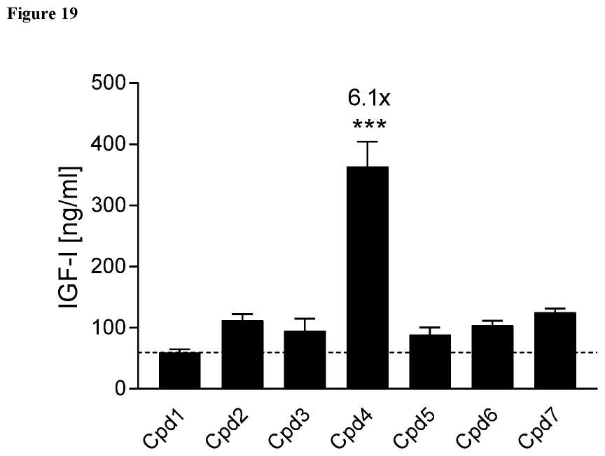

After 24 hours incubation of C2C12 cells with Cpd.1-Cpd.7 mRNAs, secreted IGF1 levels were assessed in the supernatants of the cell cultures (FIG. 19). Cpd.1 was capable of inducing IGF1 secretion up to 60 ng/ml. Cpd.4 induced IGF1 secretion significantly higher than Cpd.1 (6.1-fold, 0.001). The data demonstrate that Cpd.4 induced IGF1 secretion in C2C12 cells stronger than Cpd.1, suggesting that this signalling peptide facilitates cellular exit of produced IGF1 also in this cell type.

### In Vitro Transfection of HSkMC Cells for Testing of IGF1 Secretion

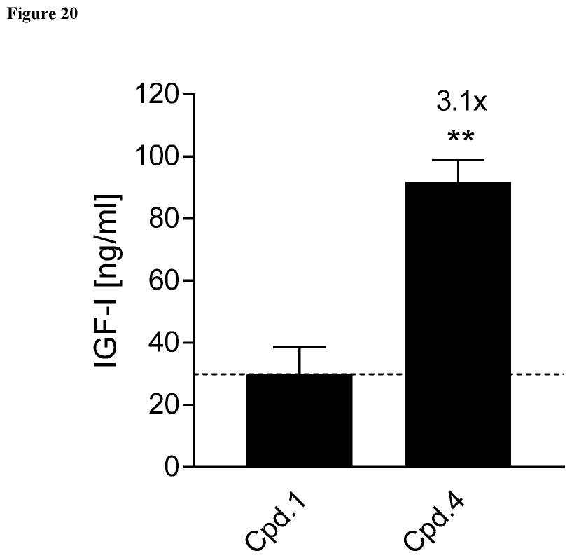

After 24 hours incubation of HSkMC cells with Cpd.1 or Cpd.4 mRNAs, secreted IGF1 levels were assessed in the supernatants of the cell cultures (FIG. 20). Cpd.1 was capable of inducing IGF1secretion up to 30 ng/ml. Cpd.4 induced IGF1 secretion significantly higher than Cpd.1 (3.1-fold, p<0.05). The data demonstrate that Cpd.4 induced IGF1 secretion in primary HSkMC cells stronger than Cpd.1, suggesting that this signalling peptide facilitates cellular exit of produced IGF1 also in this cell type.

In Vitro Transfection of Additional mRNAs in HEK293T Cells for Testing of IGF1 Secretion

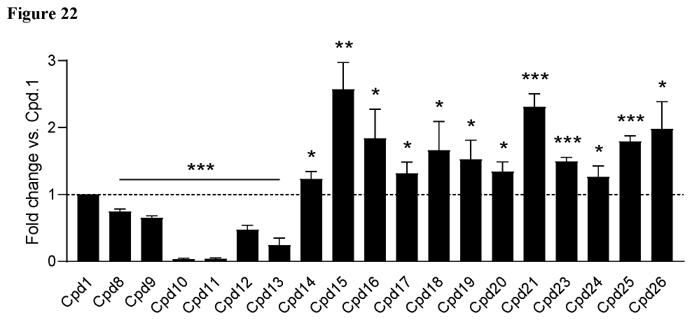

In another set of testings, Cpd.8-Cpd.26 were analyzed for their potential to modulated IGF1 secretion from HEK293T cells. After 24 hours incubation with Cpd.1 as control and Cpd.8-Cpd.26 as test mRNAs, secreted IGF1 levels were assessed in the supernatants of the cell cultures (FIG. 22). Cpd.1 response was normalized to 1 and data expressed as fold change of Cpd.1. Whereas Cpd.8, Cpd.9, Cpd.10, Cpd.11, Cpd.12 and Cpd13 showed a reduction of IGF1 secretion, Cpd.14, Cpd.15, Cpd.16, Cpd.17, Cpd.18, Cpd.19, Cpd.20, Cpd.21, Cpd.23, Cpd.24, Cpd.25 and Cpd.26 were capable of inducing IGF1 secretion significantly higher up to 2.6-fold compared to Cpd.1. Thereby, Cpd.15 and Cpd.21 showed similar induction as Cpd.4 (see FIG. 17). Taken together, the data demonstrate that Cpd.14, Cpd.15, Cpd.16, Cpd.17, Cpd.18, Cpd.19, Cpd.20, Cpd.21, Cpd.23, Cpd.24, Cpd.25 and Cpd.26 induced IGF1 secretion in HEK293T cells stronger and more potent than Cpd.1, suggesting that these signalling peptides facilitate cellular exit of produced IGF1 in this cell type.

### In Vitro Transfection of HepG2 Cells for Testing of IGF1 Secretion

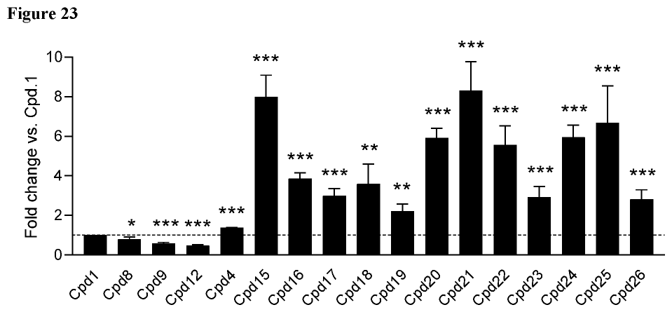

After 24 hours incubation of HepG2 cells with Cpd.1 as control and Cpd.4-Cpd.26 as test mRNAs, secreted IGF1 levels were assessed in the supernatants of the cell cultures (FIG. 23). Cpd.1 response was normalized to 1 and data expressed as fold change of Cpd.1. Whereas Cpd.8, Cpd.9, and Cpd.12 showed a reduction of IGF1 secretion, Cpd.4, Cpd.14, Cpd.15, Cpd.16, Cpd.17, Cpd.18, Cpd.19, Cpd.20, Cpd.21, Cpd.22, Cpd.23, Cpd.24, Cpd.25 and Cpd.26 were capable of inducing IGF1 secretion significantly higher up to 8.3-fold compared to Cpd.1. Taken together, the data demonstrate that Cpd.4, Cpd.14, Cpd.15, Cpd.16, Cpd.17, Cpd.18, Cpd.19, Cpd.20, Cpd.21, Cpd.22, Cpd.23, Cpd.24, Cpd.25 and Cpd.26 induced IGF1 secretion in HepG2 cells stronger than Cpd.1, suggesting that these signalling peptides facilitate cellular exit of produced IGF1 in this cell type.

### In Vitro Transfection of IMR32 Neuronal Cells for Testing of IGF1 Secretion

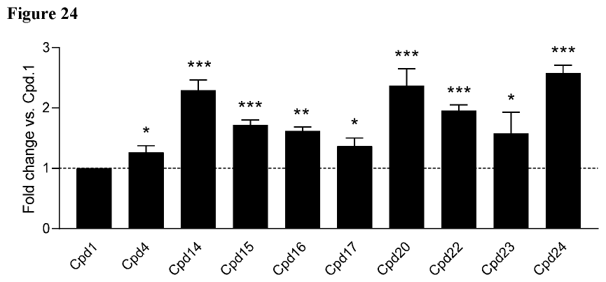

After 24 hours incubation of IMR324 neuronal cells with Cpd.1 as control and Cpd.4-Cpd.24 as test mRNAs, secreted IGF1 levels were assessed in the supernatants of the cell cultures (FIG. 24). Cpd.1 response was normalized to 1 and data expressed as fold change of Cpd.1. Cpd.4, Cpd.14, Cpd.15, Cpd.16, Cpd.17, Cpd.20, Cpd.22, Cpd.23 and Cpd.24 were capable of inducing IGF1 secretion significantly higher up to 2.6-fold compared to Cpd.1. Taken together, the data demonstrate that Cpd.4, Cpd.14, Cpd.15, Cpd.16, Cpd.17, Cpd.20, Cpd.22, Cpd.23 and Cpd.24 induced IGF1 secretion in IMR32 neuronal cells stronger than Cpd.1, suggesting that these signalling peptides facilitate cellular exit of produced IGF1 in this cell type.

### In Vitro Transfection of Human Chondrocytes for Testing of IGF1 Secretion

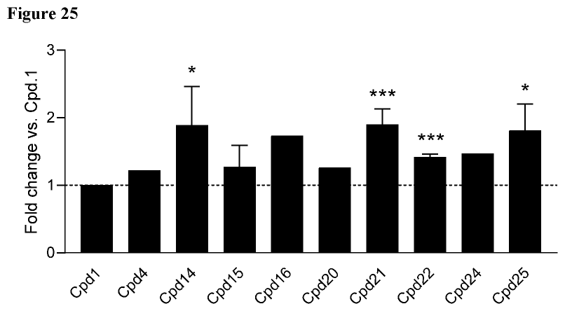

After 24 hours incubation of chondrocytes with Cpd.1 as control and Cpd.4-Cpd.25 as test mRNAs, secreted IGF1 levels were assessed in the supernatants of the cell cultures (FIG. 25). Cpd.1 response was normalized to 1 and data expressed as fold change of Cpd.1. Cpd.4, Cpd.14, Cpd.15, Cpd.16, Cpd.20, Cpd.21, Cpd.22, Cpd.24 and Cpd.25 were capable of inducing IGF1 secretion significantly higher up to 1.9-fold compared to Cpd.1. Taken together, the data demonstrate that Cpd.4, Cpd.14, Cpd.15, Cpd.16, Cpd.20, Cpd.21, Cpd.22, Cpd.24 and Cpd.25 induced IGF1 secretion in chondrocytes stronger than Cpd.1, suggesting that these signalling peptides facilitate cellular exit of produced IGF1 in this cell type.

### In Vitro Transfection of Rat Motoneurons for Testing of IGF1 Secretion

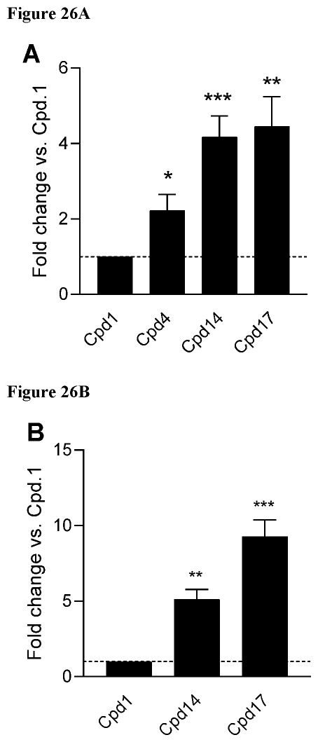

After 48 hours incubation of rat motoneurons or rat transgenic SOD1^{G93A} (FIG. 26B) with Cpd.1 as control and Cpd.4, Cpd.14 and Cpd.17 for wild type (FIG. 26A) or Cpd.14 and Cpd.17 for transgenic SOD1^{G93A} (FIG. 26B) as test mRNAs, secreted IGF1 levels were assessed in the supernatants of the cell cultures (FIGS. 26 A and 26B). Cpd.1 response was normalized to 1 and data expressed as fold change of Cpd.1. Cpd.4, Cpd.14 and Cpd.17 were capable of inducing IGF1 secretion up to 4.3-fold in wild-type or 9.3-fold in transgenic SOD1G^{S93A} compared to Cpd.1. Taken together, the data demonstrate that Cpd.4, Cpd.14 and Cpd.17 induced IGF1 secretion in motoneurons stronger than Cpd.1, suggesting that these signalling peptides facilitate cellular exit of produced IGF1 in this cell type.

### In Vitro Transfection of HEK293T, HepG2 and A549 Cells for Testing of EPO Secretion

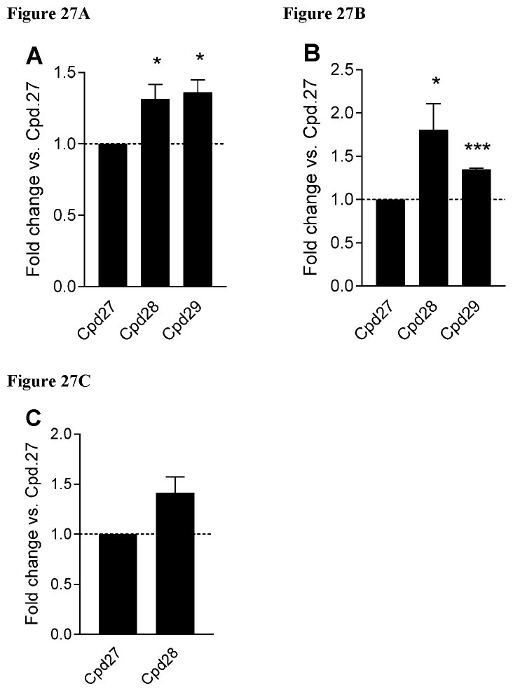

After 24 hours incubation of HEK293T, HepG2 or A549 cells with Cpd.27 as control and Cpd.28 and Cpd.29 as test mRNAs, secreted erythropoietin (EPO) levels were assessed in the supernatants of the cell cultures (FIGS. 27A-27C). Cpd.27 response was normalized to 1 and data expressed as fold change of Cpd.27. Cpd.28 was capable of inducing EPO secretion up to 1.8-fold compared to Cpd.27 in HEK293T cells (FIG. 27A), HepG2 cells (FIG. 27B) and A549 cells (FIG. 27C), Cpd.29 induced EPO secretion up to 1.4-fold compared to Cpd.27 in HEK293T cells (FIG. 27A) and HepG2 cells (FIG. 27B). Taken together, the data demonstrate that Cpd.28 and Cpd.29 induced EPO secretion stronger than Cpd.27, suggesting that these signalling peptides facilitate cellular exit of produced EPO in these cell types.

### In Vitro Transfection of HEK293T Cells for Testing of INS Secretion

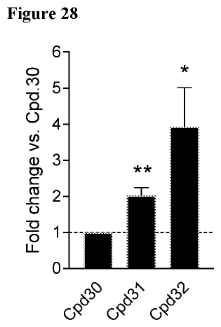

After 24 hours incubation of HEK293T cells with Cpd.30 as control and Cpd.31 and Cpd.32 as test mRNAs, secreted insulin (INS) levels were assessed in the supernatants of the cell cultures (FIG. 28). Cpd.30 response was normalized to 1 and data expressed as fold change of Cpd.30. Cpd.31 and Cpd.32 were capable of inducing INS secretion up to 3.9-fold compared to Cpd.30 in HEK293T cells. Taken together, the data demonstrate that Cpd.31 and Cpd.32 induced INS secretion stronger than Cpd.30, suggesting that these signalling peptides facilitate cellular exit of produced INS in this cell type.

### In Vitro Transfection of HEK293T, HepG2, THP-1 and A549 Cells for Testing of IL4 Secretion

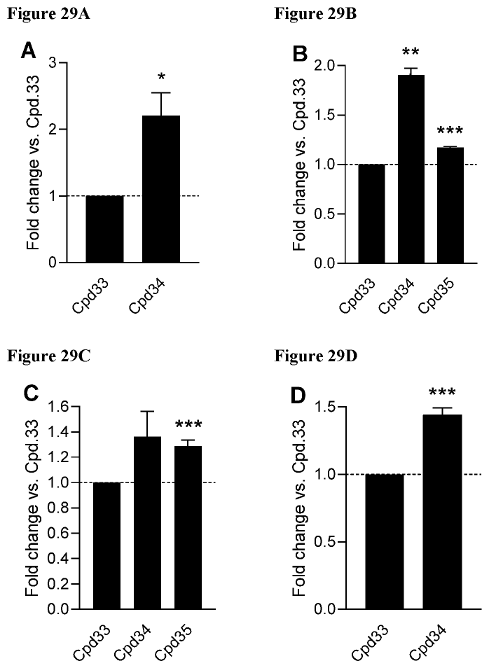

After 24 hours incubation of HEK293T, HepG2, THP-1 or A549 cells with Cpd.33 as control and Cpd.34 and Cpd.35 as test mRNAs, secreted interleukin 4 (IL4) levels were assessed in the supernatants of the cell cultures (FIGS. 29A-29D). Cpd.33 response was normalized to 1 and data expressed as fold change of Cpd.33. Cpd.34 was capable of inducing IL4 secretion up to 2.2-fold compared to Cpd.33 in HEK293T cells (FIG. 29A), HepG2 cells (FIG. 29B), THP-1 cells (FIG. 29C) and A549 cells (FIG. 29D), Cpd.35 induced IL4 secretion up to 1.3-fold compared to Cpd.33 in HepG2 cells (FIG. 29B) and THP-1 cells (FIG. 29C) respectively. Taken together, the data demonstrate that Cpd.34 and Cpd.35 induced IL4 secretion stronger than Cpd.33, suggesting that these signalling peptides facilitate cellular exit of produced IL4 in these cell types.

### In Vitro Transfection of HEK293T, HepG2 and THP-1 Cells for Testing of IL10 Secretion

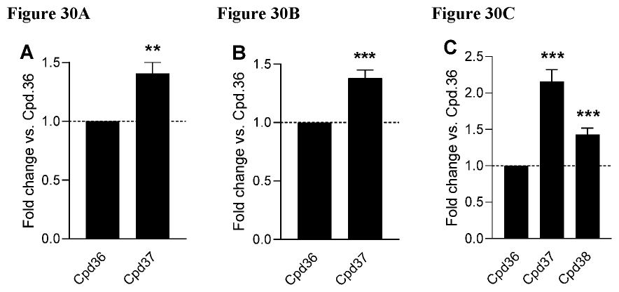

After 24 hours incubation of HEK293T, HepG2 or THP-1 cells with Cpd.36 as control and Cpd.37 and Cpd.38 as test mRNAs, secreted interleukin 10 (IL10) levels were assessed in the supernatants of the cell cultures (FIGS. 30A-30C). Cpd.36 response was normalized to 1 and data expressed as fold change of Cpd.36. Cpd.37 was capable of inducing IL10 secretion up to 2.2-fold compared to Cpd.36 in HEK293T cells (FIG. 30A), HepG2 cells (FIG. 30B) and THP-1 cells (FIG. 30C), Cpd.38 induced IL10 secretion 1.4-fold compared to Cpd.36 in THP-1 cells (FIG. 30C). Taken together, the data demonstrate that Cpd.37 and Cpd.38 induced IL10 secretion stronger than Cpd.36, suggesting that these signalling peptides facilitate cellular exit of produced IL10 in these cell types.

### In Vitro Transfection of Cpd.39 in HepG2 and Human Primary Chondrocytes Cells for Testing of IGF1 Secretion

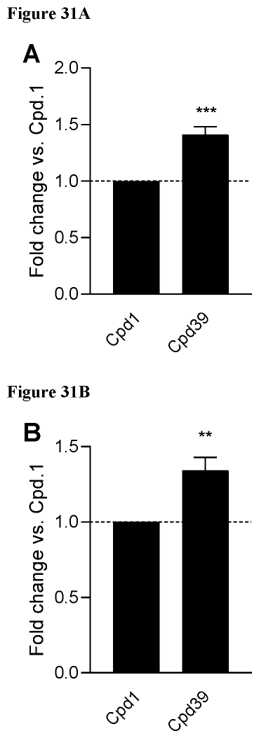

After 24 hours incubation of HepG2 or chondrocytes with Cpd.1 as control and Cpd.39 as test mRNAs, secreted IGF1 levels were assessed in the supernatants of the cell cultures (FIGS. 31A-31B). Cpd.1 response was normalized to 1 and data expressed as fold change of Cpd.1. Cpd.39, was capable of inducing IGF1 secretion significantly higher up to 1.4-fold compared to Cpd.1 in HepG2 (FIG. 31A) and human primary chondrocytes (FIG. 31B). Taken together, the data demonstrate that Cpd.39 induced IGF1 secretion stronger than Cpd.1, suggesting that this signaling peptide facilitate cellular exit of produced IGF1 in these cell type.

### Example 2

To test the efficacy of locally applied IGF-I mRNA in a mouse model of skeletal muscle injury, 8-10 weeks old male C57BL6/J mice were subjected to notexin-induced myotoxic injury in the tibialis anterior (TA) on day 0. On day 1 after injury, vehicle or 1 μg mRNA (Cpd.4) were applied via intramuscular injection into the injured muscle and repeated on day 4 after injury. Muscle function in the TA was measured at Day 1, 4, 7, 10, 14, 21 and 28 post-injury. A subset of contralateral TA muscles were also assessed throughout the study to assess the healthy control levels of TA muscle function.

### Methods and Material

Cloning of IGF-1 and In Vitro Transcription of IGF-1 mRNA

Cloning of IGF-1 and in vitro transcription of IGF-1 mRNA was carried out as described in example 1. Codon optimized DNA of Cpd. 4 (FIG. 4A) was used to be cloned in pMA-T vector to provide for the construct as shown in FIG. 11. This construct was used to produce in vitro transcribed mRNA used for mRNA treatment.

### Notexin Injury

Notexin (Latoxan, Valence, France) was prepared at a concentration of 0.4 μg in 40 μl saline for each mouse. Anesthesia in the mouse was induced in a chamber (˜4-5% isoflurane, to effect) and maintained via nose cone (˜2-3% isoflurane, to effect). The mouse was then maintained on a warmed (37° C.) surgical table. The skin over the mid belly of the TA muscle was prepared with depilatory cream (Nair Hair Remover for 45 seconds, followed by 3 times rinsing with water) and further prepped with three alternating scrubs of betadyne and 70% alcohol to prevent seeding skin bacteria into the soft tissue. Using a tuberculin syringe, 0.04 ml of prepared Notexin was injected intramuscularly into the belly of the right TA. The animals suffered no observable pain during the procedure and appropriate anti-pain treatment was administered subsequently (buprenorphine 0.05-0.1 mg/kg every 12h for 48h after injection). The first dose of analgesic was given at the time of anesthetic induction to ensure analgesic presence upon recovery. After the first 48h, the animal was examined at least twice weekly to assure proper healing and return of normal gait.

mRNA Treatment

Mice were randomly assigned to mRNA or vehicle treatment. Mice were anesthetized using isoflurane as described above, and the injured TA muscle was administered one intramuscular injection of mRNA or vehicle treatment in the middle of the muscle.

### Assessment of In Vivo TA Function

Muscle performance was measured in vivo with a 305C muscle lever system (Aurora Scientific Inc., Aurora, Canada) at Day 1, 4, 7, 10, 14, 21 and 28 post-injury. Anesthesia in the mouse was accomplished as described above, and the mouse was placed on a thermostatically controlled table. The knee was isolated using a pin pressed against the tibial head and the foot firmly fixed to a footplate on the motor shaft. For the dorsiflexor muscle group, contractions were elicited by percutaneous electrical stimulation of the peroneal nerve. Optimal isometric twitch torque was determined by increasing the current with a minimum of 30s between each contraction to avoid fatigue. A series of stimulations were then performed at increasing frequency of stimulation (0.2 ms pulse, 500 ms train duration): 1, 10, 20, 40, 60, 80, 100, 150 Hz, followed by a final stimulation at 1 Hz. Maximal peak isometric force was plotted.

### Data Analysis

Data were grouped and means and standard errors were calculated. Statistical analysis was performed using GraphPad Prism 8 (San Diego, USA). Comparisons were made using a student's t-test.

### Results

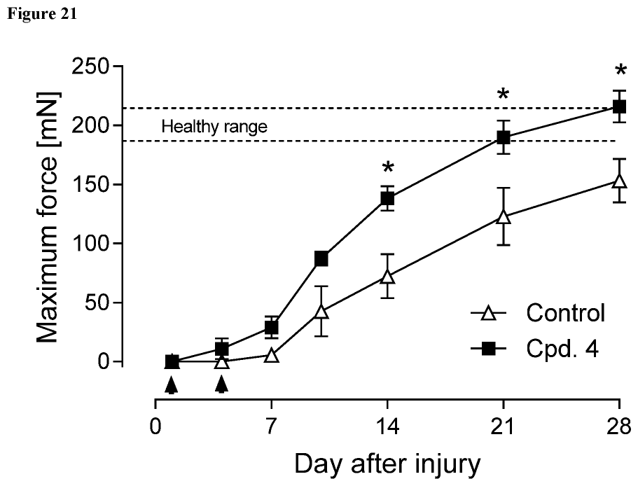

Example 2 shows that in a mouse model of TA muscle injury induced by a toxin (notexin), IGF-I mRNA intramuscular treatment early after muscle injury on days 1 and 4 resulted in an accelerated and full recovery of muscle function (FIG. 21). Animals treated with 1 μg Cpd. 4 reached functional levels in the healthy range by 16 days. In contrast, control animals treated with vehicle and lower dose-treated mice did not even achieve full functional recovery by day 28. The data therefore indicate that IGF-I mRNA treatment can accelerate the healing after muscle injury and can potentially prevent a chronic impairment by fully recovering muscle function. Thereby, surprisingly only two doses early after the injury are necessary.

1.-54. (canceled)

55. A composition comprising a recombinant RNA construct or a vector encoding the recombinant RNA construct, wherein the recombinant RNA construct comprises a first nucleic acid sequence encoding a signal peptide operably linked to a second nucleic acid sequence encoding a protein of interest,
wherein the second nucleic acid sequence encoding the protein of interest comprises a coding sequence of a mature protein of the protein of interest;
wherein amino acids 1-9 of the N-terminal end of the amino acid sequence of the signal peptide have an average hydrophobicity score of above 2, as calculated according to the Kyte-Doolittle scale; and
wherein the signal peptide is a signal peptide heterologous to the protein of interest, with the proviso that the protein of interest is not an oxidoreductase.

56. The composition of claim 55, wherein amino acids 1-9 of the N-terminal end of the amino acid sequence of the signal peptide have an average hydrophobicity score of between 2.0 and 4.0, as calculated according to the Kyte-Doolittle scale.

57. The composition claim 55, wherein amino acids 1-9 of the N-terminal end of the amino acid sequence of the signal peptide have an average polarity of 6.1 or below, as calculated according to Zimmerman Polarity index.

58. The composition of claim 55, wherein the protein of interest is secreted, and wherein the quantity of the secreted protein of interest is higher than the quantity of the secreted protein of interest using a signal peptide homologous to the protein of interest.

59. The composition of claim 55, wherein the signal peptide heterologous to the protein of interest is selected from the group consisting of the signal peptide of insulin growth factor 2 (IGF1), the signal peptide of insulin growth factor 2 (IGF2), the signal peptide of serum albumin (ALB), the signal peptide of brain-derived neurotrophic factor (BDNF), the signal peptide of C-X-C Motif Chemokine Ligand 12 (CXCL12), the signal peptide of latent-transforming growth factor beta-binding protein 2 (LTBP2), the signal peptide of Insulin-like growth factor-binding protein complex acid labile subunit (IGFALS), the signal peptide of insulin (INS), the signal peptide of erythropoietin (EPO), the signal peptide of granulocyte colony-stimulating factor (CSF3), the signal peptide of beta-nerve growth factor (NGF), the signal peptide of fibroblast growth factor 5 (FGF5), the signal peptide of complement factor H-related protein 2 (FHR2), the signal peptide of insulin-like growth factor binding protein 5 (IBP5), the signal peptide of neurotrophin-3 (NTF3), the signal peptide of prostate and testis expressed protein 2 (PATE2), and the signal peptide of extracellular superoxide dismutase (SOD3).

60. The composition of claim 55, wherein the signal peptide heterologous to the protein of interest is a modified signal peptide comprising an insertion, deletion, and/or substitution of at least one amino acid.

61. The composition of claim 60, wherein the protein of interest is secreted when expressed in a cell, and wherein the quantity of the secreted protein of interest is higher than the quantity of a secreted protein of interest comprising a signal peptide homologous to the protein of interest expressed in a cell.

62. The composition of claim 55, wherein the protein of interest comprises a cytokine, a growth factor, or a hormone.

63. The composition of claim 62, wherein the cytokine, the growth factor, or the hormone is selected from the group consisting of insulin growth factor 1 (IGF1), insulin (INS), erythropoietin (EPO), interleukin 4 (IL4), and interleukin 10 (IL10).

64. The composition of claim 55, wherein the recombinant RNA construct encodes a protein comprising an amino acid sequence with at least 80% sequence identity to an amino sequence encoded by a sequence selected from the group consisting of SEQ ID NOs: 4, 6, 8, 10, 44, 49, 54, 59, 64, 69, 90, 95, 100, 105, 110, 115, 158, 164, 172, and 180.

65. The composition of claim 55, wherein the composition comprises the recombinant RNA construct, wherein the recombinant RNA construct is a recombinant messenger RNA (mRNA) construct.

66. The composition of claim 55, wherein the composition comprises the recombinant RNA construct, wherein the recombinant RNA construct further comprises an anti-reverse CAP analog (ARCA) and/or an internal ribosome entry site (IRES) at the 5′ end.

67. The composition of claim 55, wherein the recombinant RNA construct further comprises a polyA tail at the 3′ end.

68. The composition of claim 67, wherein the polyA tail is 120 bp in length.

69. A pharmaceutical composition comprising the composition of claim 55 and pharmaceutically acceptable excipient, carrier, or diluent.

70. A method of treating a disease or a condition in a human subject in need thereof, comprising administering to the human subject a therapeutically effective amount of the pharmaceutical composition of claim 69.

71. The method of claim 70, wherein the disease or the condition comprises a skeletal muscle injury.

72. The method of claim 70, wherein the pharmaceutical composition is formulated as a liquid or wherein the administering comprises an intramuscular injection.

73. A method of synthesizing the composition of claim 55, wherein the method comprises synthesizing a recombinant RNA construct in vitro, wherein the recombinant RNA construct comprises a first nucleic acid sequence encoding a signal peptide operably linked to a second nucleic acid sequence encoding a protein of interest,
wherein the second nucleic acid sequence encoding the protein of interest comprises a coding sequence of a mature protein of the protein of interest;
wherein amino acids 1-9 of the N-terminal end of the amino acid sequence of the signal peptide have an average hydrophobicity score of above 2, as calculated according to the Kyte-Doolittle scale; and
wherein the signal peptide is a signal peptide heterologous to the protein of interest, with the proviso that the protein of interest is not an oxidoreductase.

74. A composition comprising a recombinant RNA construct comprising a first nucleic acid sequence encoding a signal peptide operably linked to a second nucleic acid sequence encoding a protein of interest,
wherein the second nucleic acid sequence encoding the protein of interest comprises a coding sequence of a mature protein of the protein of interest;
wherein the amino acids 1-9 of the N-terminal end of the amino acid sequence of the signal peptide have an average hydrophobicity score of above 2, as calculated according to the Kyte-Doolittle scale;
wherein the signal peptide is a signal peptide homologous to the protein of interest; and
wherein the signal peptide homologous to the protein of interest is a modified signal peptide comprising an insertion, deletion and/or substitution of at least one amino acid.
